(19) United States
(12) Patent Application Publication
(10) Pub. No.: US 2026/0207136 A1
(43) Pub. Date: Jul. 23, 2026

(54) EAR-WEARABLE HEALTH MONITORING SYSTEM

(71) Applicant: OXIWEAR, INC., Arlington, VA (US)

(72) Inventors: Shavini Evendri FERNANDO, Arlington, VA (US); George Donald BECKSTEIN, III, Lancaster, NY (US); Michael Maximo BONCALDO, Amherst, NY (US); Erik Rene CALDERON, Port Washington, NY (US)

(21) Appl. No.: 19/410,816

(22) Filed: Dec. 5, 2025

Related U.S. Application Data
(60) Provisional application No. 63/728,479, filed on Dec. 5, 2024.

(51) Int. Cl. A61B 5/00; A61B 5/0205; A61B 5/024; A61B 5/11; A61B 5/1455; G16H 40/67

(52) U.S. Cl. CPC A61B 5/6803; A61B 5/02055; A61B 5/4088; A61B 5/4806; A61B 5/7275; A61B 5/7455; A61B 5/746; A61B 5/747; G16H 40/67; A61B 5/02433; A61B 5/11; A61B 5/14552; A61B 2560/0209; A61B 2560/0242

(57) ABSTRACT

A system includes a wearable device and a charger case. The wearable device is configured to be positioned on an ear of a wearer, and includes first and second body portions and a connection member mechanically coupled to each of the first body portion and the second body portion. The first body portion and/or the second body portion includes a processor, a memory, and a sensor set including at least one of a pulse oximeter, a photoplethysmogram (PPG) sensor, an accelerometer, a temperature sensor, a proximity sensor, an ambient light sensor, or a gyroscopic sensor. The memory stores instructions to cause the processor to calculate at least one of a blood oxygen saturation level, a pulse rate, a blood pressure level, a blood flow rate, a cardiac output/rhythm, a blood sugar level, a blood carbon monoxide level, a blood nitrous oxide level, or an electrocardiogram of the wearer.

### CROSS-REFERENCE TO RELATED APPLICATION

This application claims priority to and the benefit of U.S. Provisional Patent Application No. 63/728,479, filed on Dec. 5, 2024 and titled “EAR-WEARABLE HEALTH MONITORING SYSTEM,” the entire disclosure of which is hereby incorporated by reference herein.

### FIELD

The present disclosure relates to physiological monitoring technology, and more specifically, to the monitoring of a health parameter(s) such as an oxygen level of a wearer of a device.

### BACKGROUND

Wearable medical devices and fitness trackers can be useful for monitoring a health parameter(s) of patients and other wearers. For example, a condition(s) such as pulmonary hypertension can be treated by providing a patient with supplemental oxygen therapy. The supplemental oxygen therapy can include delivering oxygen to the patient continuously, or during specific events such as exercise or sleep.

### SUMMARY

In some embodiments, an apparatus includes a wearable device configured to be positioned on an ear of a wearer. The wearable device includes a first body portion, a second body portion, and a connection member mechanically coupled to each of the first body portion and the second body portion. At least one of the first body portion or the second body portion has a processor, a memory operably coupled to the processor, and a sensor set including at least one of a pulse oximeter, a photoplethysmogram (PPG) sensor, an accelerometer, a temperature sensor, a proximity sensor, an ambient light sensor, or a gyroscopic sensor. The memory stores instructions to cause the processor to at least two of: (i) calculate a blood oxygen saturation level of the wearer; (ii) calculate a pulse rate of the wearer; (iii) calculate a blood pressure level of the wearer; (iv) calculate a blood flow rate of the wearer; (v) identify a cardiac output/rhythm of the wearer; (vi) calculate a blood sugar level of the wearer; (vii) calculate a blood carbon monoxide level of the wearer; (viii) calculate a blood nitrous oxide level of the wearer; (ix) generate an electrocardiogram associated with the wearer; (x) estimate a risk of one of dementia or Alzheimer's disease of the wearer by analyzing at least one of blood pressure variability (BPV), pulse wave velocity (PWV), and pulse transit time (PTT); or (xi) detect peripheral artery disease (PAD) of the wearer based on a pulse wave form detected via the wearable device.

In some embodiments, the wearable device includes a first light-emitting diode configured to emit light at a first wavelength and a second light-emitting diode configured to emit light at a second wavelength different from the first wavelength. At least one optical sensor may be configured to detect reflected portions of the first and second light from a surface of the wearer's ear. In some embodiments, the first wavelength is within a red light spectrum and the second wavelength is within an infrared spectrum, and the processor is configured to calculate the wearer's blood oxygen saturation level based on the Beer-Lambert law using the amount of light absorbed by the surface of the wearer's ear.

In some embodiments, the sensor set includes a photoplethysmography (PPG) sensor configured to determine pulse-wave signals based on measured time intervals between heartbeats. The PPG sensor may include one or more light-emitting diodes configured to emit light at a tissue site of the wearer's ear and a photodiode configured to measure the intensity of non-absorbed light reflected from the tissue site. In some embodiments, the one or more light-emitting diodes include at least one of a red LED, a green LED, or a yellow LED.

In some embodiments, the sensor set includes an accelerometer and a gyroscopic sensor configured to detect at least one of body position, head position, or falls of the wearer. The wearable device may also include an ambient light sensor configured to adjust the intensity of light emitted by the first and second light-emitting diodes based on detected ambient light conditions. In some embodiments, the sensor set includes a proximity sensor configured to detect the positioning of the wearable device relative to the wearer's ear.

In some embodiments, the wearable device includes a vibration motor operably coupled to the processor, and the memory stores instructions to cause the processor to operate the vibration motor in response to detecting an alarm condition. The wearable device may also include a wireless transceiver operably coupled to the processor and configured to communicate with a mobile software application. In some embodiments, the memory stores instructions to cause the processor to transmit measurement data to the mobile software application via the wireless transceiver using at least one of Bluetooth®, LTE, or Wi-Fi protocol.

In some embodiments, the memory stores instructions to cause the processor to generate an alert when the calculated blood oxygen saturation level is below a predefined threshold, the alert including at least one of an audible sound, a vibration, or a visual indication. The wearable device may further include an alert mechanism operably coupled to the processor, and the memory stores instructions to cause the processor to initiate an emergency plan in response to detecting a user interaction with the alert mechanism. In some embodiments, the emergency plan includes at least one of initiating a telephone call to emergency services, sending a short message service (SMS) message to a predefined contact, or transmitting an alert signal to a mobile software application.

In some embodiments, the memory stores instructions to cause the processor to estimate the risk of dementia or Alzheimer's disease by analyzing at least one of blood pressure variability, pulse wave velocity, or pulse transit time detected via the sensor set. The wearable device may include a temperature sensor configured to measure the wearer's skin temperature and adjust calculations of blood oxygen saturation based on the detected temperature. In some embodiments, the wearable device includes a battery voltage sensor configured to regulate power consumption by adjusting the operation of the sensor set based on the wearer's activity level.

In some embodiments, the memory stores instructions to cause the processor to generate a sleep report based on data collected from at least two sensors in the sensor set. The wearable device may further include at least one microphone and at least one speaker operably coupled to the processor, and the memory stores instructions to cause the processor to activate the microphone and the speaker in response to initiating an emergency plan. In some embodiments, the wearable device is configured to continuously monitor the wearer's blood oxygen saturation level and dynamically adjust a sampling rate of the sensor set based on detected changes in the wearer's physiological condition.

In some embodiments, a system includes a wearable device and a charger case. The wearable device is configured to be positioned on an ear of a wearer, and includes a first body portion, a second body portion, and a connection member mechanically coupled to each of the first body portion and the second body portion. At least one of the first body portion or the second body portion includes a processor, a memory operably coupled to the processor, and a sensor set including at least one of a pulse oximeter, a photoplethysmogram (PPG) sensor, an accelerometer, a temperature sensor, a proximity sensor, an ambient light sensor, or a gyroscopic sensor. The memory stores instructions to cause the processor to calculate at least one of a blood oxygen saturation level, a pulse rate, a blood pressure level, a blood flow rate, a cardiac output/rhythm, a blood sugar level, a blood carbon monoxide level, a blood nitrous oxide level, or an electrocardiogram of the wearer. The charger case is configured to contain and supply power to the wearable device, the charger case being at least one of waterproof or wirelessly chargeable.

In some embodiments, the charger case includes a pair of electrical contacts configured to exchange at least one of power or data with the wearable device when the wearable device is positioned within the charger case. In some embodiments, the charger case is configured to support wireless charging of the wearable device when the wearable device is contained within the charger case. The charger case may be waterproof and have an ingress protection rating of at least IP67.

In some embodiments, the charger case includes a battery configured to supply power to the wearable device and a battery monitoring circuit operably coupled to the battery. The charger case may further include a lid portion and a base portion coupled by a hinge, with the lid portion configured to enclose the wearable device when the charger case is in a closed configuration. In some embodiments, the charger case includes at least one LED indicator configured to display a charging status of the wearable device.

In some embodiments, the wearable device includes a first light-emitting diode configured to emit light at a first wavelength, a second light-emitting diode configured to emit light at a second wavelength different from the first wavelength, and at least one optical sensor configured to detect a reflected portion of the first light and a reflected portion of the second light from a surface of the wearer's ear. In some embodiments, the wearable device includes a first light-emitting diode configured to emit light at a first wavelength and a second light-emitting diode configured to emit light at a second wavelength different from the first wavelength, and the sensor set includes an ambient light sensor configured to adjust an intensity of light emitted by the first and second light-emitting diodes based on detected ambient light conditions.

In some embodiments, the charger case includes a universal serial bus (USB) port configured to enable wired charging of the charger case. The wearable device may further include an alert mechanism operably coupled to the processor, and the memory stores instructions to cause the processor to initiate an emergency plan in response to detecting a user interaction with the alert mechanism. In some embodiments, the wearable device includes at least one microphone and at least one speaker operably coupled to the processor, and the memory stores instructions to cause the processor to activate the microphone and the speaker in response to initiating an emergency plan.

In some embodiments, a system includes a wearable device, a charger case, and a plurality of flexible earpads. The wearable device is configured to be positioned on an ear of a wearer. The wearable device includes a first body portion, a second body portion, and a connection member mechanically coupled to each of the first body portion and the second body portion. At least one of the first body portion or the second body portion includes a processor, a memory operably coupled to the processor, a first light-emitting diode, a second light-emitting diode, and at least one optical sensor configured to detect, during operation of the wearable device, a reflected portion of first light emitted from the first light-emitting diode and a reflected portion of second light emitted from the second light-emitting diode, each of the reflected portion of the first light and the reflected portion of the second light being reflected from an associated surface of the ear of the wearer. The memory stores instructions to cause the processor to calculate at least one of at least one of a blood oxygen saturation level, a pulse rate, a blood pressure level, a blood flow rate, a cardiac output/rhythm, a blood sugar level, a blood carbon monoxide level, a blood nitrous oxide level, or an electrocardiogram based on at least one of the reflected portion of the first light or the reflected portion of the second light. The charger case is configured to contain the wearable device and to supply power to the wearable device, the charger case being at least one of waterproof or wireless. Each flexible earpad from the plurality of flexible earpads is configured to be mechanically coupled to one of the first body portion or the second body portion. In some such implementations, the wearable device is in wireless communication with a mobile compute device (e.g., a smartphone) running a software application during operation of the wearable device. The wearable device may further include at least one microphone and at least one speaker operably coupled to the processor, and the memory stores instructions to cause the processor to activate the microphone and the speaker in response to initiating an emergency plan.

In some embodiments, the charger case includes a pair of electrical contacts configured to exchange at least one of power or data with the wearable device when the wearable device is positioned within the charger case. The charger case may also be configured to support wireless charging of the wearable device when the wearable device is contained within the charger case. In some embodiments, the charger case is waterproof and has an ingress protection rating of at least IP67. The charger case may further include a battery configured to supply power to the wearable device and a battery monitoring circuit operably coupled to the battery.

In some embodiments, each flexible earpad from the plurality of flexible earpads is configured to be interchangeable with another flexible earpad having different dimensions to customize a fit of the wearable device to the wearer's ear. Each flexible earpad may be formed from a silicone material.

In some embodiments, the wearable device includes a first light-emitting diode configured to emit light at a first wavelength and a second light-emitting diode configured to emit light at a second wavelength different from the first wavelength, and the sensor set includes an ambient light sensor configured to adjust an intensity of light emitted by the first and second light-emitting diodes based on detected ambient light conditions. The wearable device may further include an alert mechanism operably coupled to the processor, and the memory stores instructions to cause the processor to initiate an emergency plan in response to detecting a user interaction with the alert mechanism. In some embodiments, the emergency plan includes at least one of initiating a telephone call to emergency services, sending a short message service (SMS) message to a predefined contact, or transmitting an alert signal to a mobile software application.

In some embodiments, the memory stores instructions to cause the processor to generate a sleep report based on data collected from at least two of a photoplethysmogram sensor, an accelerometer, or a gyroscopic sensor.

### BRIEF DESCRIPTION OF THE DRAWINGS

FIGS. 1A-1C are renderings of a wearable oxygen monitor, configured to be worn about a portion of an ear of a user, according to some embodiments.

FIGS. 1D-1E are exploded-view renderings of the wearable monitor of FIGS. 1A-1C, showing components thereof.

FIGS. 1F-1J are renderings of the wearable monitor of FIGS. 1A-1C and a charger case for the wearable monitor of FIGS. 1A-1C.

FIGS. 2A-2N are drawings of a wearable oxygen monitor, configured to be worn about a portion of an ear of a user, according to some embodiments.

FIGS. 2O-2V are drawings of a charger case in a closed configuration, the charger case configured to contain, and to supply power to, the wearable monitor of FIGS. 2A-2N, according to some embodiments.

FIGS. 2W-2AD are drawings of the charger case of FIGS. 2O-2V in an open configuration, the charger case containing the wearable monitor of FIGS. 2A-2N.

FIGS. 2AE-2AF are drawings of the charger case of FIGS. 2O-2V in an open configuration and not containing a wearable oxygen monitor.

FIG. 2AG is a rendering of a charger case with a panel removed to show an underlying shape of a recess/pocket configured to receive a wearable oxygen monitor, according to some embodiments.

FIG. 3 is a schematic drawing showing components of a wearable oxygen monitor, according to some embodiments.

FIG. 4 is a block diagram showing components of a wearable oxygen monitor, according to some embodiments.

FIG. 5 is a block diagram showing components of a charger case, according to some embodiments.

FIG. 6 is a drawing of a wearable monitor when worn about an upper portion of an ear of a user/wearer, according to an embodiment.

FIGS. 7A-7C are renderings of a wearable oxygen monitor, configured to be worn about a portion of an ear of a user, at various stages of assembly and showing components thereof, according to some embodiments.

FIGS. 8A-8U are renderings of a flexible electronics assembly for a wearable oxygen monitor, according to some embodiments.

FIGS. 9A-9B are renderings of an earpad with optical sensor circuitry positioned within a recess of the earpad, according to some embodiments.

FIG. 9C is a photographic image showing an underside view of an assembled earpad with optical sensor circuitry, according to an embodiment.

FIGS. 10A-10C are drawings of a wearable oxygen monitor, showing a grommet without an earpad, with a thin earpad, and with a thick earpad, respectively, according to some embodiments.

FIG. 11 is a flow diagram showing a method of manufacturing a wearable oxygen monitor, according to some embodiments.

FIGS. 12-60 are wireframes of user interface screens of a mobile app that interacts with a wearable oxygen monitor, according to some embodiments.

FIGS. 61A-61N are renderings of a flexible electronics assembly for a wearable monitor and components therein, according to some embodiments.

FIG. 62A illustrates various vital signs, biometric parameters, and physiological or physical conditions (e.g., body position) that can be measured, detected, or estimated by one or more wearable health monitors, according to some embodiments.

FIG. 62B illustrates the hardware components and sensors that may be integrated with or incorporated into one or more wearable health monitors, according to some embodiments.

FIG. 62C illustrates how user information can be input into one or more wearable health monitors, according to some embodiments.

FIG. 62D illustrates the analytics capabilities that can be generated by or used with one or more wearable health monitors, according to some embodiments.

FIG. 62E illustrates a variety of use cases across different sectors (e.g., healthcare, sports) where one or more wearable health monitors can be applied, according to some embodiments.

FIG. 62F illustrates features of a mobile software application that can be integrated with or used in conjunction with one or more wearable health monitors, according to some embodiments.

### DETAILED DESCRIPTION

Some health conditions, such as pulmonary hypertension (PH), pulmonary arterial hypertension (PAH), idiopathic PAH (IPAH), and acute mountain sickness (AMS) are managed through the administration of oxygen and, relatedly, the monitoring of oxygen levels. Known devices for monitoring oxygen levels (such as pulse oximeters), however, are typically worn on the finger of a patient for discrete periods of time for measurement, and then taken off, for example because they are not designed or comfortable for continuous wear. Moreover, when a patient sleeps, he or she is not typically wearing a pulse oximeter, and is unable to view a digital readout of the pulse oximeter. As such, known pulse oximeters may not be effective for a notifying the patient of a critical drop in his or her oxygen level, for example while he/she sleeps, potentially leading to an exacerbation of the condition or even death. Moreover, known pulse oximeters do not include automated emergency detection and response capabilities. In other words, a user make take a voluntary action to measure his/her oxygen level, and upon determining that the level is too low, take another voluntary action to address it (e.g., call a doctor or emergency services using another device such as a telephone), if he/she is capable of doing so, potentially wasting valuable time.

Embodiments of the present disclosure include a wearable monitor that can be continuously worn and perform continuous oxygen monitoring and to alert a wearer/user when a detected oxygen level, detected during the oxygen monitoring, is lower than a predetermined or predefined threshold value. In some embodiments, the wearable monitor includes a first body portion, a second body portion, and a connection member or a connection portion that is disposed or positioned between the first body portion and the second body portion. The first body portion includes a processor, a memory operably coupled to the processor, at least one light-emitting diode (LED), and at least one optical sensor configured to detect, during operation, a reflected portion of light emitted from the at least one light-emitting diode. The second body portion includes a grommet, and the connection member is mechanically coupled to each of the first body portion and the second body portion. The memory stores instructions to cause the processor to cause light to be emitted from the at least one light-emitting diode when the apparatus is positioned on an ear of a wearer and during operation of the apparatus, and to calculate a blood oxygen saturation level of the wearer based on the reflected portion of light. Additional details about certain aspects of the wearable monitors described herein can be found, by way of example, in U.S. Pat. No. 10,987,067, titled “Wearable Earpiece Oxygen Monitor,” issued Apr. 27, 2021, the entire contents of which are incorporated herein by reference for all purposes.

In some implementations, the connection member includes or is made of nylon, and thus is only slightly compliant (but primarily rigid), and an adjustability of the wearable monitor and/or a conformity of the wearable monitor to an ear shape of the wearer/user are accomplished via a pair of earpads, one or both of which may be flexible (e.g., silicone pads), and at least one of which is interchangeable with different sizes. In other words, at least one of the flexible earpads from the pair of flexible earpads can be removed from the wearable monitor and replaced with an earpad having different dimensions. In some such implementations, only one of the flexible earpads from the pair of flexible earpads is configured to be readily removable and replaced by the user. The removable earpad can be coupled, for example, to the second body portion via a ring-shaped structure thereof (also referred to herein as a “grommet” or “annular opening”), as discussed further below.

In some implementations, the at least one optical sensor is positioned within, and secured to, a cavity defined within a surface of one of the earpads (e.g., a flexible earpad that is coupled to the first body portion, and optionally not configured to be readily removed/replaced by the user, or a non-flexible earpad that is optionally not configured to be readily removed/replaced by the user). The at least one optical sensor can be “potted” in a substance such as an epoxy (e.g., an optically transparent epoxy, such that optical signals are not interfered with/altered, or are minimally interfered with/altered).

In some implementations, wearable monitors of the present disclosure include a flexible conductor (e.g., having a substantially S-shaped or curved/serpentine “ribbon” shape) such that at least a sub-assembly of the wearable monitor can “flex” to accommodate the process of positioning the wearable monitor on the ear of the user/wearer. Alternatively or in addition, wearable monitors of the present disclosure can include a DC power line communication transceiver chipset (e.g., Maxim MAX20340).

In some embodiments, a wearable monitoring kit includes an ear-wearable device (“wearable monitor”), a charger case, and optionally a set of flexible earpads configured to be interchangeably attached to the ear-wearable device via a grommet, as shown and described herein. Each flexible earpad from the set of flexible earpads can have a different set of associated dimensions, such that a “fit” of the car-wearable device can be adjusted/customized for different user (ear) sizes/geometries. Improving the fit of the ear-wearable device relative to the user/wearer can result in improvements to optical signal quality during operation of the ear-wearable device, as well as improvements to user comfort and ergonomics. In some such implementations, the charger case and the ear-wearable device are configured to exchange data and power (e.g., simultaneously) via one or more (e.g., two) charging contacts. Although described above as being “flexible” for purposes of conforming to a user's ear, the earpads can alternatively be formed from inflexible or substantially inflexible materials, for example for applications calling for greater durability/ruggedization (e.g., for underwater use, military use, etc.).

In some embodiments, during manufacture/assembly of a wearable monitor, an optical board (i.e., a circuit board including at least one optical sensor and/or at least one optical detector) is bonded to an ear pad using silicone (e.g., optically-clear silicone) or another material such as epoxy, thereby producing at least a sub-assembly (i.e., the optical board and the earpad) that is waterproof and that provides optical transparency sufficient for acceptable performance of the at least one optical sensor and/or at least one optical detector.

In some embodiments, wearable monitors of the present disclosure can include an alert mechanism such as a button or touchscreen that, when interacted with by a user, initiates/activates one or more processes (e.g., stored in a memory of the wearable monitor and executable via a processor (e.g., a microprocessor) of the wearable monitor). The one or more processes can include an emergency plan. The emergency plan can include, but is not limited to, one or more of: contacting emergency services (e.g., initiating a telephone call to 911), sending a short message service (SMS) message (i.e., a text message) alert to a pre-programmed phone number (e.g., to a mobile device of the user or other designated person), emitting a sound from a sound emitter of the wearable monitor (e.g., an electronic beep sound effect emitted via a speaker), a vibration generated by a haptic feedback element of the wearable monitor (e.g., a piezoelectric transducer), transmitting (e.g., via a transceiver onboard the wearable monitor) a signal to a mobile device to cause an alert such as a sound effect and/or a vibration, etc.

In some embodiments, a wearable monitor is configured to dial 911 (or other emergency service) in response to button press (or other interaction with an alert mechanism) made by a wearer/user, for example as part of a defined emergency plan. The wearable monitor can also include a speaker and microphone such that the monitor functions as a headset. For example, the wearer/user can hear the voice of the emergency response dispatcher via the speaker of the wearable monitor, and the wearer/user can speak to the emergency response dispatcher via the microphone of the wearable monitor. In some implementations, in response to the button press and in addition to dialing 911, the wearable monitor can be configured to concurrently trigger the generation and sending (e.g., via a wireless communication channel) of an alert text message to one or more emergency contact numbers (e.g., three separate emergency contact numbers) stored in a memory of the wearable monitor and/or accessible by the wearable monitor via a mobile software application thereof. The alert text message(s) can include one or more of: an alert message, vital signs/biometrics of the wearer/user, and an indication that 911 has been called.

In some embodiments, a wearable monitor is in the form of a wearable, hardware-based earpiece that is sized and shaped to fit and be worn about a portion of a wearer's ear (e.g., a helix, scapha, pinna, or any other portion of the external ear). The earpiece can clip onto, mechanically attach to, or otherwise grip the portion of the ear. For example, the earpiece include a gap or recess, defined therein, that is sized and shaped to receive the portion of the ear. When the ear portion is inserted into or received by the gap or recess, the earpiece can be configured to exert a bias or spring force that provides a squeezing action about the ear portion, such that the earpiece is securely retained on the wearer's ear. The wearable monitor can include one or more of: one or more light-emitting diodes (LEDs), one or more photosensors/photodetectors, one or more lightweight, power-efficient, wireless sensors (e.g., temperature sensor(s), pressure sensor(s), accelerometer(s), GPS sensor(s), voltage sensor(s), gyroscope sensor(s), photoplethysmogram (PPG) sensor(s), motion detector(s), electromagnetic sensor(s), photoacoustic sensor(s), piezoelectric sensor(s) etc.), a speaker, a microphone, a processor and a memory operably coupled to the processor. The memory stores instructions executable by the processor during operation. During operation, the one or more LEDs (e.g., red and/or green LEDs) can emit light through the portion of the ear, and the light transmitted through the portion of the ear can be detected at the one or more photosensors/photodetectors. One or more biometrics or vital signs can then be calculated (e.g., blood oxygen level, blood oxygen saturation (SpO2), heart rate, body temperature, pulse rate, respiration rate, blood pressure, hydration, VO₂ max (maximum rate of oxygen consumption attainable during physical exertion), electrocardiogram (EKG/ECG), NO₂ (nitric oxide levels), CO (carbon monoxide levels), perfusion index (PI), cardiac output/rhythm, etc.) based on the amount of light that is detected at the one or more photosensors/photodetectors and/or based on an amount of light that is absorbed by the ear (and, thus, does not reach the one or more photosensors/photodetectors). For example, blood oxygen saturation (SpO2) can be calculated based on the amount of light that is absorbed by the ear and using Beer's law (also referred to as the Beer-Lambert law, which states that absorbance is proportional to the concentration of one or more attenuating species in a material sample). In some implementations, an accuracy of the determination of the blood oxygen saturation increases with the thickness of the portion of the ear on which the wearable monitor is positioned during operation.

In some embodiments, the one or more LEDs include two LEDs—a first LED being a red (650 nm) LED and a second LED being an infrared (950 nm) LED. During operation, when light from each of the two LEDs passes through an adjacent portion of the ear, light emitted from the first (red) LED is partially absorbed by deoxyhemoglobin of the portion of the ear, and light emitted from the second (infrared) LED is partially absorbed by oxyhemoglobin of the portion of the ear (the amounts of which can be determined based on the detected light at the photodetector(s)/photosensor(s)). An oxygen concentration can then be calculated/detected, e.g., based on the ratio between the amount of light absorbed by deoxyhemoglobin and the amount of light absorbed by oxyhemoglobin. In some embodiments, the one or more LEDs includes at least one red and/or infrared LED for detecting a blood oxygen concentration of the wearer, and at least one green LED for detecting a pulse of the wearer. In some implementations, the determination of a blood oxygen concentration includes an adjustment to the detected signal (e.g., at the photodetector/photosensor) to correct for ambient or environmental light, such as sunlight. The adjustment can be based on an additional light sensor positioned, for example, on an outer surface of the wearable monitor. Such adjustments can be made, for example, when the wearable monitor is worn outdoors, in an environment with low lighting, and/or in an environment with a direct or indirect lighting source.

The memory can communicate with/via and/or store a software application that is compatible with one or more mobile devices (e.g., Windows, iOS, Android). The wearable monitor can be lightweight, power-efficient, and configured to communicate with one or more mobile devices and/or software application using one or more wireless communications protocols (e.g., Bluetooth®, 4G®, 5G®, etc.). The wearable monitor earpiece can include a power source that is rechargeable by a wired or wireless charging pod. The charging of the earpiece can occur when the earpiece is received at least partially within the charging pod and, optionally, when in electrical contact therewith.

In some embodiments, an emergency plan is activated in response to the wearer interacting with (e.g., pressing, tapping, sliding, etc.) the alert mechanism a predetermined number of times (e.g., once, twice, three times, four times, etc.) and/or with a predetermined frequency (e.g., three rapid taps within 1-5 seconds of each other). For example, a wearer pressing a button on the wearable monitor three times can trigger implementation/deployment of the emergency plan.

In some embodiments, the emergency plan can be executed through multiple communication channels to ensure reliability. For example, a wearable monitor may initiate a voice call using a cellular or voice over internet protocol (VOIP) while concurrently transmitting an SMS message to one or more predefined emergency contacts. In some implementations, the emergency plan includes a fail-safe mechanism that attempts alternative communication methods, such as Bluetooth® or Wi-Fi, when primary cellular connectivity is unavailable. Additionally, the emergency plan may trigger audible and haptic alerts on the wearable device and a paired mobile application to confirm activation. In some embodiments, the emergency plan workflow includes prioritization logic to determine the sequence of actions, such as contacting emergency services before notifying secondary contacts.

In some embodiments, a wearable monitor (also referred to herein as a “wearable device”) is configured to communicate (e.g., via wireless network communication) with a software application running on a mobile device (e.g., a smartphone, tablet, laptop computer, etc.) of a user/wearer of the wearable monitor or other individual. The software application can include code to cause storage of all vital records (e.g., blood oxygen level, heartrate/pulse, body temperature, hydration level, etc.) detected by one or more sensors onboard the wearable monitor, for example so that they can be sent to or shown to a medical provider. Alternatively or in addition, the software application can facilitate the definition/setting/customization, e.g., by a wearer/user of the wearable monitor or other authorized individual, of one or more set points or thresholds. The one or more set points or thresholds can include oxygen levels that will trigger an alert or alarm. Alternatively or in addition, the software application can facilitate the definition/setting/customization, e.g., by a wearer/user of the wearable monitor or other authorized individual, of one or more emergency contact telephone numbers to which an SMS message will be sent and/or that will be called when an alert/alarm is triggered.

In some embodiments, a wearable monitor includes a processor and a memory operably coupled to the processor. The memory stores instructions executable by the processor during operation. The instructions can include instructions to calculate an oxygen concentration, for example continuously and/or at predetermined intervals of time (e.g., every second, every 2 seconds, every 3 seconds, every 4 seconds, every 5 seconds, every 6 seconds, every 7 seconds, every 8 seconds, every 9 seconds, every 10 seconds, every 11 seconds, every 12 seconds, every 13 seconds, every 14 seconds, every 15 seconds, every 16 seconds, every 17 seconds, every 18 seconds, every 19 seconds, every 20 seconds, every 21 seconds, every 22 seconds, every 23 seconds, every 24 seconds, every 25 seconds, every 26 seconds, every 27 seconds, every 28 seconds, every 29 seconds, every 30 seconds, every 45 seconds, every minute, every 5 minutes, every 10 minutes, every 15 minutes, every 30 minutes, etc.).

The time interval can be configurable by a wearer/user of the wearable monitor and/or another authorized user, for example via a software application running on a mobile device of that individual and via wireless communication with the wearable monitor. In some implementations, the instructions include instructions to compare measured oxygen concentration levels (as measured by the wearable monitor) with a predetermined threshold value stored within the memory of the wearable monitor. The predetermined threshold value can be configurable by a wearer/user of the wearable monitor and/or another authorized user, for example via the software application.

In some embodiments, a wearable monitor is configured to emit an alarm sound and/or vibration in response to detecting that an oxygen level of a wearer/user is lower than a defined threshold value (e.g., representing an “alarm state”). An intensity, volume and/or frequency of the alarm sound and/or vibration can increase over time until the alarm is acknowledged by the wearer/user (e.g., via interaction of the wearer/user with an alert mechanism of the wearable monitor or via a graphical user interface (GUI) rendered by a software application of a mobile device of the wearer/user. Alternatively or in addition, an intensity, volume and/or frequency of the alarm sound and/or vibration can increase with and/or in proportion to an increase in a calculated difference between the detected oxygen level and the defined threshold value, such that the increasing intensity, volume and/or frequency of the alarm sound and/or vibration represents an increasing severity of the alarm state. Similarly, the intensity, volume and/or frequency of the alarm sound and/or vibration can decrease with and/or in proportion to a decrease in a calculated difference between the detected oxygen level and the defined threshold value, such that the increasing intensity, volume and/or frequency of the alarm sound and/or vibration represents a decreasing severity of the alarm state. The wearable monitor can terminate the alarm sound and/or vibration upon detection that a current oxygen level is equal to or greater than the defined threshold value.

In some embodiments, oxygen levels detected by (and, optionally, other sensor data gathered by/detected at) the wearable monitor are stored locally (e.g., within a memory of the wearable monitor) and/or are transmitted (e.g., via a transceiver of the wearable monitor) to a cloud-based server or other storage repository, for example using a software application. The wearable monitor, the cloud-based server and/or a software application associated with the wearable monitor can be configured to analyze sensor data collected/detected at the wearable monitor, for example to determine one or more conditions or biometric parameters based on the sensor data, to detect patterns associated with the sensor data over time, etc. Data stored locally and/or transmitted can include, in addition to the detected oxygen levels and optional other sensor data, information such as time and date of detection events associated with such data, an identifier of the earpiece, an identifier associated with the wearer, etc. Data stored at the cloud-based server can be downloaded therefrom by the wearer/user and/or other authorized person, for example to show a physician for purposes of diagnosis, investigation of anomalous events, etc. The ability of the wearer/user to download data from the cloud-based server can be limited, for example, to daily or weekly. Alternatively or in addition, a user may send a request to the cloud-based server, the request including a query specifying a range of dates for which he/she would like to retrieve data. Although described herein as pertaining to oxygen levels, systems and methods of the present disclosure can, alternatively or in addition, be used to detect other biometrics or vital signs, such as heartrate/pulse, body temperature, hydration level, salt level, etc.

In some embodiments, a mobile software application is configured for use with one or more wearable monitors of the present disclosure. The mobile software application can be compatible with one or more of Android, iOS and Windows, and can facilitate continuous communication between one or more mobile devices running the mobile software application and a wearable monitor (e.g., via one or more wireless sensors of the wearable monitor). The mobile software application can be configured to record/store detected vitals/biometric information (e.g., continuously, periodically, intermittently, and/or upon request or user interaction therewith) and, optionally, upload the detected data to a cloud-based storage for future reference. The mobile software application can be configured to send and/or receive a signal to cause display, e.g., in a GUI of a mobile device of the wearer/user, of one or more of the detected data values (and/or graphical representations thereof, for example over time), for example including oxygen levels, heart rate, etc. The mobile software application can be configured to identify and/or cause storage (e.g., in a database or other repository) of geometrics (e.g., geographic data such as GPS data) and/or barometrics (e.g., environmental data such as barometric pressure) in addition to the biometric information detected by the wearable monitor(s), and/or to track the occurrence of acute/emergency events over time (e.g., as indicated by the triggering of the emergency plan). Data received and/or stored by the software application can include crowd-sourced data (e.g., from multiple different wearable monitors associated with multiple different wearers, optionally without including identifying information associated with the individual wearers).

In some embodiments, data detected by one or more wearable monitors and information derived from such data is stored in a common repository and used to train a machine learning (ML) or artificial intelligence (AI) algorithm. The trained ML/AI algorithm can be used to predict future acute/emergency events (i.e., to perform predictive analytics), for example based on current (contemporaneous) sensor readings detected at the wearable monitor of a particular wearer. When a future acute/emergency event is predicted, an “early warning” alert can be generated and presented to a wearer/user (e.g., via a GUI of the wearer's mobile device, via the software application running thereon) such that the wearer/user can take remedial or preventative action (e.g., increase an oxygen intake).

In any of the foregoing embodiments, the wearable health monitor of the present disclosure can further include a PPG sensor. In some embodiments, the PPG sensor is configured to measure volume changes in blood. In some embodiments, the PPG sensor may include one or more LEDs configured to emit light at a tissue site, and a photodiode configured to measure the intensity of the non-absorbed light reflected from the tissue. In some embodiments, the one or more LEDs can include at least one of a red LED, a green LED, or a yellow LED. Ear wearable PPG sensors may exhibit enhanced reliability compared to other PPG sensors positioned on the other parts of a body. The anatomical structure of the ear can provide a stable and less mobile environment, thereby diminishing the likelihood of motion artifacts during monitoring. Additionally, the thinner skin in the ear can facilitate deeper light penetration, which may enhance the accuracy of PPG measurements. Furthermore, the vascular configuration within the ear is conducive to capturing changes in blood volume, establishing it as an optimal (or desirable) site for PPG sensor deployment.

In some embodiments, the PPG sensor may be configured to detect electrocardiogram (EKG) signals. In some embodiments, the PPG sensor may operate in communication with other sensors (e.g., a gyroscope, an accelerometer, a pulse oximeter) included in the wearable monitor to measure the EKG signals. That is, in some embodiments, the wearable monitor can provide EKG signals of a wearer based on data provided by a plurality of sensors including the PPG sensor. Accordingly, the PPG sensor can provide data regarding cardiac activity, thus augmenting the overall utility of the wearable monitor.

In any of the foregoing embodiments, the wearable monitor can further include an accelerometer. In some embodiments, the accelerometer can be configured to measure heart rate variability (HRV). The determination of HRV can provide insights into the user's overall cardiovascular health. The HRV can be calculated by analyzing the time variation between heartbeats using the accelerometer. Accelerometers typically include a sensing element configured to convert mechanical motion into electrical signals, allowing the accelerometer to detect changes in acceleration along multiple axes such as the X, Y, and Z axes. In some embodiments, the accelerometer can include a piezoelectric crystal as the sensing element. In some embodiments, the accelerometer can include a capacitive sensor as the sensing element. In some embodiments, the accelerometer can operate in conjunction with a pulse rate sensor or a heart rate monitor, facilitating the collection of synchronized data regarding the time intervals between heartbeats.

In some embodiments, the accelerometer can be configured to assess body position and detect falls alternative to or in combination with measuring HRV. By measuring changes in acceleration and orientation, the accelerometer can determine whether the user is standing, sitting, or lying down. This capability is particularly advantageous for fall detection, as the wearable monitor can identify sudden, abnormal movements indicative of a fall event. For instance, an unexpected decrease in vertical acceleration may signal a fall, triggering an alert to notify caregivers or emergency services. In some embodiments, accelerometer may monitor head motion.

In some embodiments, the body position and the head position of a wearer of the monitor can be determined by the accelerometer together with a gyroscopic sensor disposed within the wearable monitor. The integration of the gyroscopic sensor may further improve the wearable monitor's ability to assess body dynamics.

In some embodiments, the accelerometer can be configured to capture the direction of blood motion, enabling a PPG sensor to mitigate movement artifacts, particularly during periods of intense physical activity. The integration of such sensor data may yield more accurate readings of EKG. In some embodiments, the wearable device can employ sensor fusion algorithms (e.g., multimodal integration) to combine data from a PPG sensor and an accelerometer to mitigate motion artifacts during high-activity periods. For example, accelerometer data may be used to identify and compensate for abrupt movements that could distort optical readings, thereby improving the accuracy of SpO₂ and heart rate measurements. In some embodiments, the sensor fusion algorithm operates in real time and dynamically adjusts sampling rates based on detected activity levels. This capability can enable accurate monitoring during exercise, sleep, and other scenarios where motion artifacts are prevalent.

In any of the foregoing embodiments, the wearable monitor of the present disclosure may further include a gyroscopic sensor. In some embodiments, the gyroscopic sensor is a microelectromechanical (MEMS) gyroscope sensor. In some embodiments, the gyroscopic sensor is configured to detect angular velocity and monitor the orientation or movement of the wearable monitor. The gyroscopic sensor may be used in conjunction with an accelerometer to enhance the accuracy of activity tracking, including recognizing specific movements or physical activities performed by the user.

In some embodiments, the gyroscopic sensor is operatively connected to the mobile software application of the wearable monitor and is configured to provide real-time data on the rotational movement of the wearable monitor. This data may be utilized to detect posture, balance, or sudden changes in movement, such as falls. In some embodiments, the mobile software application is configured to use this information to adjust sensor readings or trigger alerts when irregular motion patterns are detected.

The gyroscopic sensor may also assist in refining the measurement of other physiological parameters by accounting for wearer movement. For instance, during physical activity, the gyroscopic sensor may help to differentiate between voluntary movements and involuntary tremors or other anomalies, allowing for more precise monitoring of blood oxygen levels or other biometric data. In some embodiments, the combination of data obtain from the gyroscopic sensor and an accelerometer may enhance the detection of conditions related to mobility or movement disorders, thereby providing additional health insights.

Accordingly, the wearable monitors of the present disclosure can be used to monitor balance or detect issues like fall, instability in elderly users or those with disorders that can cause sudden falls (e.g., acute mountain sickness (AMS)).

In any of the foregoing embodiments, the wearable monitor of the present disclosure may further include one or more photodetector(s) and LED(s) configured to measure blood gases, such as carbon monoxide (CO). In some embodiments, the photodetectors are configured to detect light at multiple wavelengths, allowing the device to distinguish between different hemoglobin states, including oxygenated hemoglobin (HbO2), deoxygenated hemoglobin (Hb), and carboxyhemoglobin (HbCO).

In any of the foregoing embodiments, the wearable monitor described herein may also include a temperature sensor. In some embodiments, this temperature sensor can be configured to measure the wearer's skin temperature. Outputs (e.g., readings) from some of the aforementioned sensors (e.g., a PPG sensor, a pulse oximeter) can be affected by temperature fluctuations. Therefore, real-time temperature data can be used to adjust the accuracy of these readings.

In any of the foregoing embodiments, the wearable monitor of the present disclosure may further include a non-invasive glucose sensor configured to determine blood sugar level. In some embodiments, the non-invasive glucose sensor can be selected from at least one of an optical sensor, a photoacoustic sensor, an electromagnetic sensor, an electrochemical sensor, or a bioimpedance sensor.

In any of the foregoing embodiments, the wearable monitor provided herein may further include a proximity sensor configured to detect the positioning of the wearable monitor relative to the skin and/or the ear, ensuring optimal contact for accurate measurements.

In any of the foregoing embodiments, the wearable monitor may further include an ambient light sensor. In some embodiments, the ambient light sensor is configured to adjust the intensity of an LED output depending on the external lighting conditions, minimizing interference from environmental light and improving the signal quality resulting from the LED output.

In any of the foregoing embodiments, the wearable monitor may further include a battery voltage sensor that reduce power consumption to a desired level by regulating the operation of sensors, such as pulse oximeters, PPG sensors, based on wearer's activity. For example, during periods of low activity (e.g., during sleep), the wearable monitor may enter a low-power mode while still tracking critical health metrics, thereby extending battery life without sacrificing data accuracy.

In any of the foregoing embodiments, the wearable monitor described herein can further include one or more of the aforementioned sensors configured to generate a sleep report based on outputs such as heart rate variability (HRV), respiratory patterns, and body movements (e.g., sleep movements). The data collected during sleep can be used to assess sleep quality and duration.

In some embodiments, at least two of the aforementioned sensors such as a PPG sensor, an accelerometer, a gyroscopic sensor, a pulse oximeter, or a temperature sensor, can be used in combination to measure blood pressure and/or improve the accuracy of blood pressure estimation. For example, a PPG sensor may measure the time intervals between heartbeats by capturing pulse wave signals, while an accelerometer and/or a gyroscope detects the wearer's body position and movement. This combined data can be processed using an algorithm to estimate pulse transit time (PTT) or pulse wave velocity (PWV), which correlate with blood pressure levels.

In some embodiments, the wearable monitors described herein may be configured to monitor heart rate recovery (HRR) and respiratory rate using any combination of the aforementioned sensors.

In some embodiments, the wearable monitors described herein may be configured to monitor SpO₂ entropy or variability, providing insights into the stability of blood oxygen levels over time using any combination of the aforementioned sensors.

In some embodiments, the wearable monitors described herein may be configured to calculate a perfusion index (PI), which is the ratio of pulsatile blood flow to non-pulsatile or static blood flow in peripheral tissue, using any combination of the aforementioned sensors.

By integrating SpO₂ variability and the perfusion index, the wearable monitor can offer a more detailed analysis of the user's cardiovascular and respiratory health, particularly under varying conditions such as physical exertion or rest.

In any of the foregoing embodiments, the wearable health monitor of the present disclosure may include a first LED and a secondary infrared (IR) and/or red LED. At least a portion of the light emitted by the first LED may be configured to be detected by a first photodetector, and at least a portion of the light emitted by the secondary IR and/or red LED may be configured to be detected by a second photodetector. In some embodiments, the first and second photodetectors may be the same detector, while in other embodiments, the first and second photodetectors may be separate detectors. In further embodiments, the first LED and/or the secondary IR and/or red LED may include a plurality of LEDs.

In some embodiments, the first LED and/or the secondary IR and/or red LED may be configured to detect, measure, or estimate one or more vital signs, biometric parameters, conditions (e.g., falls), or diseases (e.g., acute mountain sickness (AMS), Alzheimer's disease, dementia), as described in the various embodiments of the present disclosure. In any of the foregoing embodiments, the wearable monitor may be further configured to measure environmental parameters (e.g., temperature, humidity, pressure, altitude, and/or the like) in real-time. The wearable monitor can include one or more environmental sensors configured to continuously, semi-continuously or intermittently detect at least one of altitude, humidity, air quality, or barometric pressure. In some embodiments, the wearable monitor may include calibration routines for the one or more environmental sensors to maintain accuracy under varying conditions. For example, altitude sensors may be calibrated using barometric pressure data and GPS readings to account for environmental fluctuations. In some embodiments, humidity and temperature measurements can be used to adjust SpO2 calculations, compensating for physiological changes induced by environmental factors. A use case includes high-altitude sports or aviation scenarios, where simultaneous monitoring of oxygen saturation and altitude enables detection of hypoxia risk. The integration of environmental and physiological data can provide a comprehensive health assessment, enhancing safety during extreme conditions.

Real-time measurements of environmental parameters together with vital signs or biometric data can facilitate a comprehensive assessment of the user's physiological condition, thereby enabling more precise health and safety monitoring. For instance, simultaneous monitoring of blood oxygen levels and altitude can aid in identifying health risks associated with changes in altitude. A decrease in blood oxygen concentration at elevated altitudes could trigger alerts, indicating a potential need for intervention. Furthermore, this integration enhances the monitor's capacity to adapt to varying environmental conditions, improving the accuracy of health assessments and ensuring timely responses to emergencies.

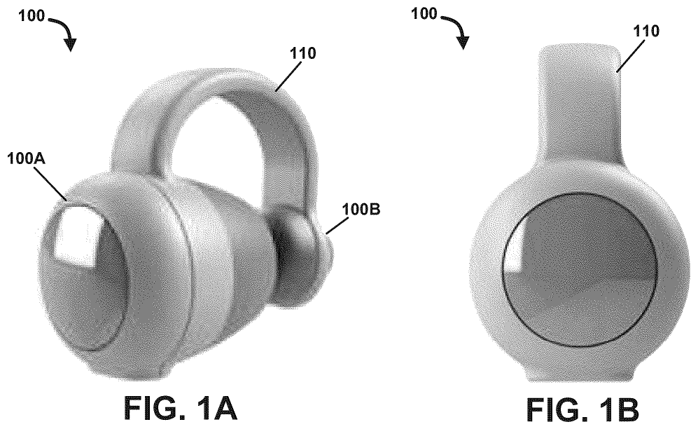

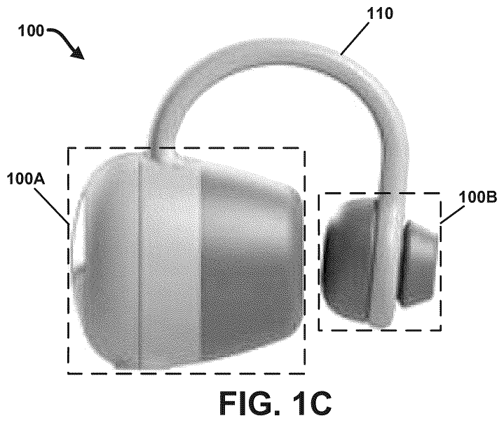

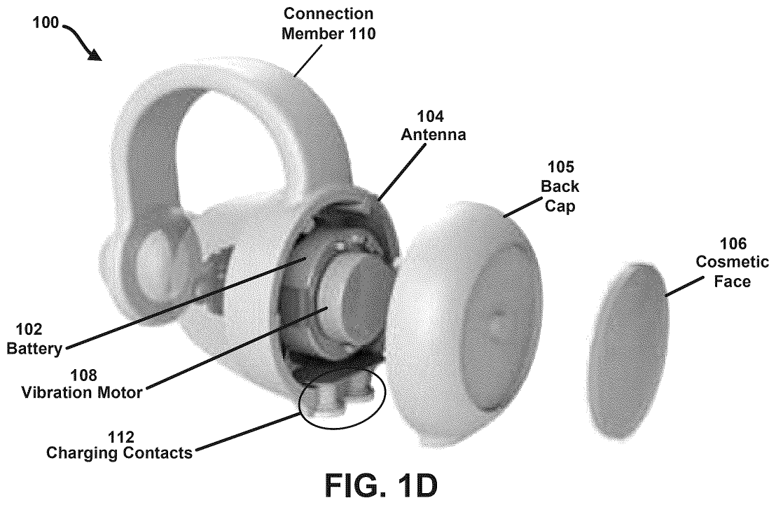

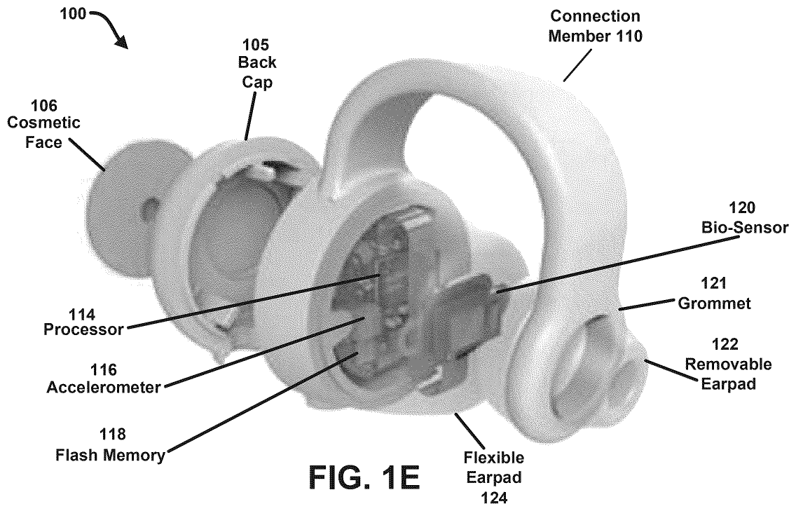

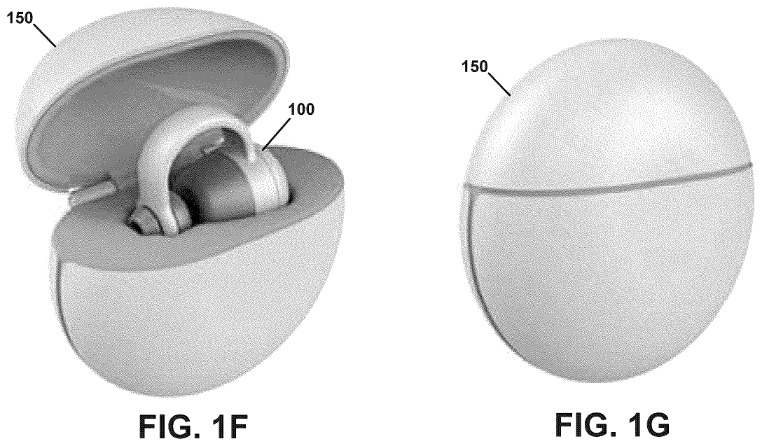

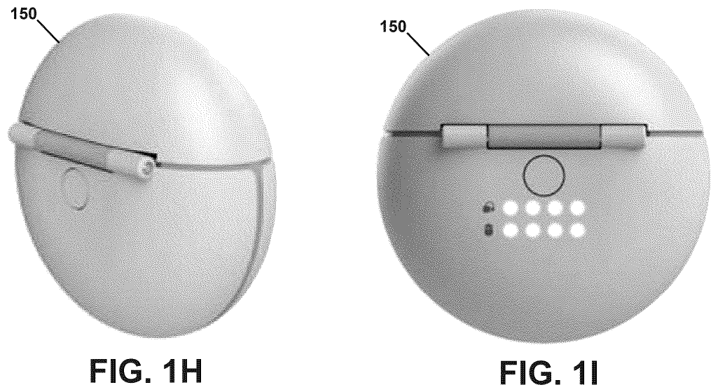

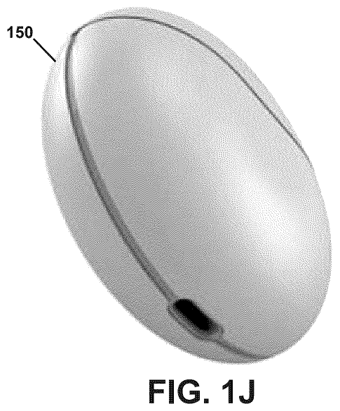

FIGS. 1A-1C are renderings of a wearable monitor 100, configured to be worn about a portion of an ear of a user, according to some embodiments. As shown in FIGS. 1A-1C, the wearable monitor 100 includes a first body portion 100A and a second body portion 100B, the first body portion 100A and the second body portion 100B mechanically coupled by a connection member 110. At least a portion of the first body portion 100A and the second body portion 100B may be integrally formed or monolithically formed with one another, for example from nylon, plastic, or another suitable material.

FIGS. 1D-1E are exploded-view renderings of the wearable monitor 100 of FIGS. 1A-1C, showing components thereof. For example, as shown in FIG. 1D, the wearable monitor 100 includes a battery 102, antenna 104, vibration motor 108, charging contacts 112, a back cap 105, and a cosmetic face 106. Each of the battery 102, antenna 104, vibration motor 108, charging contacts 112, back cap 105, and cosmetic face 106 can be contained within and/or attached to the first body portion (i.e., 100A of FIGS. 1A-1C). FIG. 1E is a view from a different perspective relative to FIG. 1D, and shows additional components of the first body portion-namely a processor 114, an accelerometer 116, a flash memory 118, a bio-sensor 120, and a flexible earpad 124. FIG. 1E also shows that the second body portion includes a grommet 121 and a removable earpad 122 positioned within the grommet 121.

FIGS. 1F-1J are renderings of the wearable monitor of FIGS. 1A-1C and a charger case 150 for the wearable monitor of FIGS. 1A-1C. The charger case 150 has a lower/base portion and a lid portion, the base and lid portions coupled to one another by a hinge. As shown in FIG. 1F, the wearable monitor 100 fits within a complementary-shaped recess in the charger case 150, and is partially received therein. In other words, when positioned inside the charger case 150, a first portion of the wearable monitor 100 sits within the recess, and a second portion of the wearable monitor 100 is positioned within a recess defined by the lid of the charger case 150. In some embodiments, the charger case 150 is water-resistant and/or waterproof. In some embodiments, the charger case 150 is dust resistant. In some embodiments, the charger case 150 has an ingress protection (IP) code, a part of the IEC 60529:2013 standard, of 67. In some embodiments, the charger case 150 has an IP code of 68. In some embodiments, the charger case 150 can be submerged in water up to a depth of 0.5 m, 1.0 m, or 1.5 m, for 5 minutes, 10 minutes, 30 minutes, or 1 hour. In some embodiments, the charger case 150 is configured to support wireless charging. In some embodiments, the charger case 150 does not need to be plugged in for charging; instead, it can be docked onto a charging station or placed on a wireless charger. This enables wireless charging of the wearable device without the need to remove it from the case 150.

In some embodiments, the charger case 150 can include a rechargeable battery having a capacity between about 500 mAh and 2000 mAh, sufficient to provide multiple charge cycles to the wearable device without requiring external power. The charger case 150 may be configured to achieve a full recharge within a time interval ranging from about 30 minutes to about 2 hours, depending on the charging protocol employed. In some embodiments, the charger case 150 includes safety features such as thermal cutoff circuits, overcharge protection, and/or short-circuit prevention to maintain operational integrity. A battery monitoring circuit may further provide real-time feedback on charge status via LED indicators or a graphical user interface of a paired mobile application.

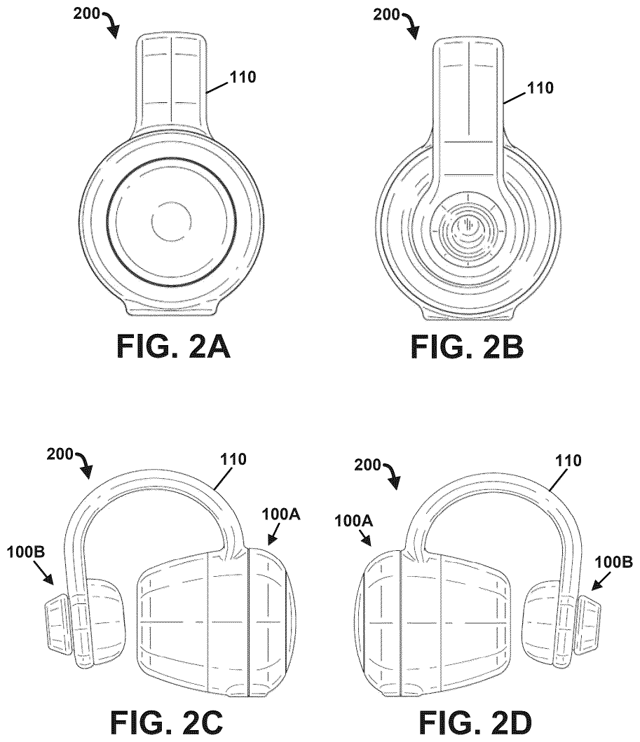

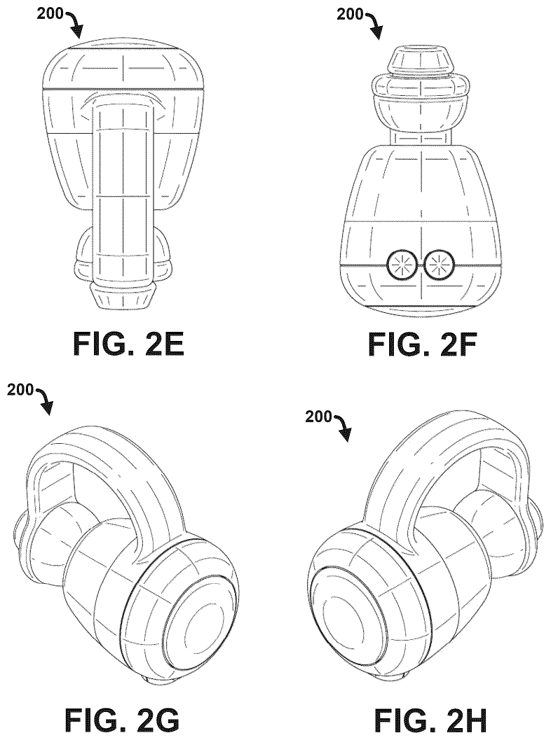

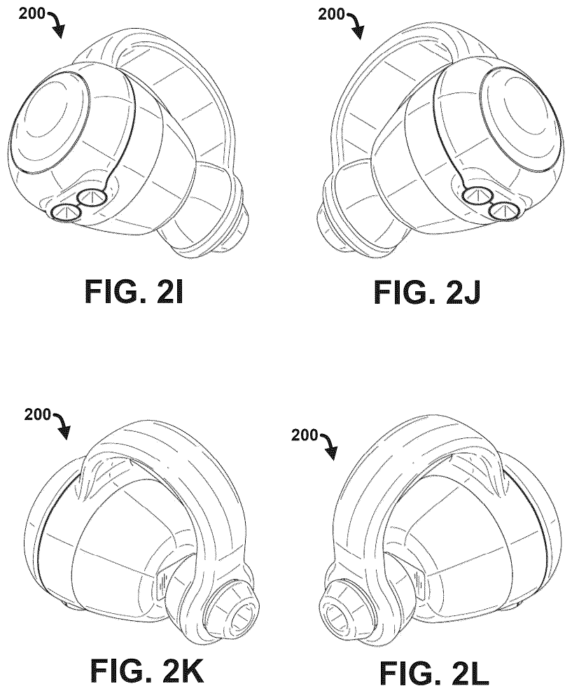

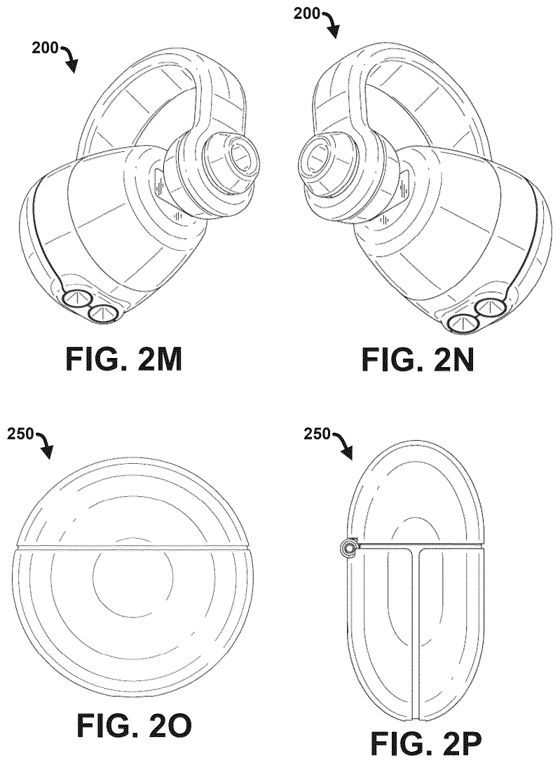

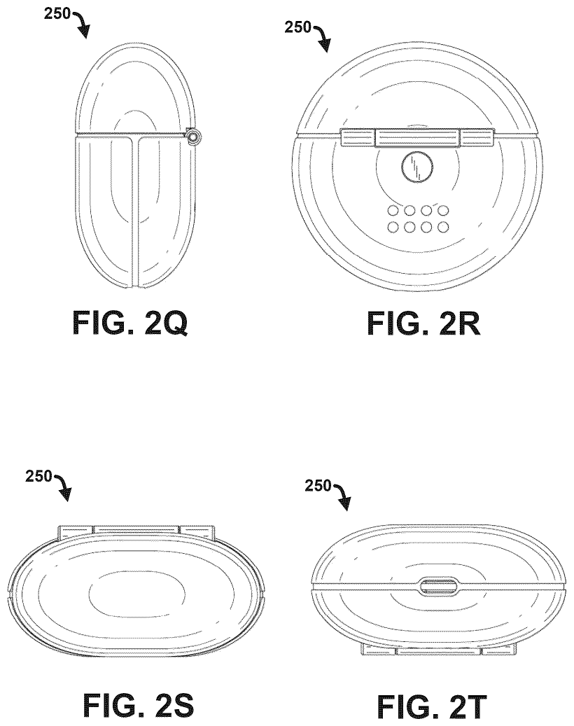

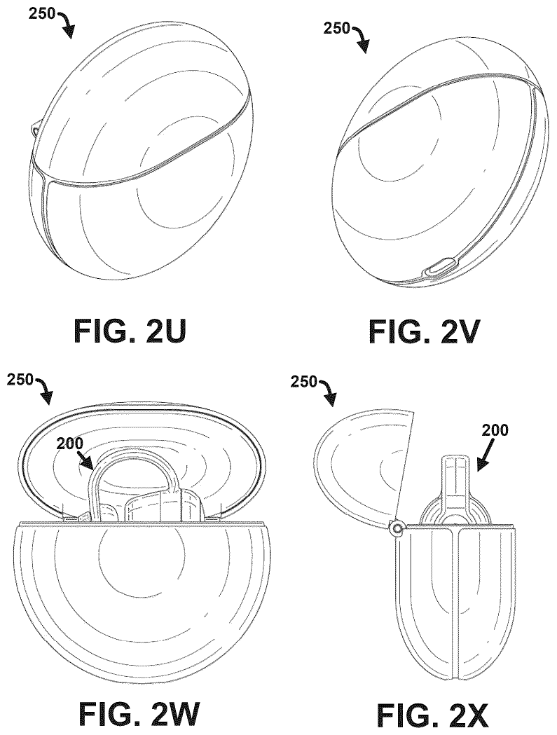

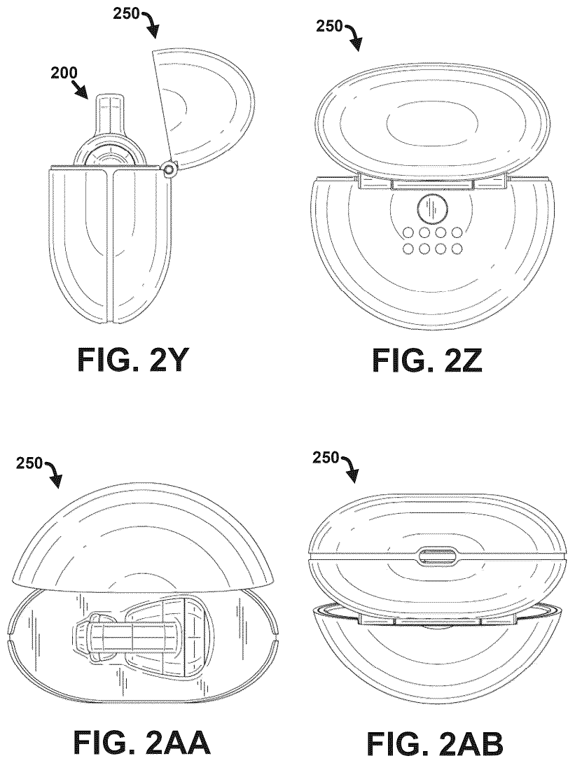

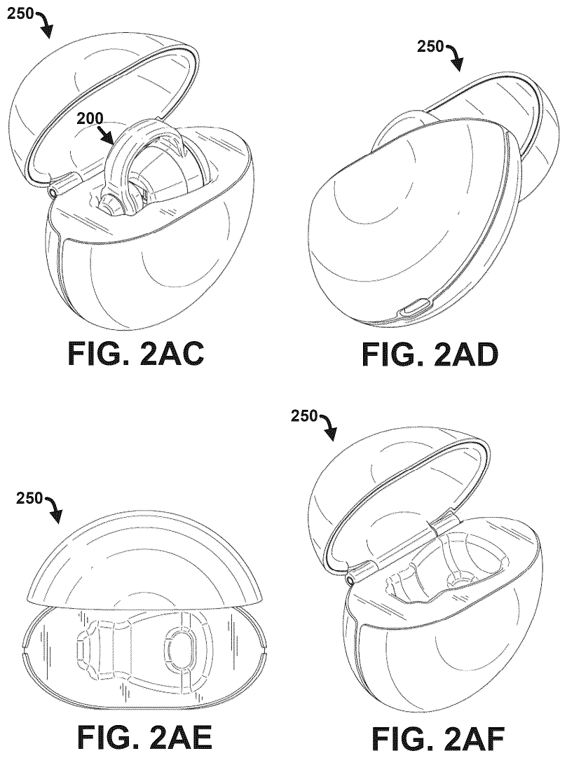

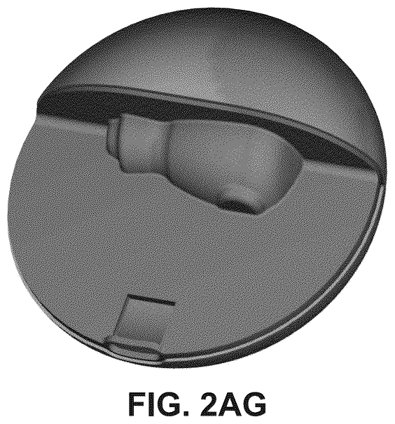

FIGS. 2A-2N are drawings of a wearable monitor 200, configured to be worn about a portion of an ear of a user, according to some embodiments. FIGS. 2O-2V are drawings of a charger case in a closed configuration, the charger case configured to contain, and to supply power to, the wearable monitor of FIGS. 2A-2N, according to some embodiments. FIGS. 2W-2AD are drawings of the charger case of FIGS. 2O-2V in an open configuration, the charger case containing the wearable monitor of FIGS. 2A-2N. FIGS. 2AE-2AF are drawings of the charger case of FIGS. 2O-2V in an open configuration and not containing a wearable monitor. FIG. 2AG is a rendering of a charger case with a panel removed to show an underlying shape of a recess/pocket configured to receive a wearable monitor, according to some embodiments.

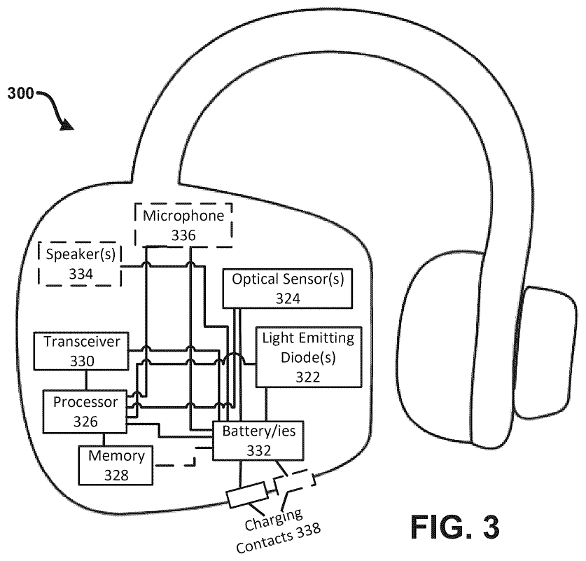

FIG. 3 is a schematic drawing showing components of a wearable monitor 300, according to some embodiments. As shown in FIG. 3, the wearable monitor 300 includes a memory 328, a processor 326, a transceiver 330, one or more batteries 332 or other power sources, one or more LEDs 322 (optionally in a row or array), one or more optical sensors 324, and optionally one or more speaker(s) 334 and/or a microphone 336. The one or more batteries 332 are electrically coupled to, and configured to supply power to, each of the processor 326, the transceiver 330, the LED(s) 322, the optical sensor(s) 324, the optional speaker(s) 334, the optional microphone 336, and optionally the memory 328. The processor 326 is operably coupled to each of the memory 328, the LED(s) 322, the optical sensor(s) 324, the transceiver 330, and the optional microphone 336.

The wearable monitor 300 optionally includes an alert mechanism (e.g., an actuatable button) that is electrically connected to the one or more batteries 332 and is operably coupled to at least one of the processor 326 and the transceiver 330. The transceiver 330 can be configured to send and/or receive communications (e.g., to/from a remote compute device such as a mobile device of a wearer/user of the wearable monitor 300 or other authorized person), for example including data gathered/detected by the one or more optical sensor(s) 324 and/or the processor 326, and/or stored in the memory 328. The processor 326 can be configured to control (e.g., based on processor-executable instructions stored in the memory 328 and/or received via the transceiver 330) at least one of the one or more LEDs 322, the microphone 336, the speaker 334, or the one or more batteries 332, via an electrical conduit (not shown). During operation, and when worn about a portion of an ear of the wearer/user, the one or more LEDs 322 emit light such that the emitted light is reflected by a portion (e.g., a surface or surface layer) of the ear in a direction toward the one or more optical sensors 324. The one or more optical sensors 324 detect a signal in response to the reflected light. In some implementations, the processor 326 determines an oxygen level of the wearer based on the detected signal and, optionally, stores the determined oxygen level in the memory 328, optionally with date information, time information, and/or other sensor data detected based on one or more other sensors (not shown) onboard the wearable monitor 300. Alternatively or in addition, raw data including the detected signal (optionally with data detected using the one or more other sensors) can be sent, via the processor 326 and using the transceiver 330, to a remote compute device such as a cloud-based server, a remote mobile device, etc., for determination of an oxygen level based on the raw data.

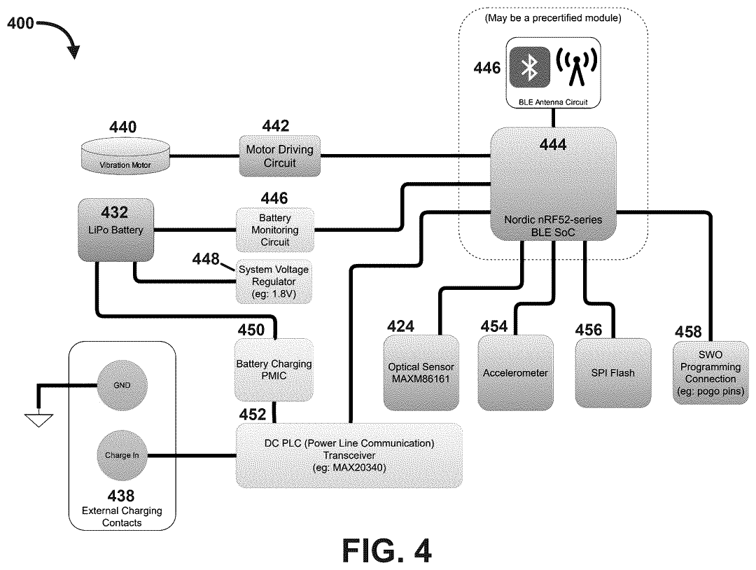

FIG. 4 is a block diagram showing components of a wearable monitor 400 (such as the wearable monitor shown in FIGS. 1A-1E and/or 2A-2N), according to some embodiments. As shown in FIG. 4, the wearable monitor 400 includes a vibration motor 440 operatively coupled to a motor driving circuit 442, a battery (e.g., lithium polymer (“LiPo”)) 432 operatively coupled to a battery monitoring circuit 446, a system voltage regulator (e.g., 1.8V) 448, and a battery charging power management integrated circuit (“PMIC”) 450. The battery charging PMIC 450 is operatively coupled to a direct current (“DC”) power line communication (“PLC”) transceiver (e.g., a MAX20340) 452. Each of the DC PLC transceiver 452, the battery monitoring circuit 446, and the motor driving circuit 442 is operatively coupled to a system-on-a-chip (“SoC”) (e.g., a Nordic nRF52-series) 444, which in turn is operatively coupled to an antenna (e.g., a Bluetooth® (“BLE”) antenna circuit). The DC PLC transceiver 452 is also operatively coupled to a “charge in” external charging contact from a pair of external charging contacts 438 which also includes a ground contact. The SOC 444 is operatively coupled to one or more optical sensors 424, an accelerometer 454, a serial peripheral interface (“SPI”) flash memory 456, and a serial wire output (“SWO”) programming connection (e.g., pogo pins) 458.

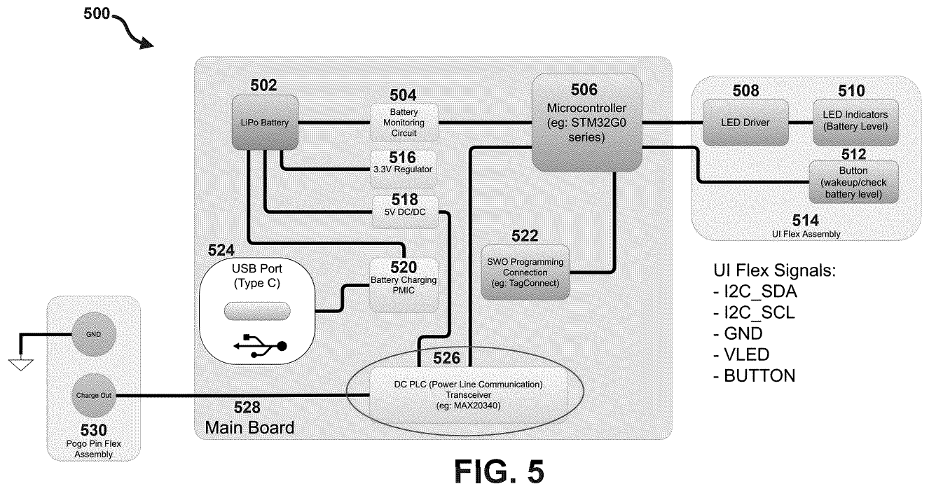

FIG. 5 is a block diagram showing components of a charger case 500 (such as the charger case shown in FIGS. 2O-2AF), according to some embodiments. As shown in FIG. 5, the charger case 500 includes a battery 502 (e.g., a LiPo battery), which is operatively coupled to each of a battery monitoring circuit 504, a regulator 516 (e.g., 3.3V), a DC-to-DC converter 518, and a battery charging PMIC 520. The battery monitoring circuit 504 is operatively coupled to a microcontroller 506 (e.g., a STM32G0 series microcontroller), which in turn is operatively coupled to each of a DC PLC transceiver 526 (e.g., a MAX20340), a SWO programming connection 522 (e.g., TagConnect), a LED driver 508, and a button 512 or other actuator (e.g., to trigger “wakeup” of the charger case and/or to check a battery power level). The LED driver 508 is operatively coupled to one or more LED indicators 510 (e.g., to indicate battery level, for example based on color of illumination and/or illumination status). The battery charging PMIC 520 is operatively coupled to a universal serial bus (USB) 524 (e.g., type C), and the DC PLC 526 is operatively coupled to a “charge out” contact from a pair of contacts 530, which also includes a ground contact. The pair of contacts 530 may be configured to mechanically mate with, and electrically couple to, the external charging contacts 438 of the wearable monitor 400 of FIG. 4.

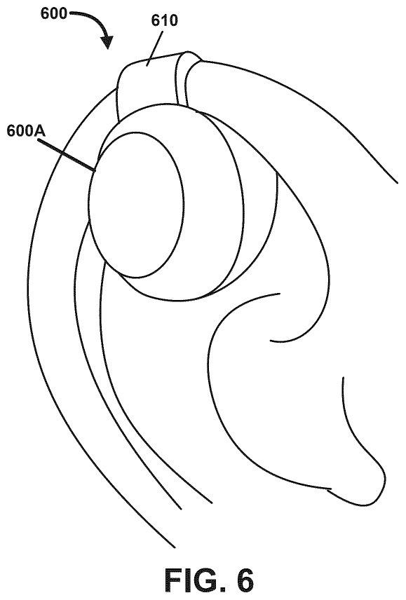

FIG. 6 is a drawing of a wearable monitor 600 when worn about an upper portion of an ear of a user/wearer, with a first body portion 600A and connection member 610 visible, according to an embodiment. The connection member 610 can be made of nylon, plastic, or any other suitable material. Although shown in FIG. 6 as being positioned about the upper portion of the ear, the wearable monitor 610 may alternatively be positioned on the ear lobe or on any other portion of the ear. For example, the wearable monitor 600 can be positioned about the helix, scapha, pinna, or earlobe of the ear. During placement or “donning” of the wearable monitor 600, a gap between the first body portion 600A and a second body portion of the wearable monitor 600 can be expanded by, e.g., by moving the first body portion and the second body portion away from one another, expanding the radius of curvature of the connection member 610, and/or deforming the connection member 610. Once positioned on the portion of the ear of the user (a “second configuration”), and external force(s) are removed from the wearable monitor 600, the first body portion 600A and the second body portion may naturally move a distance, equal to a portion of the gap, toward one another, e.g., by virtue of a shape memory or an inherent spring force (or bias force) of the connection member 610, such that the wearable monitor 600 remains securely positioned on the portion of the ear of the user during use (e.g., during movement of the user).

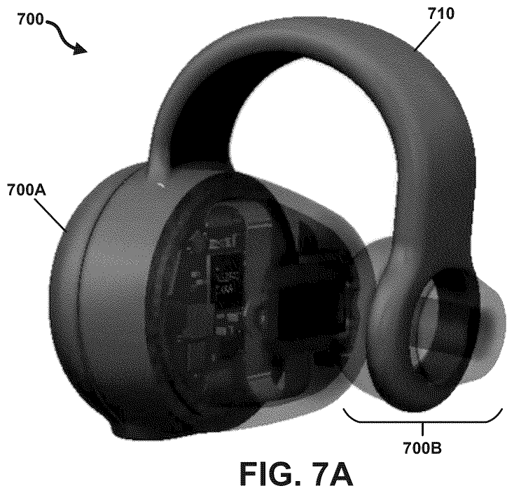

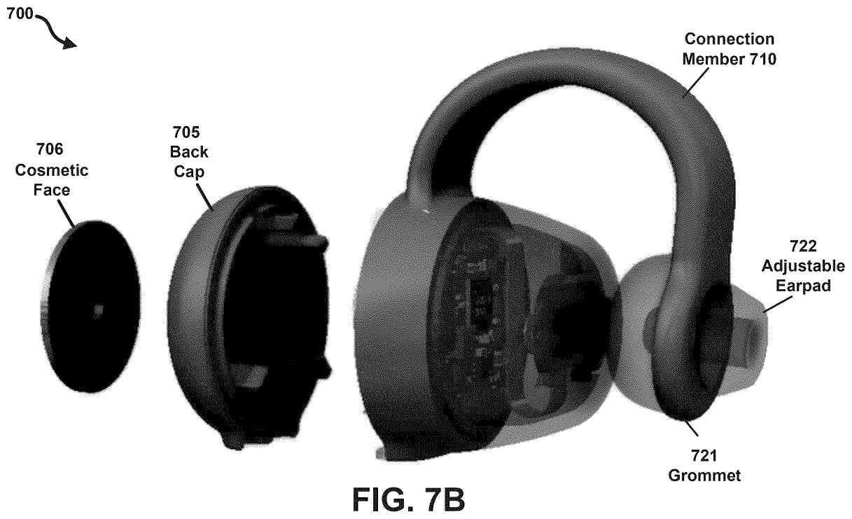

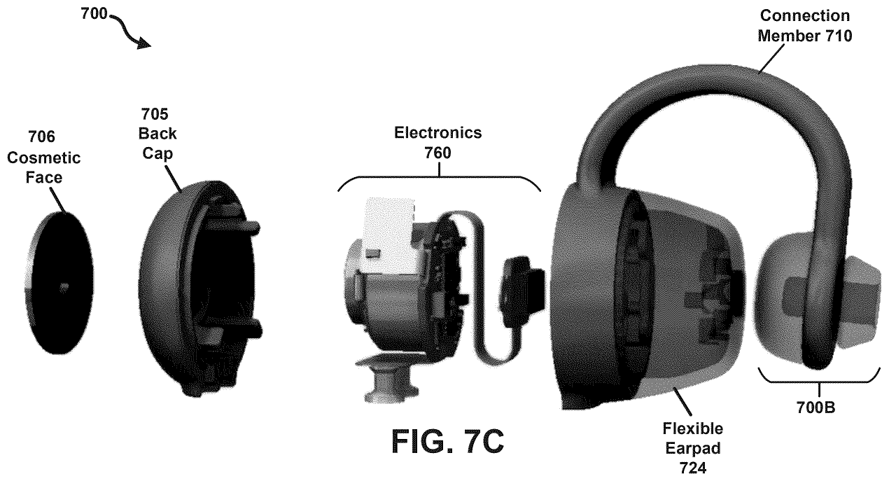

FIGS. 7A-7C are renderings of a wearable monitor 700, configured to be worn about a portion of an ear of a user, at various stages of assembly and showing components thereof, according to some embodiments. As shown in FIG. 7A, the wearable monitor 700 includes a first body portion 700A and a second body portion 700B, the first body portion 700A and the second body portion 700B mechanically coupled by a connection member 710. FIGS. 7B-7C show that the wearable monitor 700 includes a cosmetic face 706, a back cap 705, a connection member 710 connecting the first body portion and the second body portion 700B, electronics 760 (e.g., including some or all of the electronic components shown in FIGS. 3-4), a flexible earpad 724, a grommet 721, and an adjustable earpad 722.

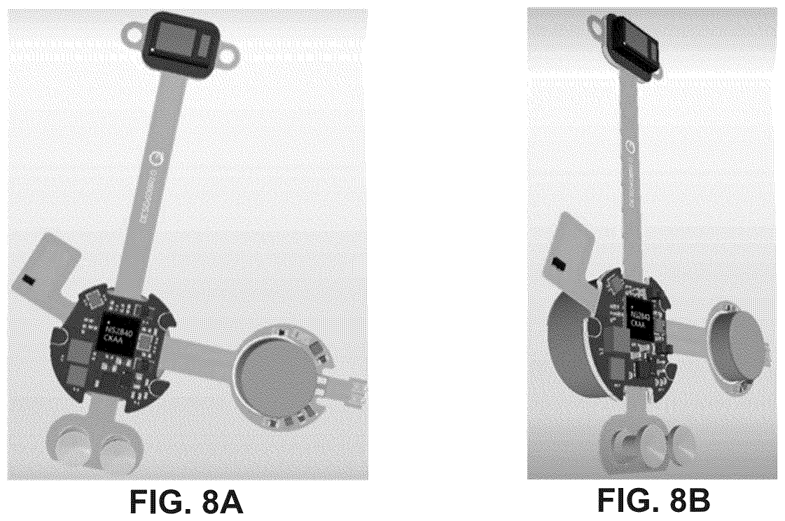

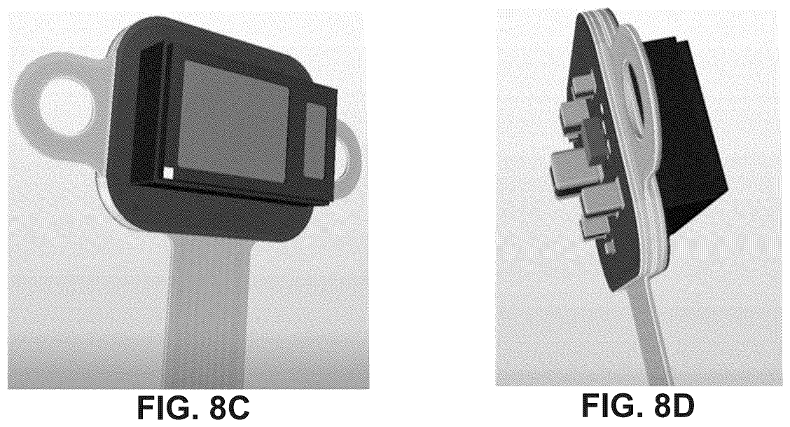

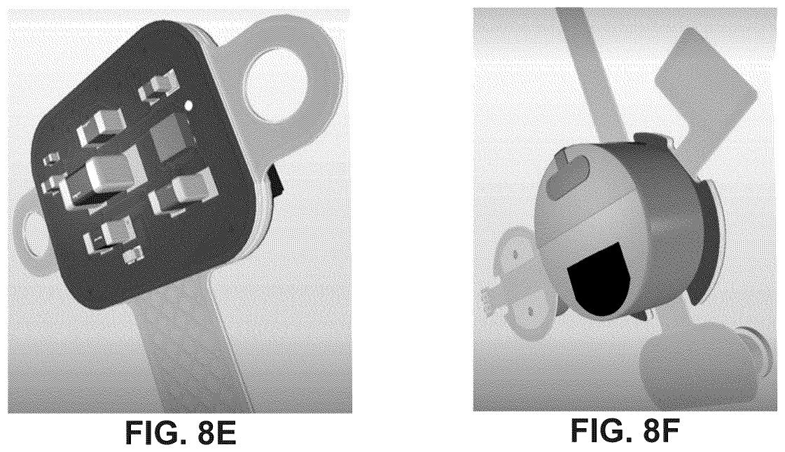

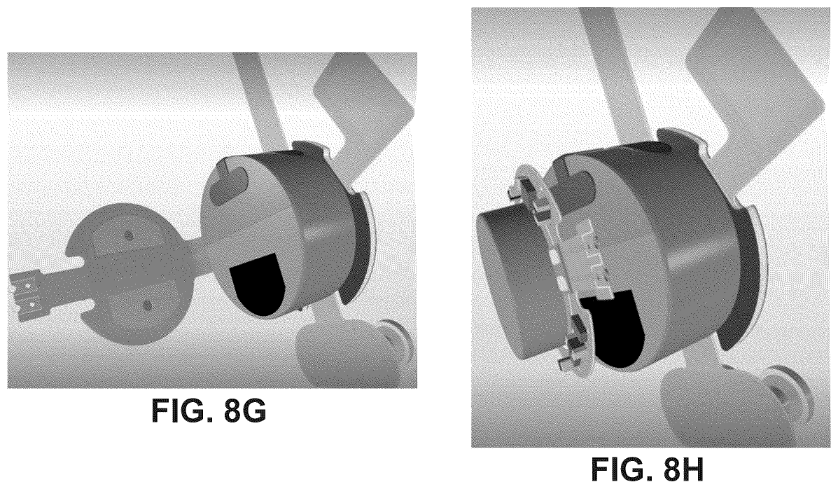

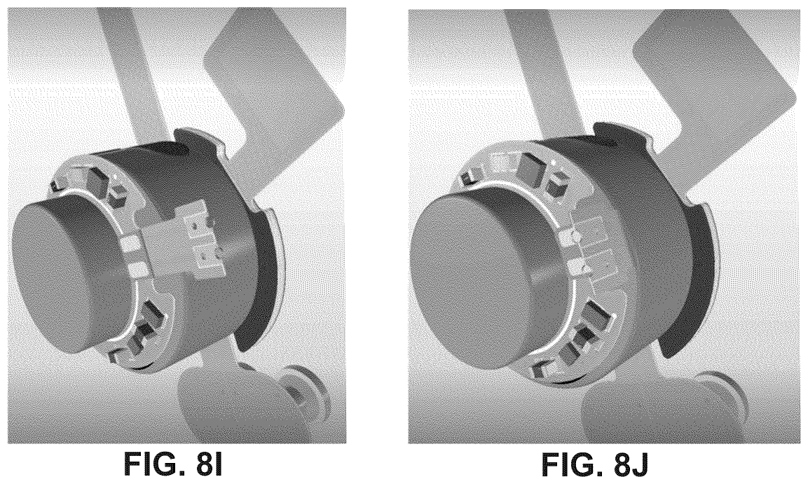

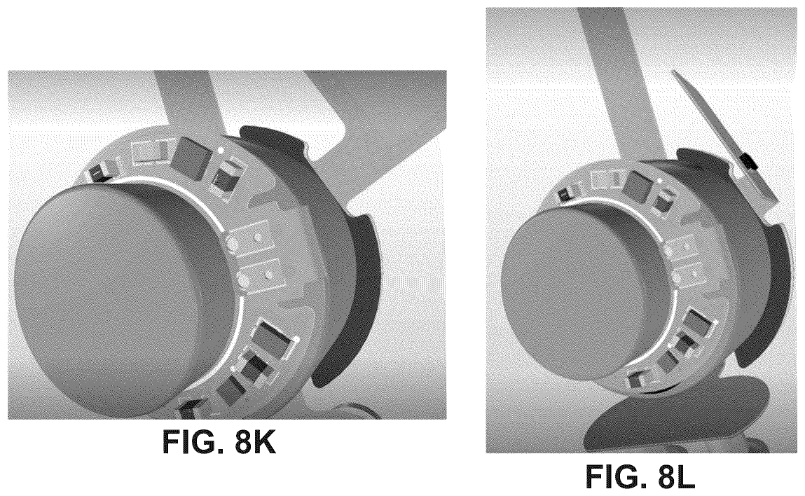

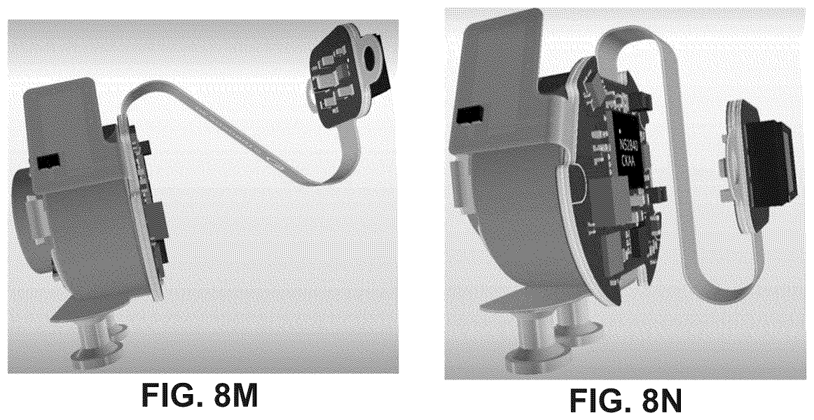

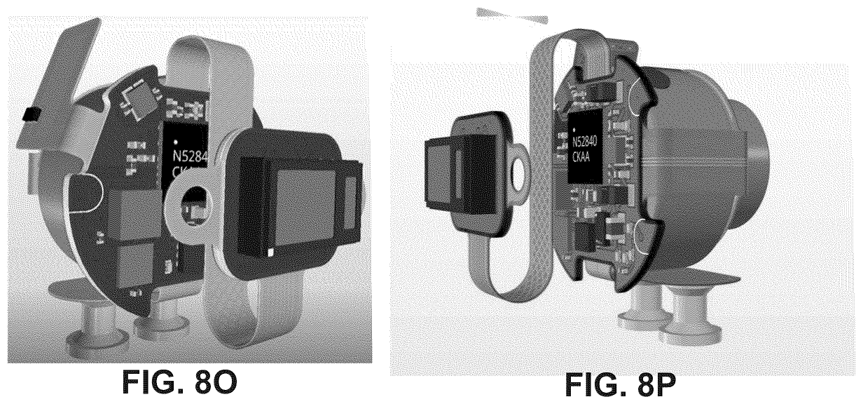

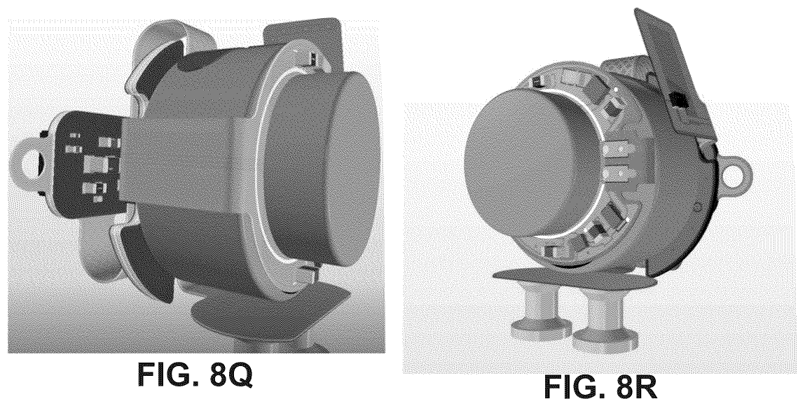

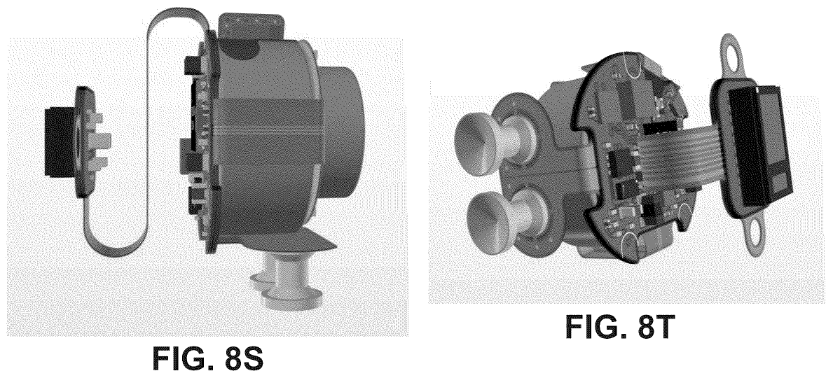

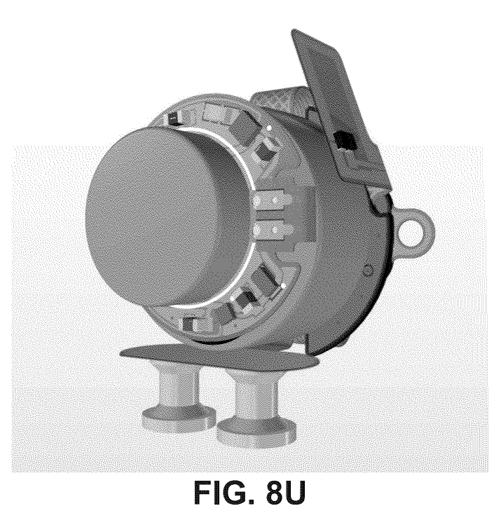

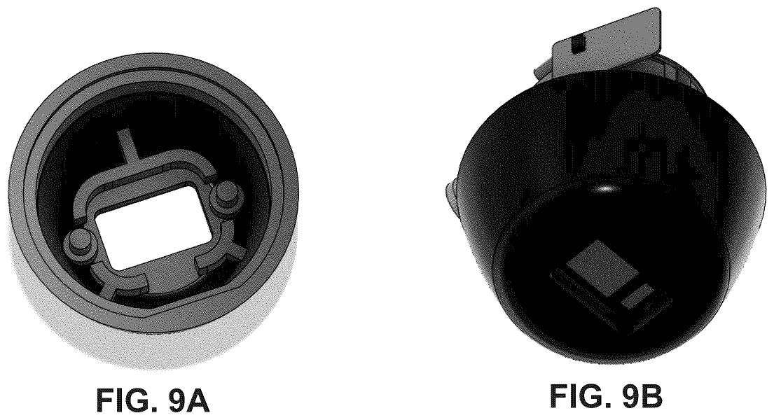

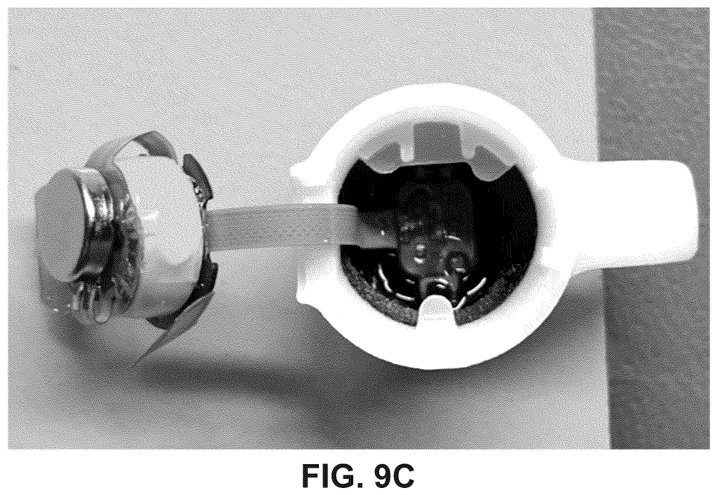

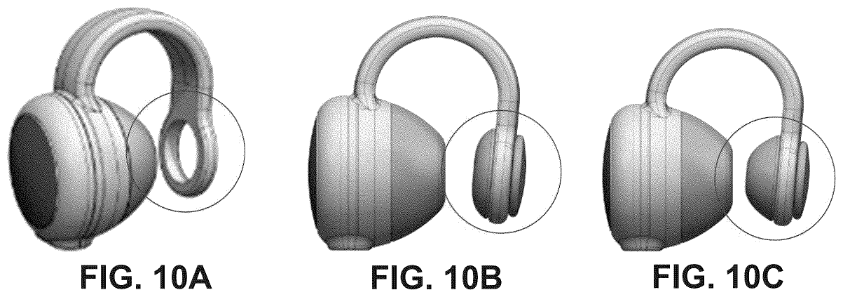

FIGS. 8A-8U are renderings of a flexible electronics assembly (e.g., electronics 760 of FIG. 7A-7C) for a wearable monitor, according to some embodiments. FIGS. 9A-9B are renderings of an earpad (e.g., an earpad of a first body portion) with optical sensor circuitry positioned within a recess of the earpad, according to some embodiments. FIG. 9C is a photographic image showing an underside view of an assembled earpad with optical sensor circuitry, according to an embodiment. FIGS. 10A-10C are drawings of a wearable monitor, showing a grommet without an earpad, with a thin earpad, and with a thick earpad, respectively, according to some embodiments.

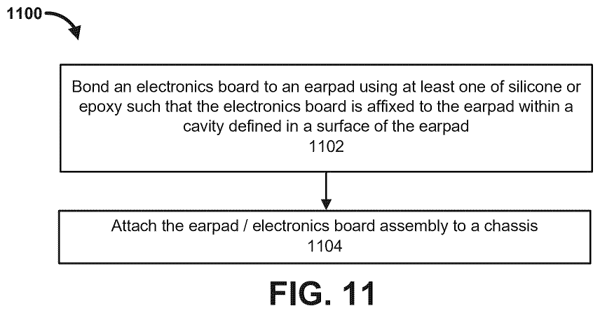

FIG. 11 is a flow diagram showing a method of manufacturing a wearable monitor, according to some embodiments. As shown in FIG. 11, the method 1100 includes at 1102 bonding an electronics board to an earpad (e.g., flexible earpad 724 of FIG. 7C) using at least one of silicone or epoxy such that the electronics board is affixed to the earpad within a cavity defined in a surface of the earpad, and at 1104 attaching the earpad to a chassis.

In some embodiments, an apparatus for monitoring an oxygen saturation level of a wearer of the apparatus includes a first body portion, a second body portion, and a connection member. The first body portion includes a processor, a memory operably coupled to the processor, at least one light-emitting diode, and at least one optical sensor configured to detect, during operation, a reflected portion of light emitted from the at least one light-emitting diode. The second body portion includes a grommet, and the connection member is mechanically coupled to each of the first body portion and the second body portion. The memory stores instructions to cause the processor to cause light to be emitted from the at least one light-emitting diode when the apparatus is positioned on an ear of a wearer and during operation of the apparatus, and to calculate a blood oxygen saturation level of the wearer based on the reflected portion of light.

In some implementations, the second body portion further includes a flexible earpad. Alternatively or in addition, the first body portion can include a flexible earpad. Alternatively or in addition, the apparatus further comprises a vibration motor, the memory further storing instructions to operate the vibration motor in response to detecting an event. The event can include at least one of: detecting an alarm condition, detecting a low battery condition, or detecting a successful positioning of the apparatus on the ear of the wearer.

In some embodiments, the connection member can include nylon. Alternatively or in addition, the first body portion can include a flexible earpad, and the at least one optical sensor can be positioned (e.g., potted) within a cavity defined in the flexible earpad. For example, the at least one optical sensor can be secured within the cavity by an epoxy.

The processor and the memory can be positioned on a circuit board, and the at least one optical sensor can be electrically coupled to the circuit board via a flexible conductor. The flexible conductor can be substantially S-shaped.

In some embodiments, the apparatus also includes at least one of a microphone or a speaker operatively coupled to the processor, and the memory further stores instructions to cause the processor to activate the at least one of the microphone or the speaker in response to detecting an alarm condition.

In some embodiments, the apparatus also includes a wireless transceiver, operatively coupled to the processor and configured to communicate with a mobile software application, the memory further storing instructions to cause the processor to send signals representing measurement data to the mobile software application via the wireless transceiver.

In some embodiments, the memory further stores instructions to cause the processor to send signals, according to a predetermined schedule, to a mobile software application, the signals representing measurement data.

In some embodiments, the apparatus also includes at least one sensor operatively coupled to the processor, the at least one sensor including at least one of: a body temperature sensor, an air quality sensor, a humidity sensor, an altimeter, or a barometric pressure sensor, the memory further storing instructions to cause the processor to store, in the memory, data collected by the at least one sensor.

In some embodiments, the apparatus also includes at least one sensor operatively coupled to the processor, the at least one sensor including at least one of: a body temperature sensor, an air quality sensor, a humidity sensor, an altimeter, or a barometric pressure sensor, the memory further storing instructions to cause the processor to send signals, according to a predetermined schedule, to a mobile software application, the signals representing data collected by the at least one sensor.

In some embodiments, the reflected portion of light is a first reflected portion of light and the instructions to cause the processor to calculate the blood oxygen saturation level of the wearer include instructions to cause the processor to calculate the blood oxygen saturation level of the wearer based on the first reflected portion of light and a second reflected portion of light.

In some embodiments, the apparatus also includes an alert mechanism (e.g., a button) operatively coupled to the processor, the memory further storing instructions to cause the processor to generate and send a signal representing an alert in response to detecting a user interaction with the alert mechanism. In some embodiments, the apparatus is waterproof. In some embodiments, the second body portion does not include any electronic components.

In some embodiments, a kit includes an ear-wearable apparatus, a charger case, and a plurality of flexible earpads. The ear-wearable apparatus is configured to be positioned on an ear of a wearer, and the apparatus includes a first body portion, a second body portion, and a connection member mechanically coupled to each of the first body portion and the second body portion. The first body portion has a processor, a memory operably coupled to the processor, at least one light-emitting diode, and at least one optical sensor configured to detect, during operation, a reflected portion of light emitted from the at least one light-emitting diode, the reflected portion of light being reflected from a surface of the ear of the wearer. The memory stores instructions to cause the processor to calculate a blood oxygen saturation level of the wearer based on the reflected portion of light. The charger case is configured to contain and supply power to the apparatus. The plurality of flexible earpads, each flexible earpad from the plurality of flexible earpads configured to be mechanically coupled to the second body portion. The second body portion can include an annular opening, and each flexible earpad from the plurality of flexible earpads can be configured to be mechanically coupled to the second body portion via the annular opening. Alternatively or in addition, the apparatus and the charger case can be configured such that data can be exchanged between the apparatus and the charger case (e.g., via a pair of electrical contacts).

Wearable monitors of the present disclosure, according to some embodiments, can include one or more (e.g., any combination) of the following capabilities:
Continuous oxygen monitoring;
Heart rate monitoring;
Generating and sending alerts;
Activation of a pre-defined emergency plan;
Interaction with a mobile software application; and/or
Predictive analytics (e.g., predictions of one or more biometric parameters, conditions, events, etc. as discussed herein).

In some embodiments, a wearable monitoring system includes a wearable monitor and a mobile software application (“mobile app”) running on a compute device of a user or wearer of the wearable monitor. During operation of the wearable monitor (i.e., when the wearable monitor is powered on and being worn by the wearer), the wearable monitor can continuously monitor the oxygen level of the wearer, for example by comparing a measured oxygen level to a pre-defined oxygen level threshold. Based on the monitoring, and in response to detecting that a measured oxygen level is less than the pre-defined oxygen level threshold, the wearable monitor can generate an alert, the alert including a representation of the low oxygen condition, and send a signal representing the alert to the mobile app to cause an alert (including one or more of text, graphics, video indications, and audio indications) to be displayed and/or played by the mobile app and the compute device (e.g., via a graphical user interface (GUI) of the compute device and/or one or more speakers thereof).

In some embodiments, during operation, the wearable monitor detects data associated with one or more physiological conditions of the wearer (collectively referred to herein as “vital signs”), including, but not limited to, oxygen level, heart rate, etc., for example at multiple instances over time. The wearable monitor can, in response to detecting the vital signs, store the vital signs in a memory of the wearable monitor. Alternatively or in combination, the wearable monitor can send a signal representing the vital signs to one or more remote compute devices (e.g., a mobile compute device of a user or the wearer, optionally running the mobile app) for storage and/or display (e.g., via a GUI). The vital signs can subsequently be accessible and retrievable by the wearer or user. In addition, the wearer can provide access to the vital signs to a medical practitioner, for example by providing the medical practitioner with access credentials (for the wearable monitor and/or the mobile app) and/or by generating and sending (via the mobile app) an email or other message, including a representation of the vital signs, to the medical practitioner.

In some embodiments, a wearable monitoring system includes a wearable monitor and a mobile app running on a compute device of a user or wearer of the wearable monitor. The wearable monitoring system is configured to generate and send one or more alerts when a vital sign is determined not to comply with a predetermined condition (optionally customizable by the wearer/user). For example, the wearable monitoring system (e.g., the wearable monitor and/or the mobile app) can be configured to generate and send one or more alerts when an oxygen level, measured by the wearable monitor, drops below a pre-defined (optionally customizable) level (also referred to herein as a “trigger” or “threshold value”). In addition, the wearable monitoring system can be configured to generate and send one or more alerts in response to when the user/wearer presses an alert mechanism (e.g., an actuatable button) on the wearable monitor, or otherwise interacts with the wearable monitor (examples of which include, but are not limited to, voice command, rotation of a component of the wearable monitor, sliding of a component of the wearable monitor, pressing or squeezing a component of the wearable monitor (e.g., for a predefined duration, with a predefined amount of force, a predetermined number of times in succession (e.g., 3 times), in a predefined pattern, etc.), removal of a component of the wearable monitor, removal of the entirety of the wearable monitor from the ear of the wearer, etc.).

The one or more alerts can include a representation of a “low oxygen” condition and/or an associated instruction to execute one or more commands, such as initiating a telephone call (e.g., via the mobile device running the mobile app) to, or otherwise contacting, emergency services (e.g., 911), sending an SMS, email or other message to one or more emergency contacts, emitting a sound from a sound emitter of the wearable monitor (e.g., an electronic beep sound effect emitted via a speaker), causing a vibration to be generated by a haptic feedback element of the wearable monitor (e.g., a piezoelectric transducer), transmitting (e.g., via a transceiver onboard the wearable monitor) a signal to a mobile device to cause an alert such as a sound effect and/or a vibration, etc. The user or wearer can, via the mobile app, define customized values for one or more of the following: threshold oxygen level(s), detection intervals (e.g., for oxygen levels, blood oxygen saturation levels, or other vital signs), frequency of calculation of vital signs (e.g., to calculate oxygen concentration at 1-30 second time intervals), threshold values for other vital signs and/or biometrics, emergency contact information (e.g., phone numbers, email addresses, names, etc.), emergency plan data, priority order of vital signs, priority order of emergency contacts, access permissions for healthcare providers, etc.

In some embodiments, the wearable monitoring system includes a predictive analytics capability and/or interacts with a predictive analytics system, to provide advance warnings (“pre-warnings”) to the wearer or user before an acute health episode occurs, for example by identifying/detecting patterns based on previous acute health episodes of the wearer/user. The predictive analytics capability can be implemented using software (e.g., artificial intelligence (AI), machine learning, or other algorithms) and/or hardware. The identifying/detecting the patterns can be performed by analyzing biometric data (including vital signs and/or other data detected and/or gathered by the wearable monitoring system), optionally in combination with geometric data and/or barometric data collected using the wearable monitoring system. For example, collected geometric data and/or barometric data can be compared with historical data pertaining to occurrences of low oxygen alerts/warnings and/or invocations of emergency services (e.g., 911 calls) to identify one or more patterns or correlations that can be used to predict a next alert for a wearer, to predict sets of conditions under which a wearer is likely to experience an alert event, to generate one or more advance warnings for display via a GUI of the wearer's mobile compute device, etc.

In some embodiments, the predictive analytics capability may be implemented using one or more machine learning models trained on historical biometric and/or environmental data. For example, a long short-term memory (LSTM) neural network may be employed to analyze time-series data such as SpO₂ variability, heart rate trends, altitude changes, and/or the like, to predict acute health events. In some embodiments, the predictive analytics capability leverages anonymized, crowd-sourced datasets to improve model accuracy across diverse populations. Regulatory compliance measures, such as adherence to HIPAA and GDPR standards, may be incorporated to ensure secure handling of sensitive health data. The predictive analytics capability may generate early-warning alerts displayed via a graphical user interface of a mobile application, enabling proactive intervention.

In some embodiments, a wearable monitoring system includes a wearable, hardware-based earpiece, and a mobile app that is compatible with Windows®, iOS®, and Android® compute devices. The earpiece is configured to communicate with a compute device running the mobile app, for example using a power-efficient, lightweight wireless protocol, such as Bluetooth®, BLER, ZigBee®, Z-Wave®, 6LoWPAN®, Thread®, WiFi-Ah® (HaLow®), 2G® (GSM), 3G®, 4GR, LTER Cat 0®, Cat 1®, Cat 3®, LTE-M1®, Narrowband IoT® (NB-IoT®), 5GR, NFC®, RFID, SigFox®, LoRaWAN®, Ingenu®, Weightless-N®, Weightless-P®, Weightless-WR, ANT®, ANT+R, DigiMesh®, MiWi®, EnOcean®, Dash7®, or WirelessHART®. The earpiece includes one or more of the following components: one or more light-emitting diodes (LEDs), one or more photosensors/photodetectors, one or more lightweight power-efficient wireless sensors, a speaker, a microphone, one or more air quality monitoring sensors, and one or more body temperature sensors. During operation of the earpiece, the one or more LEDs can transmit light, generated by the one or more LEDS, through a portion of an ear of a wearer of the earpiece, such that the transmitted light is detected by the one or more photosensors/photodetectors, the one or more photosensors/photodetectors disposed on an opposite side of the portion of the ear, as compared with the one or more LEDs. An amount of the light that is absorbed by the ear can be determined/calculated based on the amount of transmitted light detected by the one or more photosensors/photodetectors. A blood oxygen saturation (SpO2) level can then be calculated, based on the amount of light absorbed by the ear, for example using the Beer-Lambert Law (“Beer's Law”). The calculation of the amount of the light absorbed by the ear and/or the calculation of the SpO2 level can be performed by a processor of the earpiece, the mobile app, and/or via a processor of a remote compute device in communication with the earpiece.

In some embodiments, a wearable monitoring system includes a wearable, hardware-based earpiece (wearable monitor), a charger (e.g., a charging “pod,” as shown in FIGS. 1F-1J), and a mobile app. The wearable monitor can be in continuous communication via the mobile app, e.g., using one or more wireless antennas or sensors. The charger can be a wireless charger, configured to wirelessly charge the earpiece when the earpiece is at least partially physically received within the charger (and, optionally, when a cover or lid of the charger is closed). The earpiece can be configured to generate and/or send an alarm in response to detecting that an oxygen level of a wearer is lower than a pre-defined threshold value. In addition, the earpiece can be configured to generate and/or send multiple alarms, in response to multiple detections, over time, of an oxygen level of the wearer being lower than the pre-defined threshold value. A frequency of generating and/or sending the alarms can increase in response to detecting that differences between measured/detected/calculated oxygen levels of the wearer and the pre-defined threshold value are getting larger over time (i.e., the measured/detected/calculated oxygen levels of the wearer are decreasing over time, and are all lower than the pre-defined threshold value). Similarly, a frequency of generating and/or sending the alarms can decrease in response to detecting that differences between measured/detected/calculated oxygen levels of the wearer and the pre-defined threshold value are getting smaller over time (i.e., the measured/detected/calculated oxygen levels of the wearer are increasing over time, and are all lower than the pre-defined value). In addition, an intensity or severity of the alarms can increase over time, and/or a type of alarm generated/sent can change over time, in response to detecting that differences between measured/detected/calculated oxygen levels of the wearer and the pre-defined threshold value are getting larger over time. For example, an emitted sound (from the wearable monitor and/or from the compute device running the mobile app) can get louder, a rate of a flashing light (on the wearable monitor and/or of the compute device running the mobile app) can increase, a text description of the alarms (e.g., presented to a wearer/user via a GUI of the mobile device running the mobile app) can change from “low” to “moderate,” or from “moderate” to high,” etc. Similarly, an intensity of the alarms can decrease over time, and/or a type of alarm generated/sent can change over time, in response to detecting that differences between measured/detected/calculated oxygen levels of the wearer and the pre-defined threshold value are getting smaller over time. For example, an emitted sound (from the wearable monitor and/or from the compute device running the mobile app) can get quieter/softer, a rate of a flashing light (on the wearable monitor and/or of the compute device running the mobile app) can decrease, a text description of the alarms (e.g., presented to a wearer/user via a GUI of the mobile device running the mobile app) can change from “high” to “moderate,” or from “moderate” to low,” etc. The alarms can terminate when a most recently measured/detected/calculated oxygen level of the wearer reaches a “normal” level (e.g., at or above the pre-defined threshold value).

Embodiments set forth herein can be used to monitor symptoms of, predict the progression of, and/or as part of a treatment plan for one or more conditions such as pulmonary hypertension (PH), pulmonary arterial hypertension (PAH), idiopathic PAH (IPAH), pulmonary fibrosis, sceloderma, cystic fibrosis, lupus, sickle cell anemia, asthma, chronic obstructive pulmonary disease (COPD), heart disease, atrial fibrillation, acute mountain sickness (AMS), and Eisenmenger's Syndrome.

In some embodiments, a wearable monitor is configured to continuously or intermittently monitor vital signs such as oxygen levels and heart rates, and send data associated with the vital signs (e.g., via the mobile app) for storage in records of a memory or other storage repository, via a mobile app (e.g., implemented using a cloud-based server). In some such instances, when storing the vital signs, the mobile app can also cause the storage of some or all of the following additional information: GPS location of the wearer, altitude of the wearer (e.g., retrieved using a Google application programming interface (API)), an indication of room air quality (e.g., detected by an onboard sensor of the wearable monitor), environmental temperature, and environmental humidity level.

A wearer or user of the wearable monitor can subsequently retrieve/download the records (e.g., based on a specified date or date range), for example to show their physician for purposes of diagnosis and/or investigation of causes behind undesirable fluctuations. Alternatively or in addition, the records can automatically be downloaded, for example according to a pre-defined, customizable schedule (e.g., daily, weekly, monthly), and emailed to the wearer and/or other users, medical providers, etc.

In some embodiments, a wearable monitor is configured to initiate a telephone call to emergency services (e.g., 911) in response to a wearer or user pressing an alert mechanism (e.g., an actuatable button of), or otherwise interacting with an interface of, the wearable monitor. The button press (or other interaction) can also trigger the activation of an onboard speaker and microphone, to facilitate the telephone call, such that the wearer or user can speak into the microphone and hear the other party on the telephone call via the speaker. This allows the wearer/user to communicate the situation to emergency services and request appropriate help. Optionally, the button press (or other interaction) can also trigger (e.g., concurrently) the generation and wireless sending of an alert via SMS text message to one or more user-defined emergency contact phone numbers (e.g., as defined in the mobile app).

Alternatively or in addition, in some embodiments, a wearable monitor is configured to generate and send SMS text messages to one or multiple (e.g., 3) emergency contacts pre-defined by a wearer (e.g., as part of a pre-defined emergency plan), in response to the wearer or a user (e.g., a bystander) pressing a button of (or otherwise interacting with an interface of) the wearable monitor. The SMS text messages can include one or more of the following: an alert message, vital sign data of the wearer, a current GPS location of the wearer, and an indication as to whether emergency services (e.g., 911) have already been called.

In some embodiments, during operation, a wearable monitor is positioned on a portion of an ear of a wearer, and is in continuous communication (e.g., via one or more wireless antennas, such as Bluetooth®, 4G®, or 5G® antennas) with a mobile app that is concurrently running on a mobile compute device of the wearer. The wearable monitor continuously or intermittently over time, detects oxygen levels and heart rates of the wearer, and sends signals to cause display of the detected oxygen levels and heart rates of the wearer via a GUI of the mobile compute device of the wearer. The display of the detected oxygen levels and heart rates can be, for example, in the form of a graph, plot, or chart. The display can be dynamically updated, in real time or substantially in real time, in response to new measurements of oxygen levels and heart rates. As used herein, “substantially in real-time” can refer to an event that occurs immediately following a predicate event, adjusted for processing-related delays (e.g., computation delay, transmission delay, etc.). When triggered, an alert can be displayed within the GUI, together with or instead of the displayed data.

In some embodiments, the wearable monitor is an internet-of-things (IoT) device and includes an onboard Long-Term Evolution (LTE) module/chip, for 5G connectivity to other compute devices within the IoT.

In some embodiments, a first housing portion of a wearable monitor includes one or more air quality sensors, a speaker, an alert mechanism (e.g., an actuatable button), and a microphone. In some such embodiments, two or more of the microphone, the speaker and the one or more air quality sensors “share” (i.e., are open to external air via) a common opening in an outer shell/wall of the first housing portion, while a remainder of the outer shell/wall of the first housing portion, as well as an entirety of an outer shell/wall of the second housing portion are sealed and waterproof. The one or more air quality sensors can be configured to detect one or more of: biogenic volatile compounds (BVOC), temperature, humidity, carbon monoxide, carbon dioxide, sulfur dioxide, nitrous oxide, particulate matter, ozone and/or other gases. For example, in some embodiments, the one or more air quality sensors are configured to detect temperature, humidity, and one or more BVOCs, and to output a relative “score” of ambient air quality. Alternatively or in addition, the first housing portion can include a Bluetooth® 5.1 Direction Finding capability, for example to identify relative locations of multiple users (e.g., patients within a hospital). Batteries described herein can be rechargeable or non-rechargeable. Body temperature sensor(s) described herein can include one or more thermally conductive probes and/or one or more non-contact temperature sensors, such as thermopile infrared (IR) sensors.

In some embodiments, a first housing portion of a wearable monitor includes analog processing circuitry and/or one or more internal measurement sensors. The one or more internal measurement sensors can include one or more of, for example: an altimeter, gyroscope, accelerometer, GPS sensor, magnetometer, galvanic skin response (GSR) sensor, or a humidity sensor. The processor of the wearable monitor can be operably coupled to each of the analog processing circuitry and the one or more internal measurement sensors.

Alternatively or in addition, in some embodiments, a memory of a wearable monitor stores instructions to cause the processor to detect that an alert mechanism of the wearable monitor has been interacted with (e.g., pressed) by a wearer (i.e., a manual alert), and, in response to detecting that the alert mechanism has been interacted with, generate and send (via the wireless transceiver) a message to one or multiple emergency contacts stored in the memory (e.g., as part of an emergency plan stored therein). The memory can also store instructions to cause the processor, in response to detecting that the alert mechanism has been interacted with, to: initiate a telephone call to emergency services (911), activate a speaker, activate a microphone, emit a sound to indicate an alarm, emit a light to indicate an alarm, generate and send (via the wireless transceiver) an alert message to the mobile app for presentation to a user via a GUI of the user's compute device, and cause storage to memory of an alert record including a date stamp, a time stamp, and measurement data collected from components of the wearable monitor (e.g., the air quality sensor, photodetectors, the body temperature sensor(s), the internal measurement sensor(s), etc.) at the time of the alert.

Alternatively or in addition, the memory can store instructions to cause the processor to compare a predetermined threshold stored in the memory with one or more measurements collected by one or more components of the wearable monitor. When the processor determines that the one or more measurements are undesirably below or undesirably above the predetermined threshold, the processor can detect that an alarm condition is present. The memory can also store instructions to cause the processor, in response to detecting the alarm condition, generate and send (via the wireless transceiver) a message to one or multiple emergency contacts stored in the memory (e.g., as part of an emergency plan stored therein). The memory can also store instructions to cause the processor, in response to detecting the alarm condition, to: initiate a telephone call to emergency services (911), activate the speaker, activate the microphone, emit a sound to indicate an alarm, emit a light to indicate an alarm, generate and send (via the wireless transceiver) an alert message to the mobile app for presentation to a user via a GUI of the user's compute device, and cause storage to memory of an alert record including a date stamp, a time stamp, and measurement data collected from components of the wearable monitor (e.g., the air quality sensor, the photodetectors, the body temperature sensor(s), the internal measurement sensor(s)) at the time of the alert.

In some embodiments, to commence use of a wearable monitor, a wearer positions the wearable monitor about a portion of the wearer's ear (e.g., the upper ear, such as the helix, scapha, or pinna of the ear), in a wear configuration. In a first example wear configuration, the first housing portion of the wearable monitor is in contact with or adjacent to an anterior or front surface of the ear, and the second housing portion of the wearable monitor is in contact with or adjacent to a posterior or back/rear surface of the ear. In a second example wear configuration, the second housing portion of the wearable monitor is in contact with or adjacent to an anterior or front surface of the ear, and the first housing portion of the wearable monitor is in contact with or adjacent to a posterior or back/rear surface of the ear. Stated another way, when the wearable monitor is worn, the first housing portion and the second housing portion are positioned on opposite sides of the wearer's ear.

In some embodiments, a first light-emitting diode of a wearable monitor can be configured to emit light having a first wavelength, and a second light-emitting diode of the wearable monitor can be configured to emit light having a second wavelength different from the first wavelength.

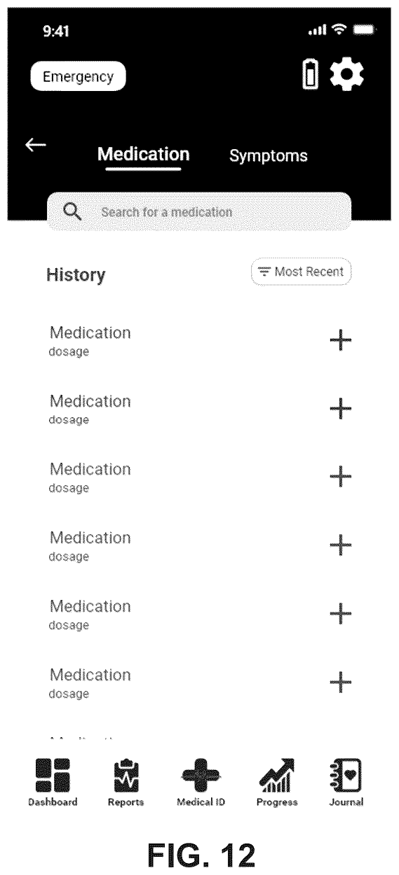

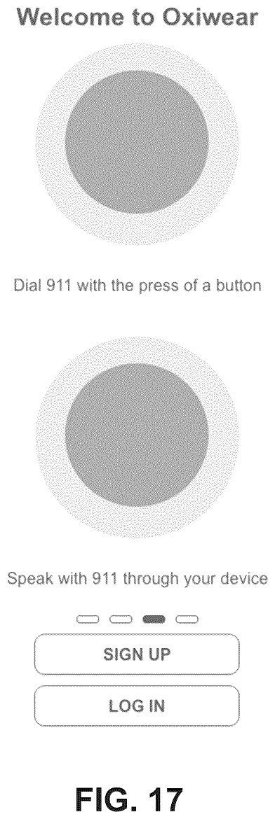

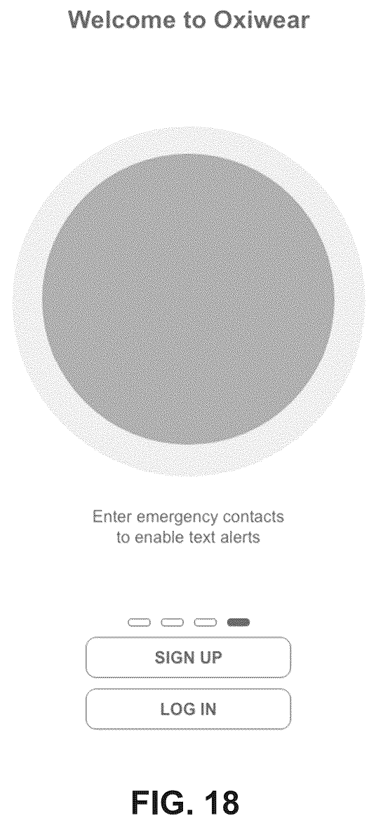

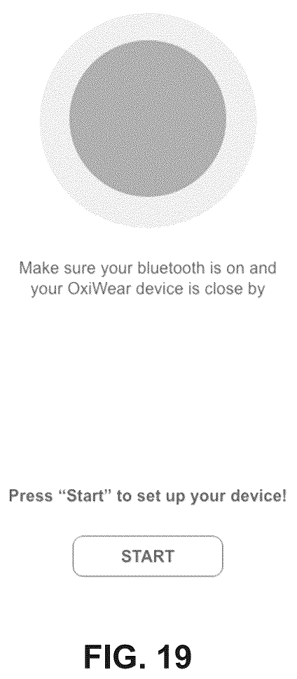

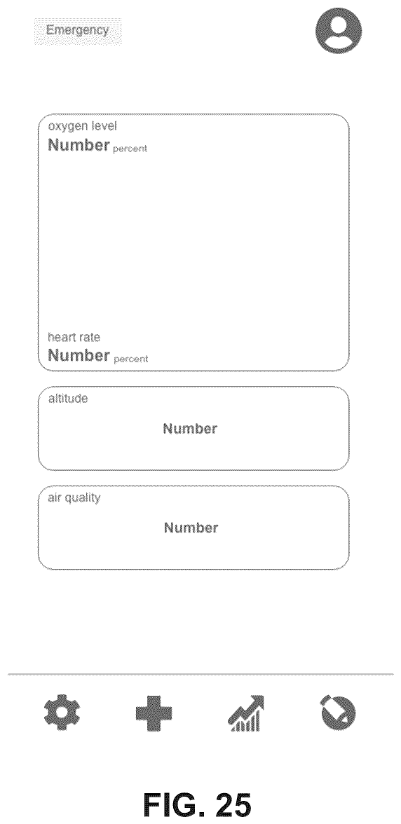

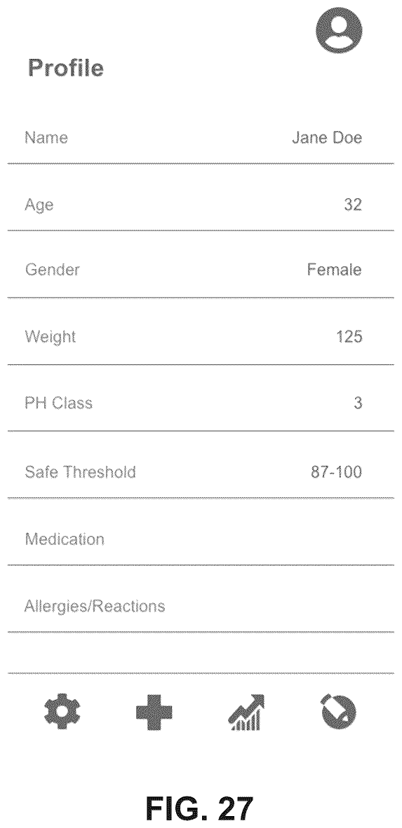

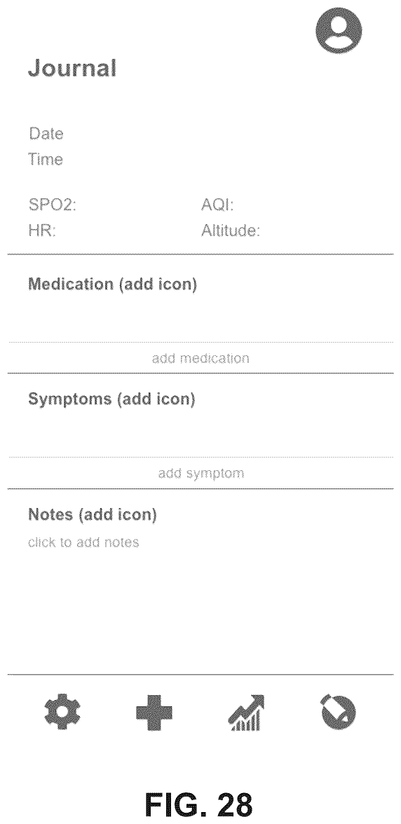

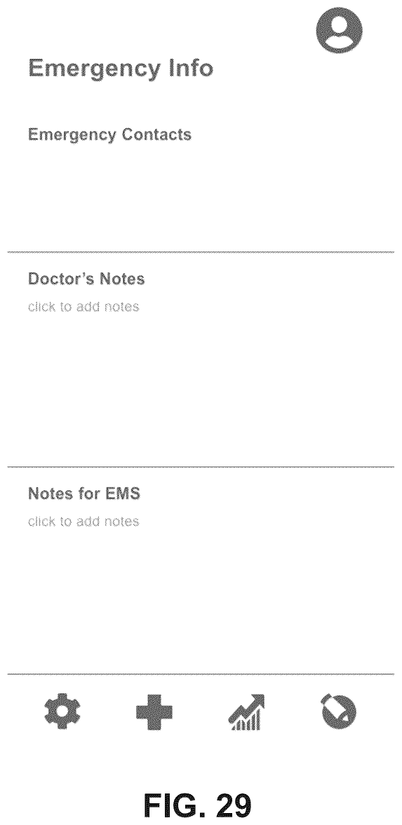

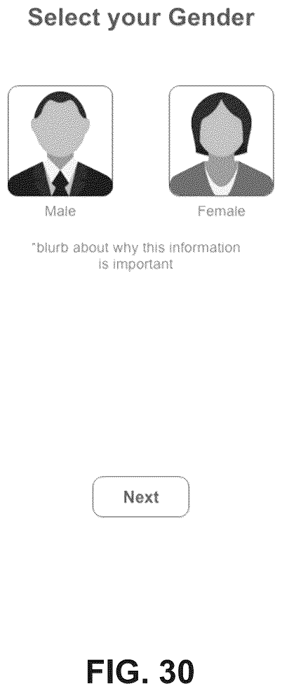

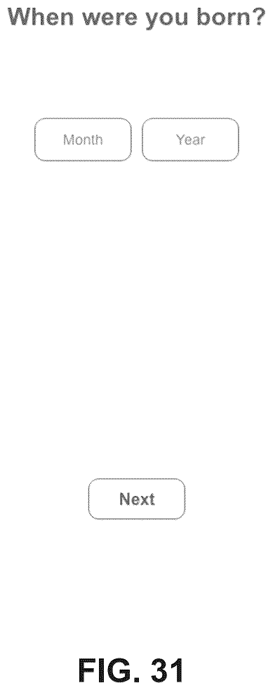

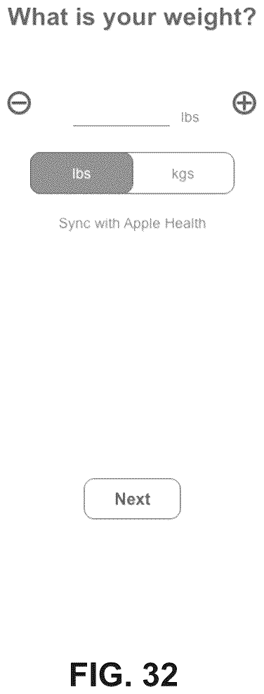

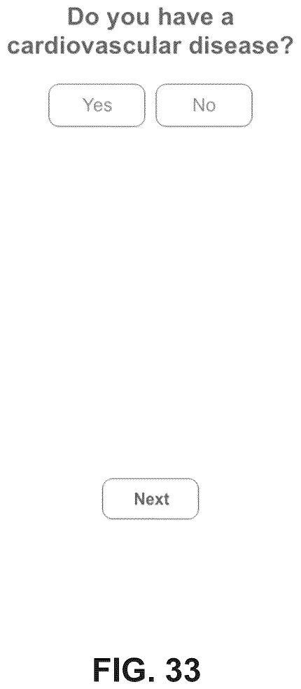

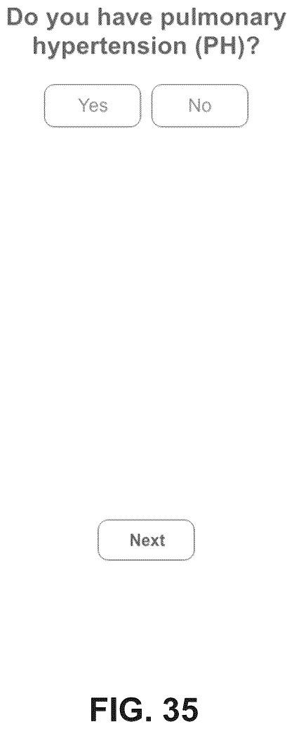

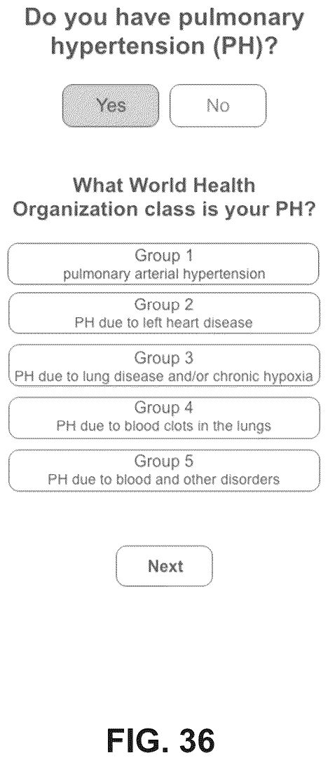

FIGS. 12-48 are wireframes of user interface screens (for GUI display) of a mobile app (e.g., running on a mobile compute device) that interacts with a wearable monitor (e.g., with any wearable monitor described herein), according to some embodiments. As shown in FIG. 12, a user of the mobile app (optionally also a wearer of an associated wearable monitor, as shown and described herein) can create a user profile in the mobile app. The creation of a user profile can include specifying one or more medications that the user is currently taking or has taken in the past. The user interface can include a search bar in which the user can enter search criteria pertaining to one or more medications, and by pressing ENTER, can cause a search to be executed for matches to the search criteria. The results identified via the search can then be displayed for selection via the user interface. The user can “add” medications to their user profile by clicking on the “+” symbol next to the relevant medication(s). Also shown in FIG. 12 are the available/navigable screens: “Dashboard,” “Reports,” “Medical ID,” “Progress” and “Journal.”

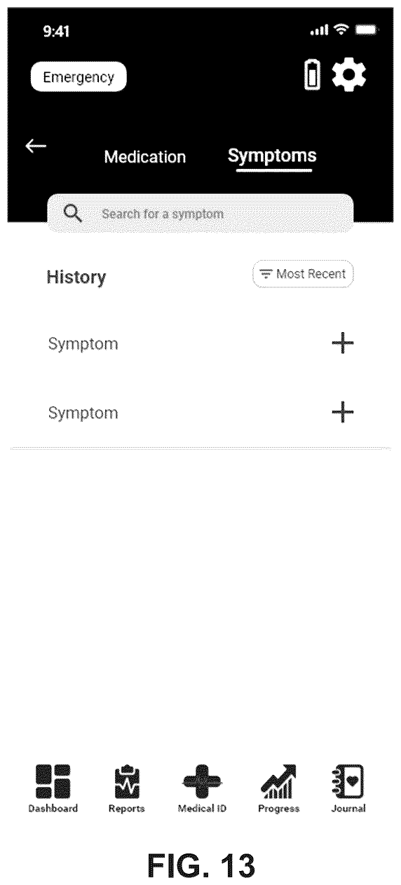

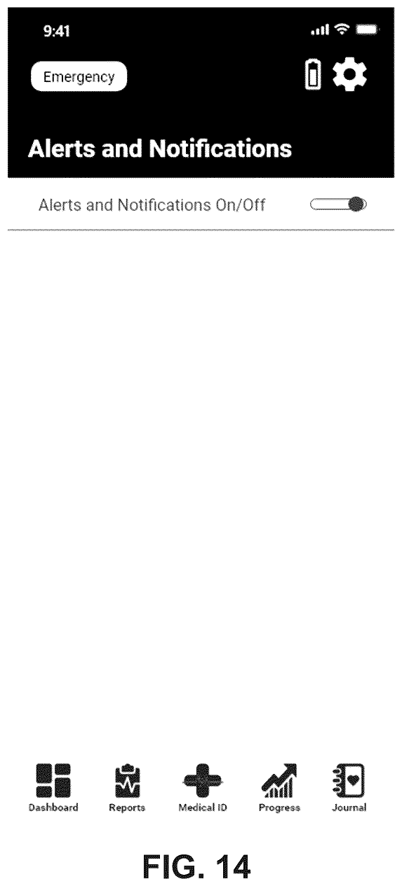

FIG. 13 shows that the creation of a user profile can also include specifying one or more symptoms that the user is currently experiencing or has experienced in the past. The use interface can include a search bar in which the user can enter search criteria pertaining to one or more symptoms, and by pressing ENTER, can cause a search to be executed for matches to the search criteria. The results identified via the search can then be displayed for selection via the user interface. The user can “add” symptoms to their user profile by clicking on the “+” symbol next to the relevant symptom(s). The user interface also includes an “Emergency” button that the user can select to trigger one or more alerts to be generated and/or sent. For example, selecting the “Emergency” button, similar to pressing the alert mechanism of the wearable monitor described herein, can trigger one or more of the following: generating and sending a message to one or multiple emergency contacts (e.g., stored as part of an emergency plan), initiating a telephone call to emergency services (911), activating speakerphone on the mobile compute device, emitting a sound to indicate an alarm, emitting a light (e.g., of a predetermined color and/or in a predetermined pattern) to indicate an alarm, displaying an alert message via the user interface, and causing storage, in memory, of an alert record including a date stamp, a time stamp, and measurement data collected from components of the wearable monitor. FIG. 14 shows an Alerts and Notifications user interface, via which the user can turn alerts and notifications on or off, using a slider.

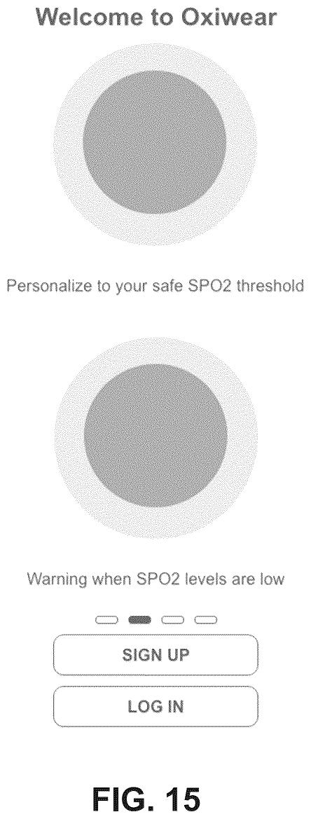

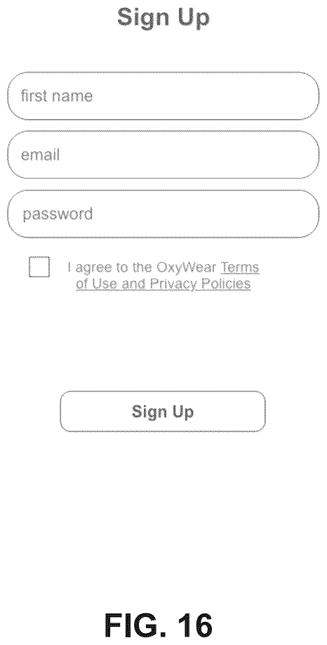

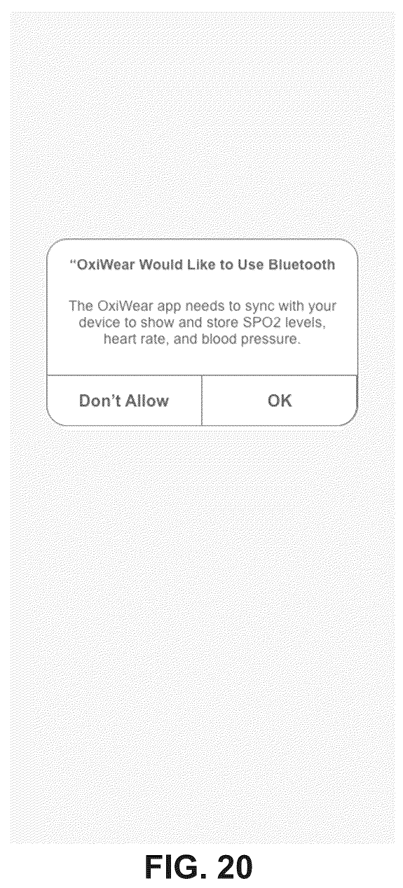

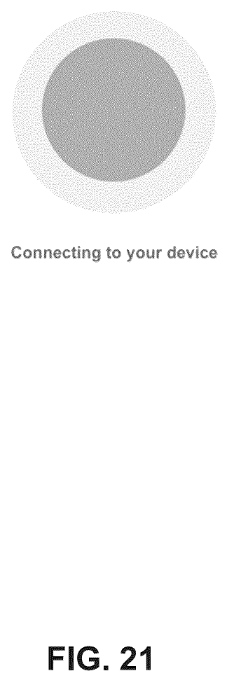

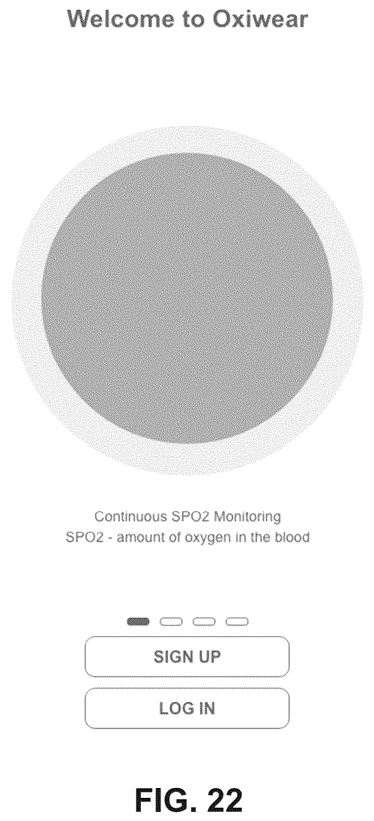

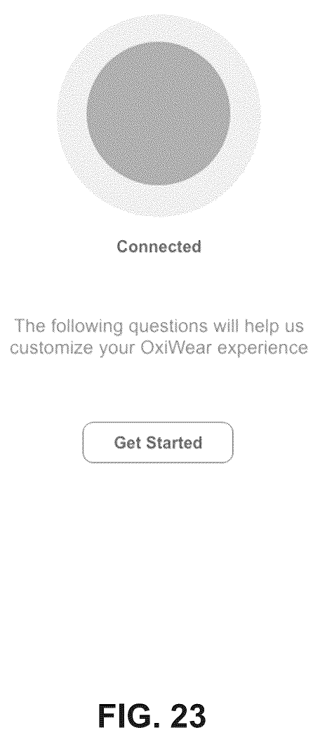

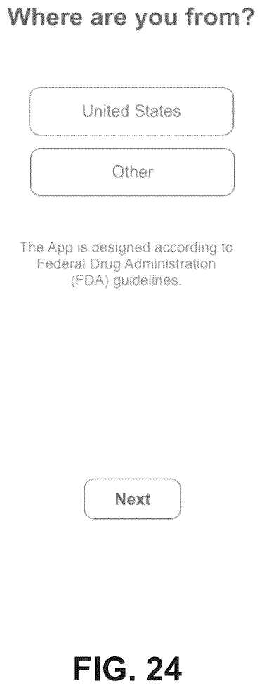

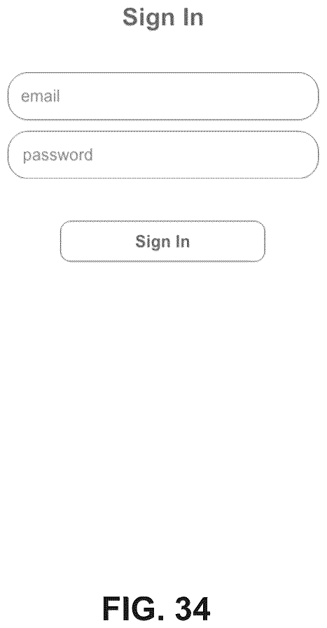

FIGS. 15-19 show welcome screens describing the mobile app's functionality, and via which a user can sign up for services (see FIG. 16), log in, set up, and personalize the mobile app. The mobile app can be personalized, for example, to include a representation of one or more safe blood oxygen saturation (SpO2) thresholds, such that a warning or alert is generated when the wearer's SpO2 is detected, by the wearable monitor, as being too low. FIG. 20 shows a synchronization request, in which the mobile app requests the user's input (“OK”) to authorize the mobile app to establish a Bluetooth® connection and synchronize (“sync”) the mobile app, via the Bluetooth® connection, with the wearable monitor. The user interface appearance, during synchronization, is shown in FIG. 21. FIG. 22 shows the login screen, and FIG. 23 shows an option for the user to “Get Started,” for example after a first login event by the user. FIGS. 24-36 show user interfaces of a questionnaire requesting input from the user as to: whether they are from the U.S., what the emergency threshold values for oxygen level, heart rate, altitude and air quality should be, settings (alerts and notifications, location services, unit preferences, language preference, app sync, and other personalization), profile data (name, age, gender, weight, pulmonary hypertension (PH) class (e.g., pulmonary arterial hypertension (PAH), pulmonary hypertension owing to left heart disease, chronic obstructive pulmonary disease, chronic thromboembolic pulmonary hypertension (CTEPH), or pulmonary hypertension with unclear multifactorial mechanisms), safe threshold, medication(s), and allergies/reactions), journal personalization, emergency information (emergency contacts, doctor's notes, notes for EMS), gender, birth month and year, weight, whether or not they have a cardiovascular disease, and whether or not they have pulmonary hypertension (and, if so, what World Health Organization (WHO) classification). FIG. 34 shows a login screen, which may be completed by a user prior to accessing any of FIGS. 24-33 and FIGS. 35-36, or after setting up a user account or completing the “sign up” process referenced in FIG. 22 (e.g., by answering questionnaire questions shown in FIGS. 24-33 and FIGS. 35-36).

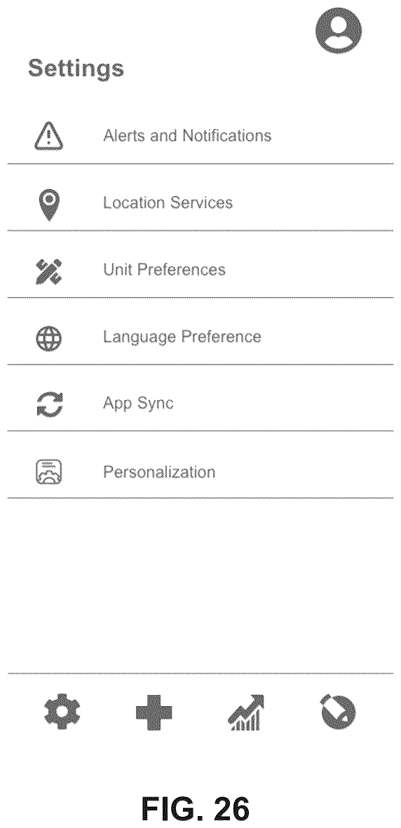

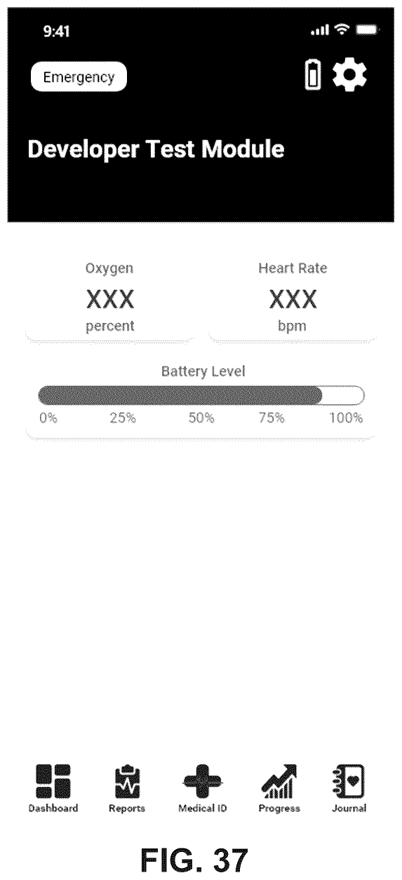

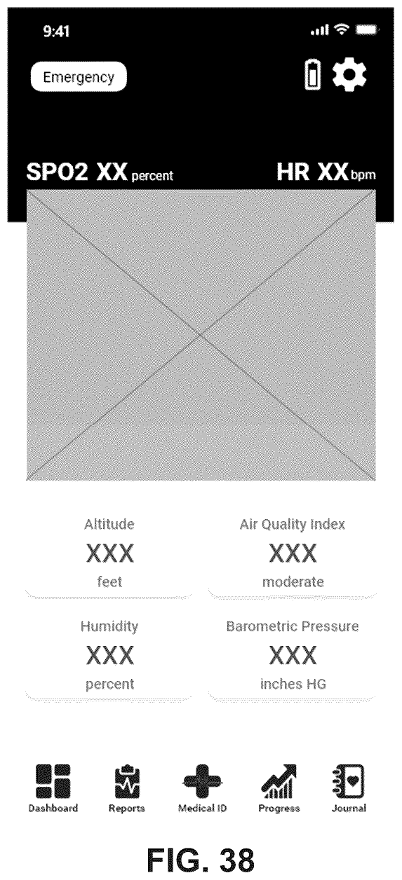

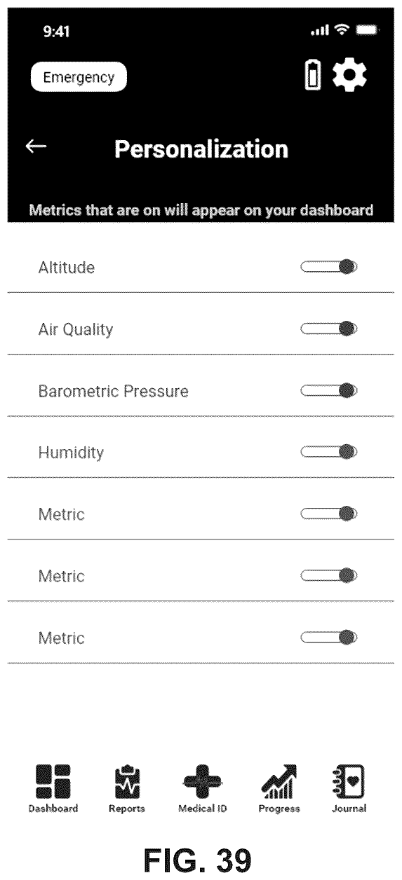

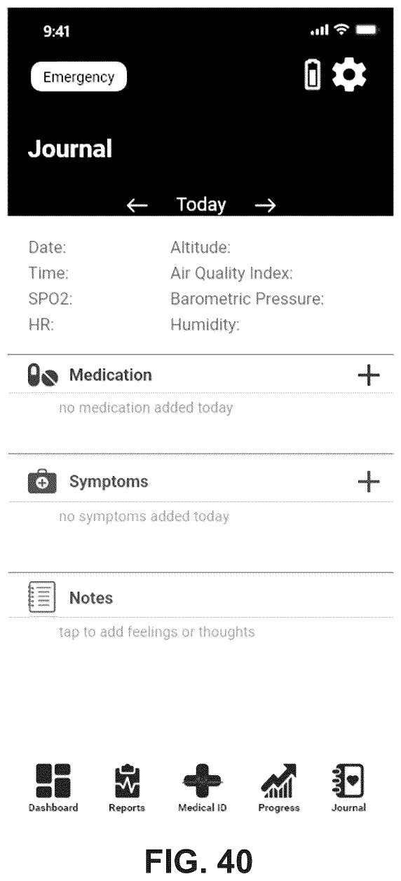

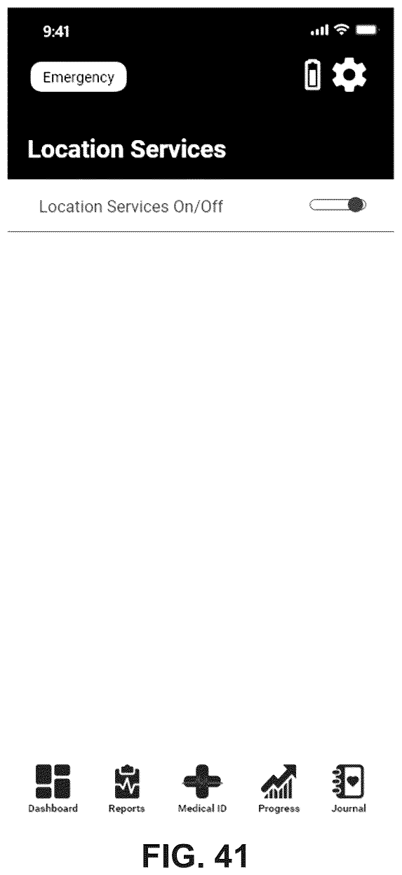

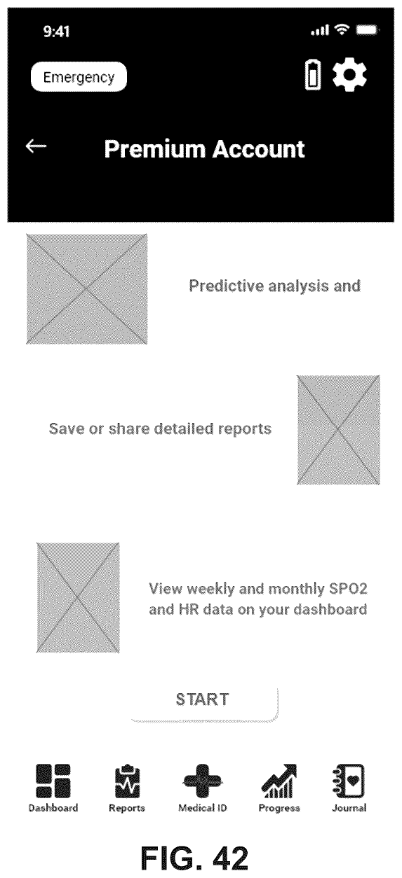

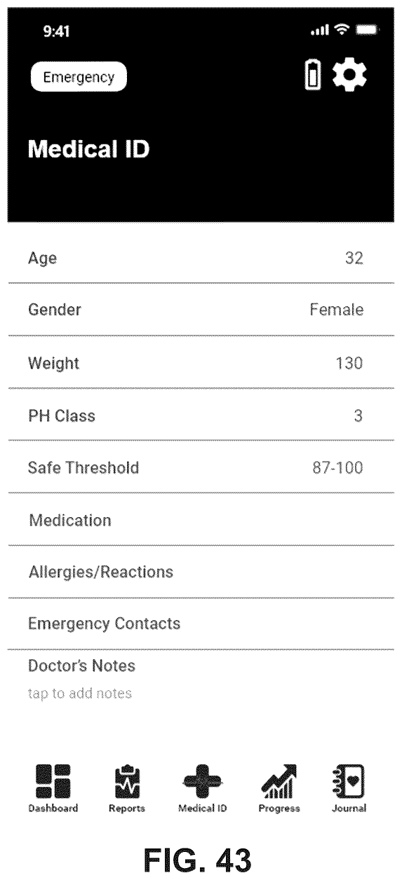

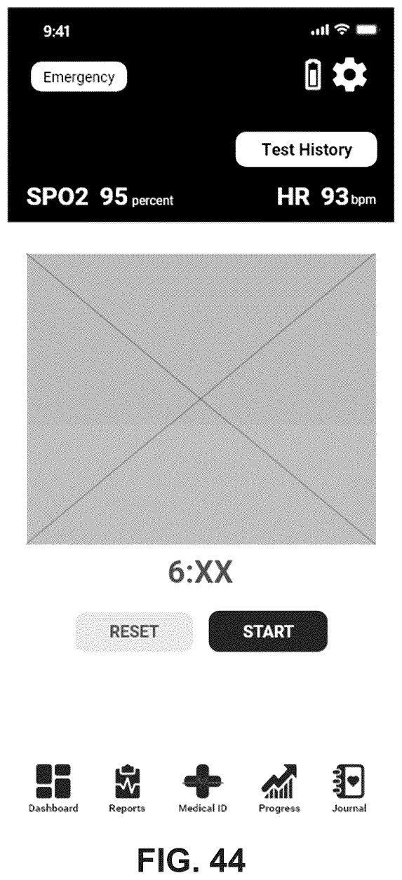

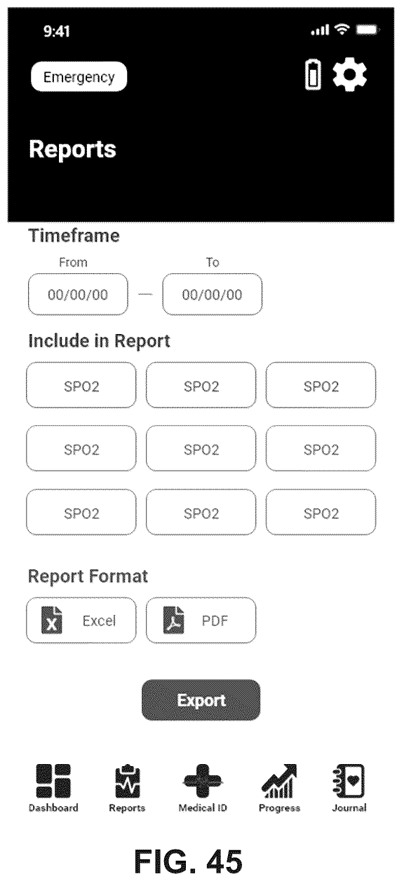

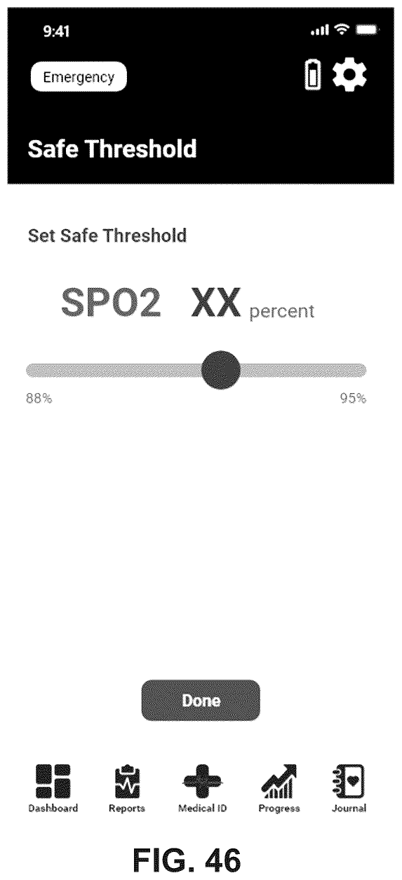

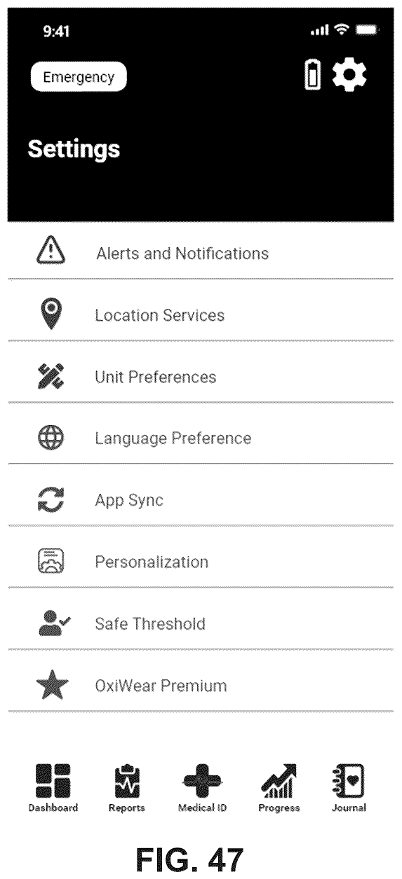

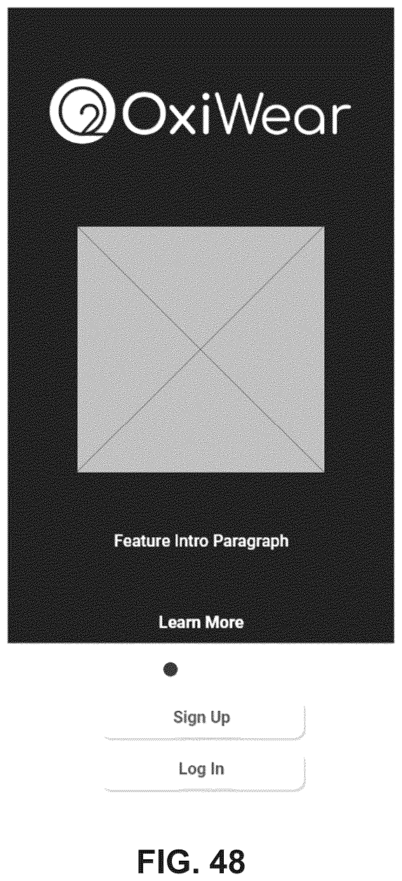

FIG. 37 shows a developer test module screen with a battery level indication and current oxygen level (percentage) and heart rate (beats per minute, BPM) readings. FIG. 38 shows current blood oxygen saturation level (percentage) and heart rate (beats per minute, BPM) readings, along with current altitude (feet), humidity (percent), air quality index (e.g., good, moderate, poor), and barometric pressure (inches mercury (Hg)). FIG. 39 shows a user interface via which the user can select which metrics they wish to have appear on their dashboard page. FIG. 40 shows an example journal entry for the current day, with date, time, SpO2, heart rate, altitude, air quality index, barometric pressure, and humidity values (e.g., average values, high values, etc.), together with medications taken on the current day (if any), symptoms experienced on the current day (if any), and notes entered by the user (if any). FIG. 41 shows a Location Services user interface, via which the user can turn location services (e.g., GPS location) on or off, using a slider. FIG. 42 shows a user interface accessible, for example, to users having a premium account, or providing the user with option to sign up for a premium account. The premium account can provide the user access to features such as predictive analysis, detailed report generation, and the presentation of weekly and monthly vital signs via the mobile app dashboard. FIG. 43 is a user interface showing a populated Medical ID tab. FIG. 44 is a user interface showing current oxygen level (percentage) and heart rate (BPM) readings, along with a resettable timer. FIG. 45 is a user interface showing user-selectable options for report generation, including timeframe, vital signs of interest, and report format (e.g., Microsoft® Excel® or Adobe® PDF®). FIG. 46 shows a Safe Threshold user interface, via which the user can set a threshold percentage SpO2 value, using a slider, and select “Done” when complete. FIG. 47 is a user interface showing Settings after Safe Thresholds have been set and a premium account has been established (as contrasted with the Settings shown in FIG. 26, which do not include a Safe Threshold or OxiWear Premium line item). FIG. 48 is a user interface showing an example landing screen that may display when the mobile app is first launched/opened (i.e., the first screen that a first-time user will see, and via which he/she can register for an account).

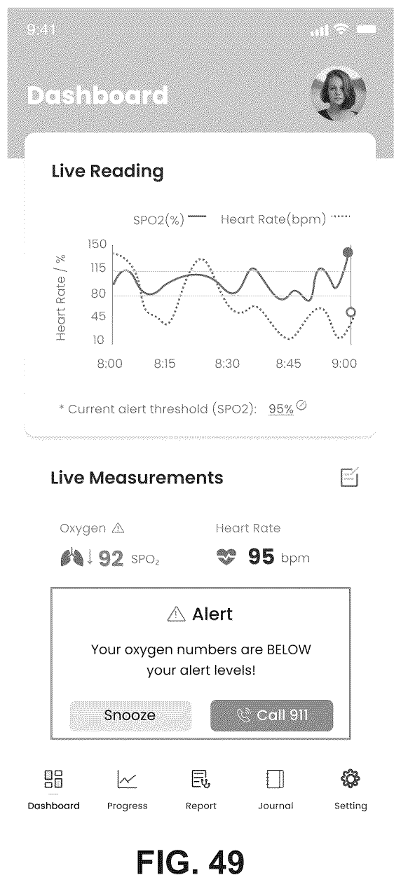

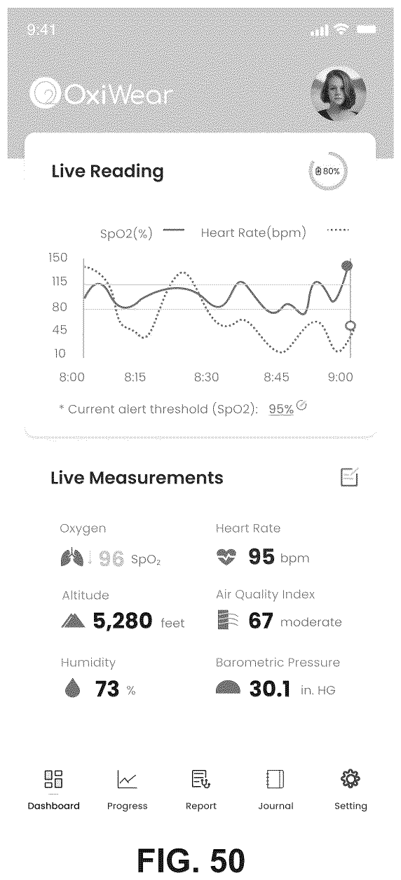

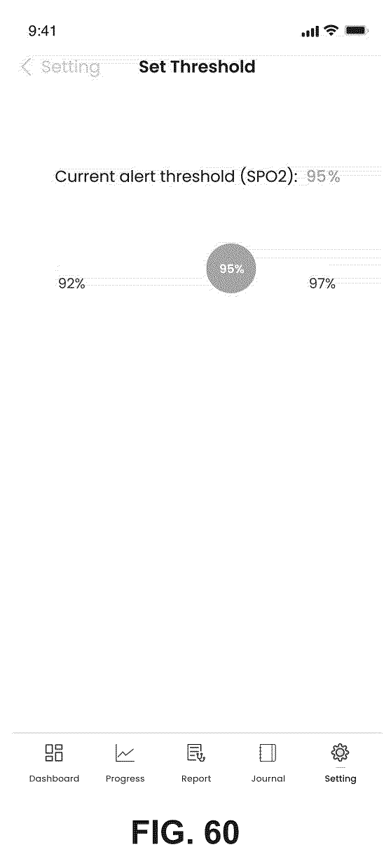

FIG. 49 shows a user interface including an alert sub-window disposed over a dashboard window, the alert sub-window being displayed in response to one or more SpO2 indicators being below a pre-determined threshold. In at least some embodiments, said pre-determined threshold is specified by a user as shown in FIG. 60. The alert sub-window comprises a user interface comprising one or more buttons that a user can interact with. For example, a user can interact with a snooze button to suppress the alert sub-window (e.g., for a predefined period of time, such as 5 minutes, 10 minutes, 30 minutes, etc.). The user can further interact with a “call 911” button configured to initiate a telephone call to an emergency dispatch office. FIG. 50 shows a dashboard depicting a plurality of measurements including biometric measurements for a user as well as factors that relate to or contribute to a user's oxygen level. For example, said plurality of measurements can include one or more live biometric readings for SpO2 blood oxygen level and/or heart rate. Said plurality of measurements can also comprise one or more environmental measurements for altitude, air quality index, humidity, and or barometric pressure. The dashboard can also depict a record of one or more previous measurements in graphical form. For example, a plurality of SpO2 and heart rate measurements sampled within the previous hour (or any other desired preceding time period) can be plotted on a graph, and said graph can be updated, in real time or substantially in real time, in response to new measurements of oxygen levels and heart rates.

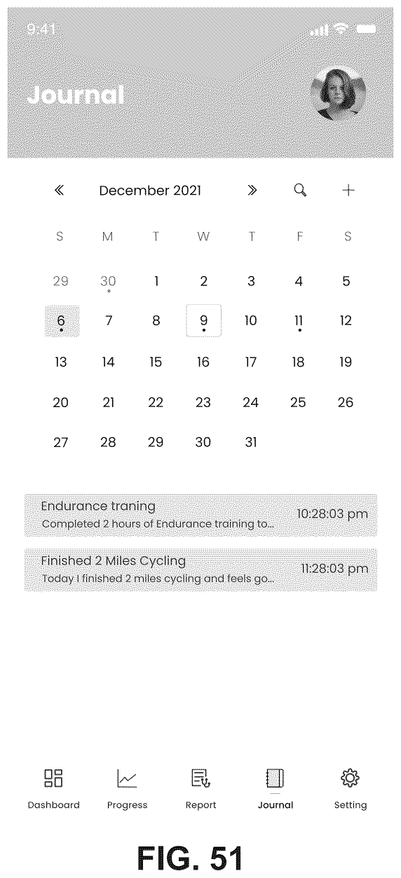

FIG. 51 shows a user interface comprising a monthly/calendar view of a user's journal entries. A user can enter a journal entry for a particular day using, for example, the interface shown in FIG. 40. Said journal entry can comprise date, time, SpO2, heart rate, altitude, air quality index, barometric pressure, and humidity values (e.g., average values, high values, etc.), together with medications taken on the current day (if any), symptoms experienced on the current day (if any), exercise and/or physical activity completed (if any), and notes entered by the user (if any). A user can interact with a calendar for a given month to view any journal entries entered for a day in the given month. A user can further use the calendar to select a day to enter a new journal entry for by interacting with a “+” icon or a similar interface/GUI element. A user can further use the calendar to search for a previous journal entry (e.g., using a keyword search, by interacting with the magnifying glass icon or similar interface/GUI element).

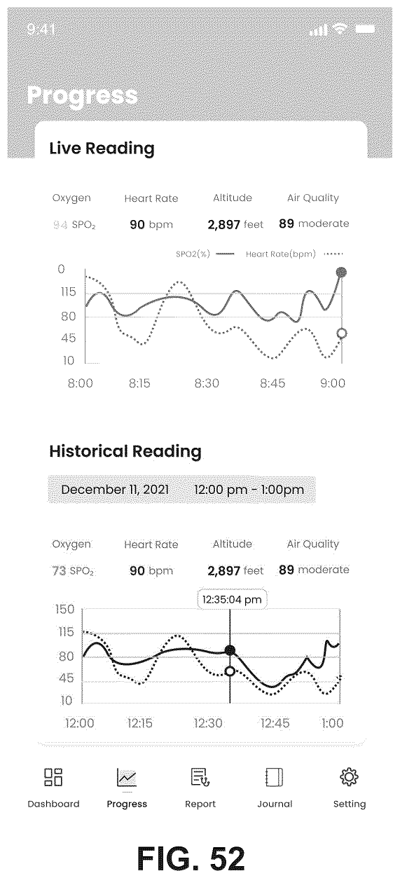

FIG. 52 shows a user interface comprising views of detected oxygen levels and heart rates. Said views can be, for example, in the form of a graph, plot, or chart. One view can depict a live reading that can be dynamically updated, in real time or substantially in real time, in response to new measurements of oxygen levels and heart rates. A second view can depict a plurality of detected oxygen levels, heart rates, and/or other measurements measured within a period defined by the user through a GUI. Said period can comprise a past time period and/or a past date/range (i.e., historical data/readings) for which the measurements were recorded.

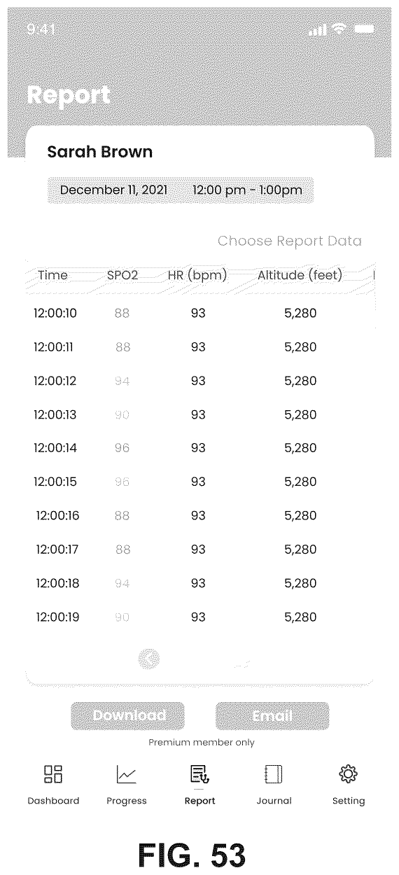

FIG. 53 shows a user interface for viewing and configuring a report. Said report can comprise measurements collected over a period defined by a user. Said report can comprise at least one of a plurality of vital/biometric measurements and/or a plurality of environmental measurements. For example, said report can comprise a plurality of SpO2, heart rate, and altitude measurements recorded or otherwise captured within a given period. Said period can comprise a past date (or date range) and/or time (or time range) defined by a user through a GUI. A user can download the report (e.g., as a file for viewing and/or local storage) and/or can generate and send an email containing the report via the user interface.

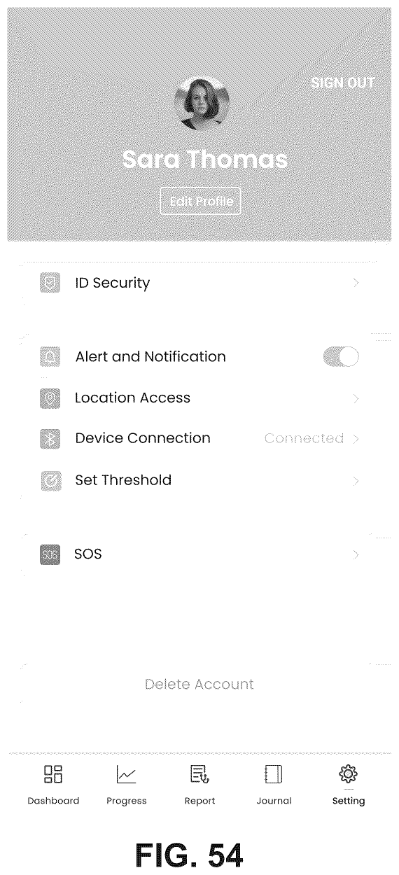

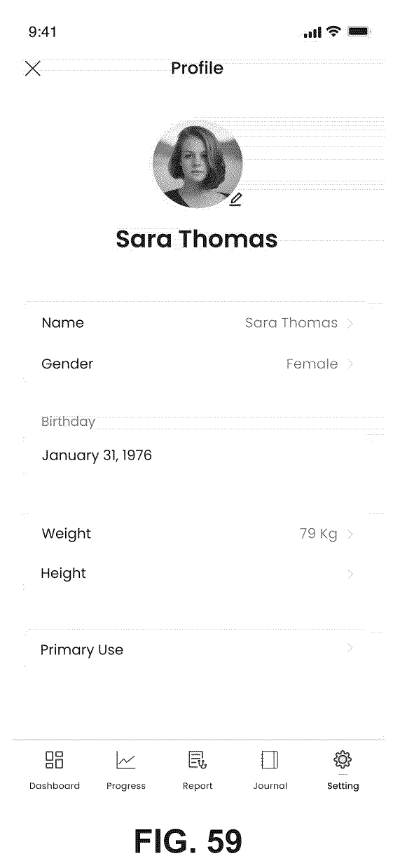

FIG. 54 shows a user interface through which a user can access a plurality of settings. The settings can include, for example, ID Security settings, an alert and notification toggle, location access settings, Bluetooth® and similar device connection settings related to a wireless communications protocol, threshold settings, and/or SOS/emergency notification settings. The user interface can be configured to provide users access to a GUI to edit the user's profile, as shown in FIG. 59. The user interface can also provide a button that a user can use to delete the user's account.

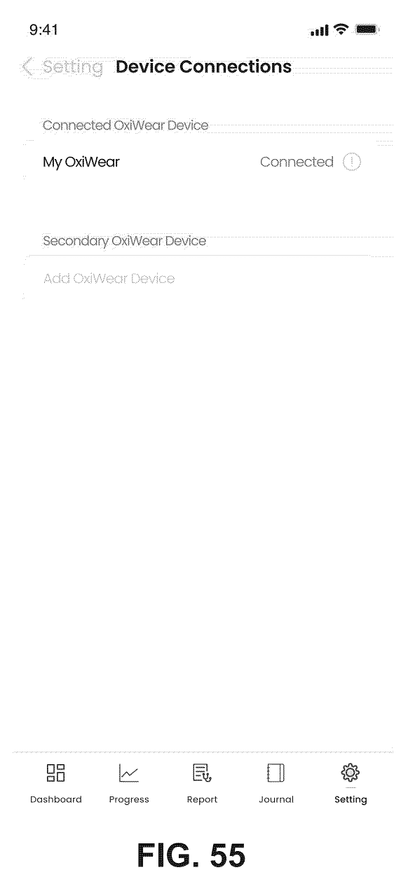

FIG. 55 shows a user interface through which a user can configure one or more connections between two or more devices. Said devices can comprise, for example, one or more wearable devices and a mobile device (e.g., a smartphone, tablet, laptop, smartwatch, or similar compute device). The mobile device can be the device on which the user interface of FIG. 55 is displayed. Said one or more connections can comprise, for example, one or more network connections using one or more wireless communications protocols (e.g., Bluetooth®, 4G®, 5GR, etc.). A user can access said user interface by interacting, for example, with the interface shown in FIG. 54.

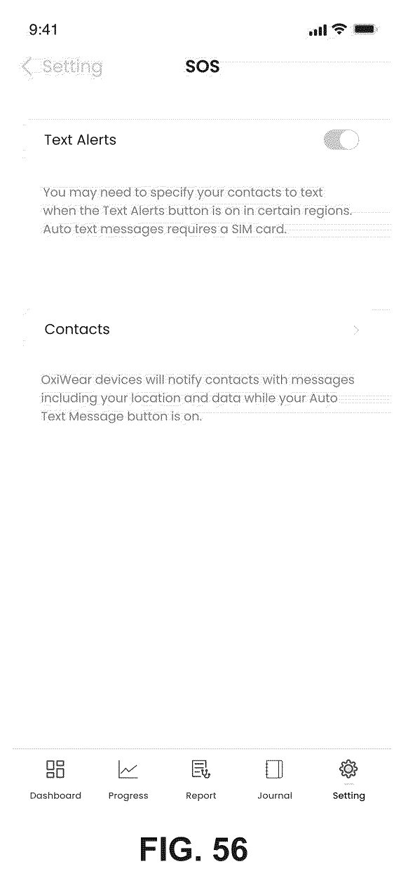

FIG. 56 shows a user interface for configuring SOS/emergency notification alerts. Said SOS alerts can comprise text alerts sent to one or more contacts defined by a user using said user interface. Said text alerts can comprise SMS text messages. A user can access said user interface using, for example, the interface shown in FIG. 54.

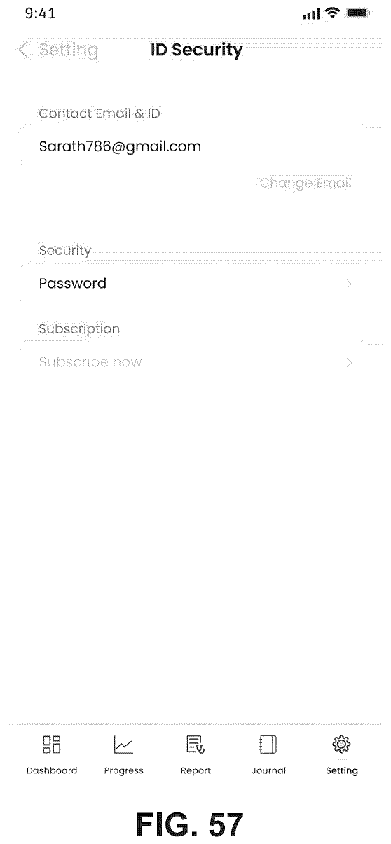

FIG. 57 shows a user interface for configuring ID security. A user can use said user interface for configuring/specifying the user's email address, wherein said email address can be used to contact and/or identify the user. A user can further use said user interface to update a password used for account access and/or to update a subscription plan associated with the user's account. A user can access said user interface using, for example, the interface shown in FIG. 54.

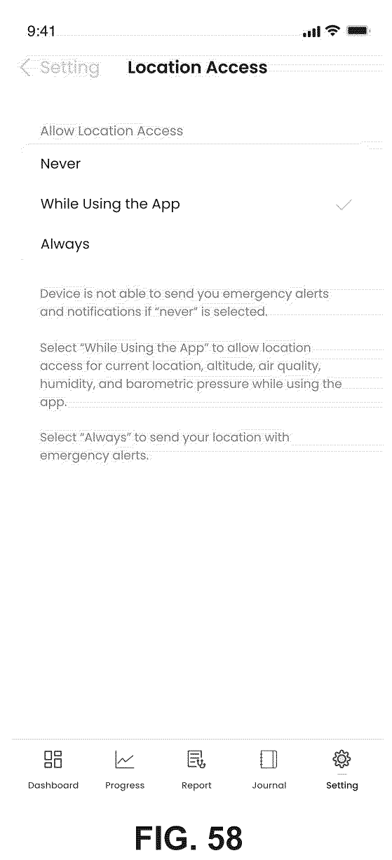

FIG. 58 shows a user interface for configuring location access in relation to a mobile app configured for use with one or more wearable devices. A user can configure location access to provide data indicating a user's geographic location to one or more programs related to a wearable device. A user can access said user interface using, for example, the interface shown in FIG. 54.

FIG. 59 shows a user interface for configuring a user profile. Said user interface can comprise a name field, gender field, birthday field, weight field, and/or height field. Said user interface can further be used to populate information about a primary user of one or more wearable devices. A user can access said user interface using, for example, the interface shown in FIG. 54.

FIG. 60 shows a user interface for configuring an SpO2 threshold for triggering an alert. A user can set said SpO2 threshold between a range of, for example, 92% and 97%. Said threshold can trigger, for example, the alert sub-window shown in FIG. 49. The user interface can include an interactive element such as a slider, drop-down menu, radio buttons, or any other graphical or text-based element, with which the user can interact to set the SpO2 threshold. A user can access said user interface using, for example, the interface shown in FIG. 54.

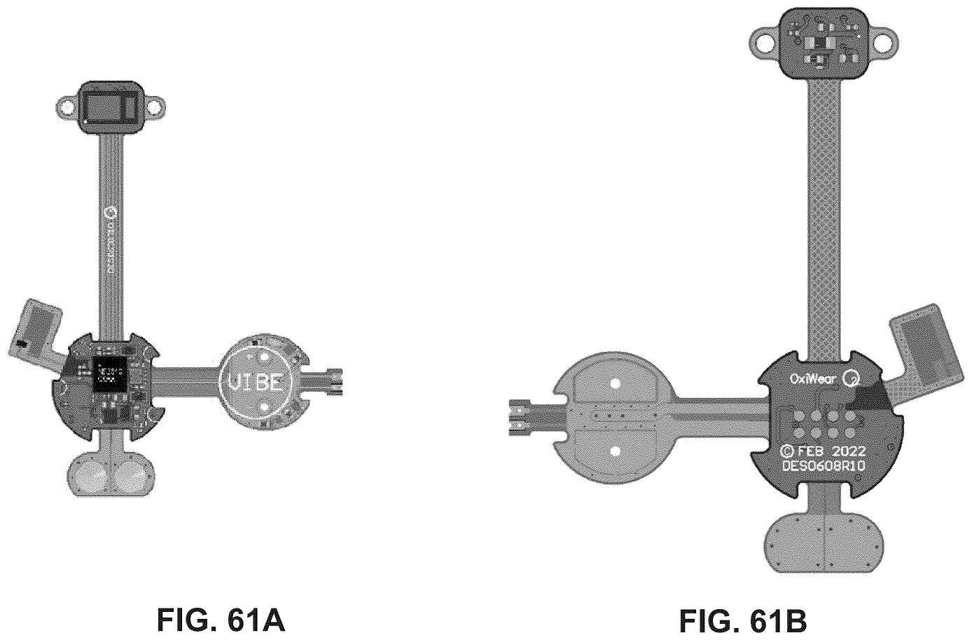

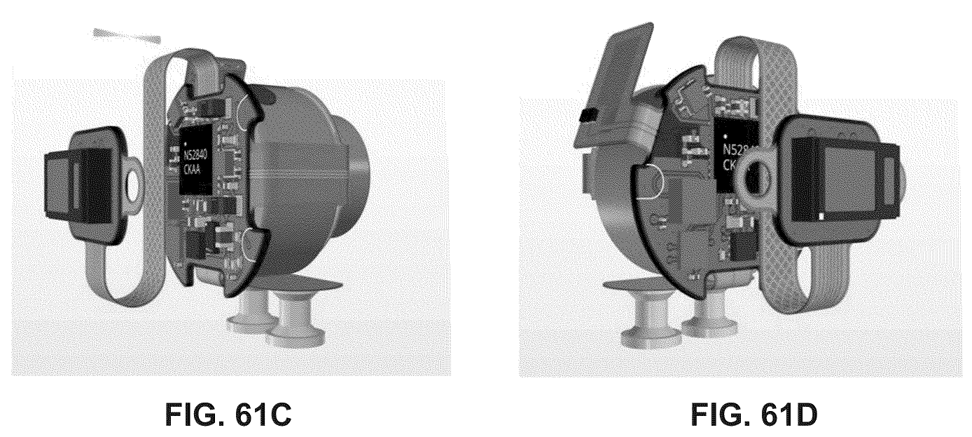

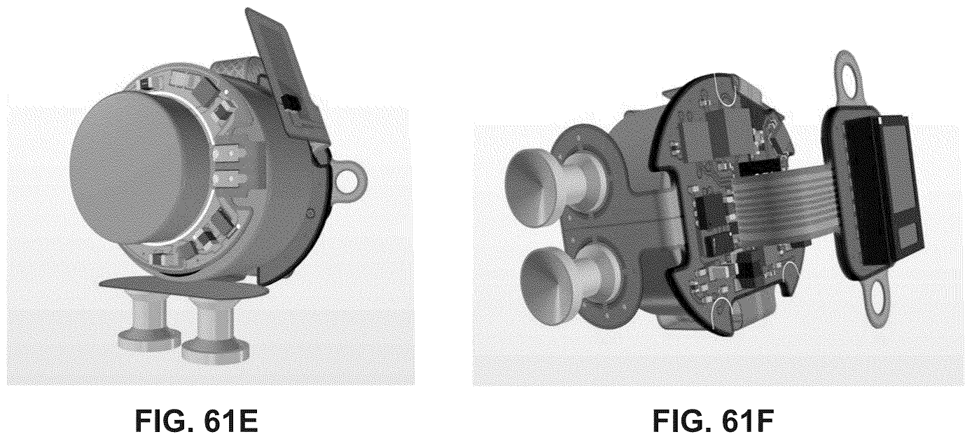

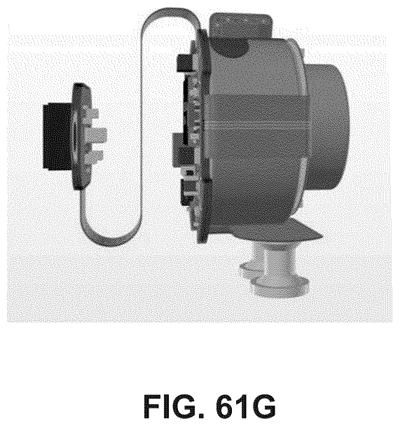

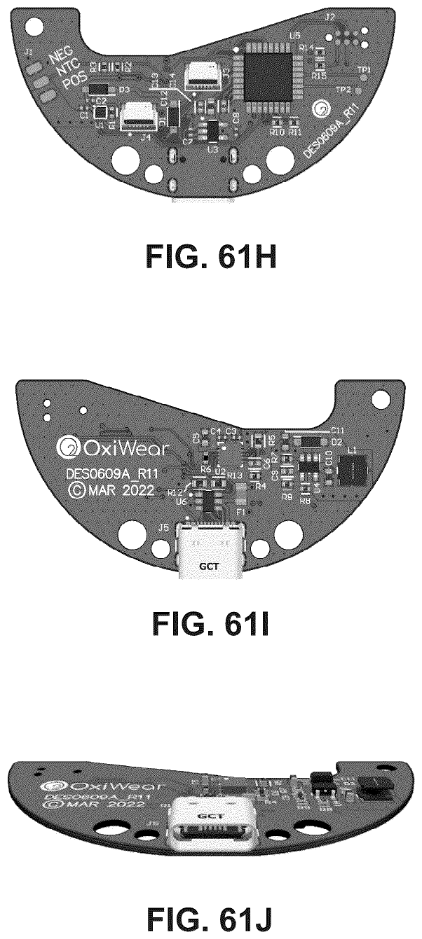

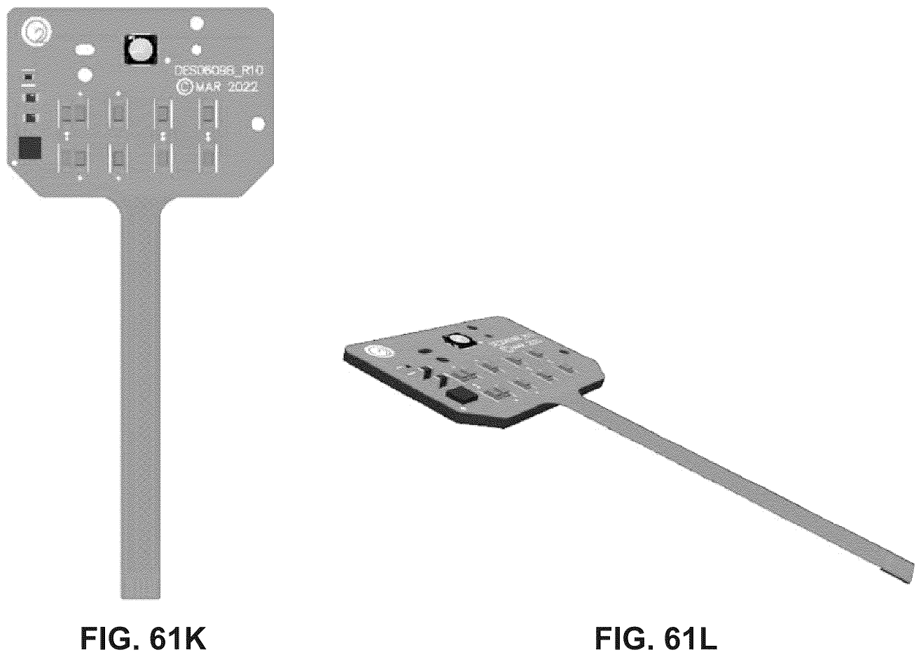

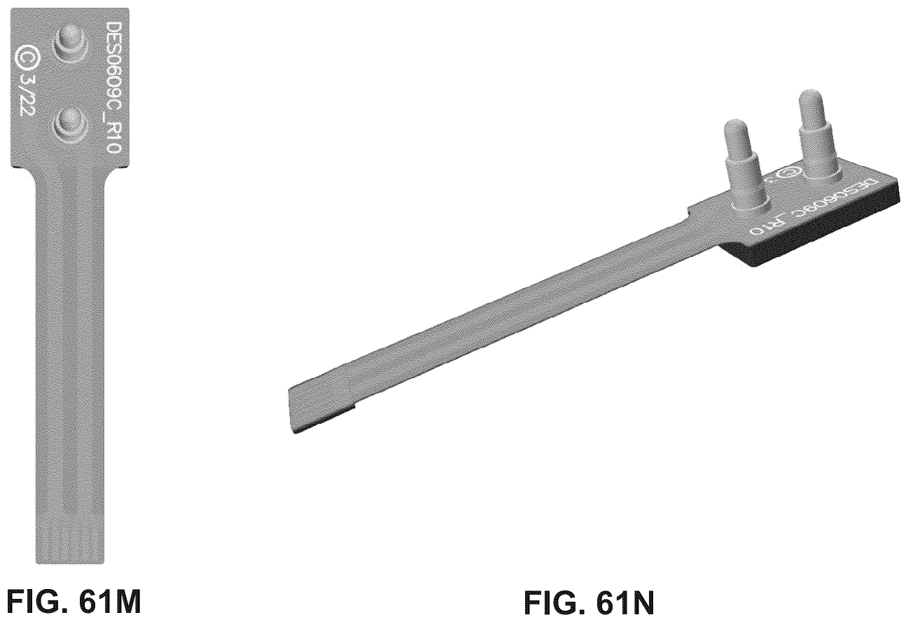

FIGS. 61A-61N include renderings of a flexible electronics assembly (e.g., electronics 760 of FIG. 7A-7C) for a wearable monitor, in various configurations, illustrating the foldability/flexibility of the assembly, as well as components thereof, according to some embodiments.

In some embodiments, a method of manufacturing an ear-wearable apparatus includes bonding an electronics board to an earpad using at least one of silicone or epoxy such that the electronics board is affixed to the earpad within a cavity defined in a surface of the earpad, and attaching the earpad to a chassis.

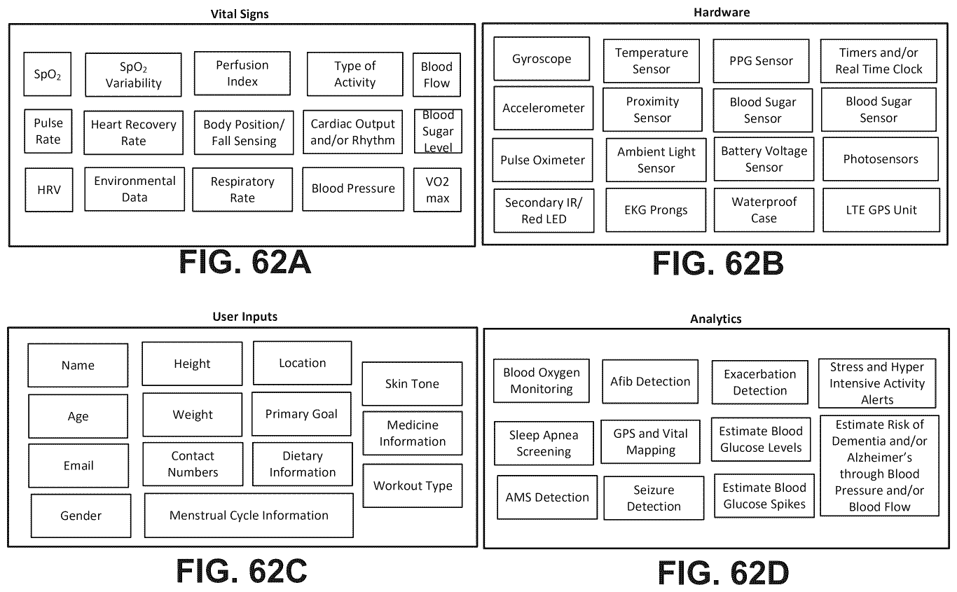

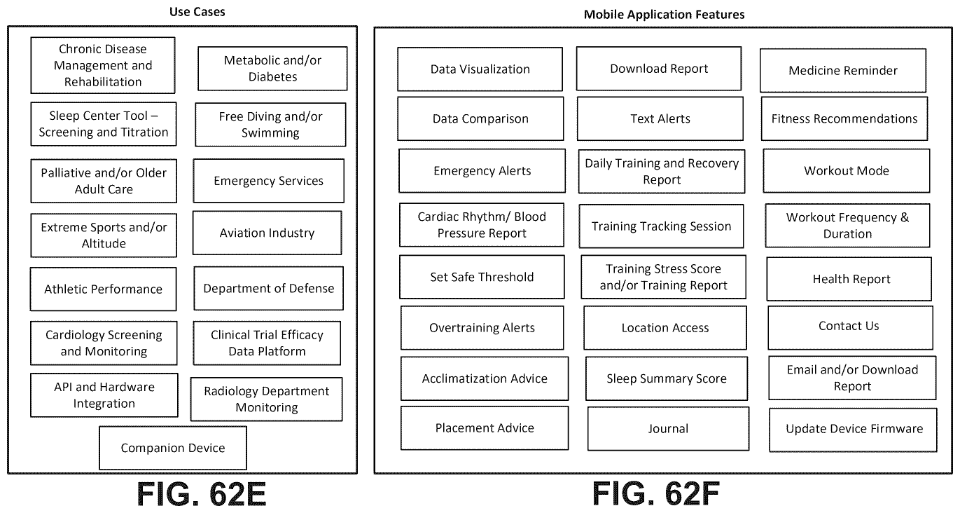

The wearable monitor described above, in accordance with any of the foregoing embodiments, may include one or more sensors and/or hardware components (e.g., device, apparatus) as illustrated in FIGS. 62A and 62B. Any combination of the sensors and hardware components shown in FIGS. 62A and 62B may be included depending on the specific embodiment or user requirements.

The wearable monitor can include at least one of a PPG sensor (e.g., a PPG sensor including IR and/or red LED, and a photosensor), an accelerometer (e.g., an accelerometer configured to detect motion, inclination, single taps, and double taps), a temperature sensor, a proximity sensor, an ambient light sensor, a timer, a real-time clock, secondary IR/red LED, gyroscope (e.g., a gyroscopic sensor), EKG prongs, a waterproof and/or wireless charging case (e.g., having IP 67/68), LTE (long-term evolution), GPS (global positioning system), or LTE-GPS unit. The wearable monitor, according to multiple embodiments disclosed herein, can be configured to provide continuous, semi-continuous, or intermittent real-time monitoring of various respiratory, physical, physiological and/or environmental data (e.g., altitude air quality, barometric pressure) as shown in FIG. 62B.

In some embodiments, the wearable monitor can be designed to increase its sampling rate to facilitate precise, quick, and detailed data collection. The form factor of the wearable monitor can be modified to accommodate this increased sampling rate, allowing for the integration of secondary IR/red LEDs, EKG prongs, LTS, and/or GPS units. The sampling rate of the wearable monitor can be set to 50 Hz, 60 Hz, 70 Hz, 80 Hz, 90 Hz, 100 Hz, or higher. Additionally, the wearable monitor can be equipped with EKG prongs.

In some embodiments, the wearable monitor can be configured to measure, detect, or estimate one or more parameters of SpO₂, pulse rate, HRV, SpO₂ variability, heart recovery rate, environmental data (e.g., altitude, air quality, and/or barometric pressure), VO₂ max, perfusion index, body position/fall sensing, respiratory rate, blood pressure, blood flow, type of activity, cardiac output/rhythm, blood sugar level, SpNO₂, SpCO, EKG, or sleep movements.

A wearable monitor designed for athletic performance monitoring may include accelerometers, temperature sensors, and heart rate sensors, whereas a model intended for general health tracking might include pulse oximeters, blood sugar sensors, and GPS tracking. The modularity of these components allows the wearable monitor to be customized or scaled to meet specific health monitoring needs. The wearable monitor described above, in accordance with any of the foregoing embodiments may further include a photoplethysmography (PPG) sensor including infrared (IR) and/or red light-emitting diodes (LEDs) paired with a photosensor.

In some embodiments, the wearable monitor may further include a temperature sensor, for example, to increase the accuracy of SpO₂ readings, especially under changing environmental or body conditions. In some embodiments, the wearable monitor may further include a proximity sensor. In some embodiments, the wearable monitor may further include at least one of an accelerometer and a gyroscopic sensor to monitor at least one of body position, movement, or respiratory patterns, which can be used to for fall detection and activity type recognition. In some embodiments, the wearable monitor may further include an ambient light sensor to automatically adjust the intensity of a LED output based on surrounding light conditions. In some embodiments, the wearable monitor may further include a battery voltage sensors. In some embodiments, the wearable monitor may further include lightweight and power-efficient components, such as wireless sensors, GPS modules, pressure sensors, voltage sensors, and real-time clocks. At least two of sensors and/or hardware components (e.g., device, apparatus) shown in FIG. 62A may operate in combination to monitor a set of vital signs and/or environmental conditions. In some embodiments, the use of these sensors and/or hardware components allows for continuous monitoring of health metrics such as heart rate variability, blood sugar levels, respiratory rate, and cardiac output. The combination of these sensors allows for a more accurate representation of vital signs by adjusting measurements based on external factors such as ambient temperature and light levels.

Referring to FIGS. 62A and 62B, the wearable health monitor, in accordance with any of the foregoing embodiments, may measure a wide range of vital signs including, but not limited to, SpO₂ (blood oxygen saturation), pulse rate, heart rate variability (HRV), SpO2 variability, and the perfusion index (PI). By leveraging PPG sensors and data from accelerometers, the wearable monitor can also track heart rate recovery (HRR), which can provide insights into cardiovascular health and fitness levels. The wearable monitor may further track environmental conditions, such as temperature and light exposure. In some embodiments, the wearable monitor can be configured to monitor body position and detect falls, which is particularly useful for elderly users or individuals at risk of sudden falls. In some embodiments, the wearable monitor may monitor the user's respiratory rate by analyzing both pulse wave data and body movements. In some embodiments, the wearable device can monitor vital signs such as cardiac output, blood flow, and/or blood sugar levels. In some embodiments, the wearable monitor can track the activity type. For example, the wearable monitor may distinguish between rest periods and physical activity. The ability to monitor a wide variety of vital signs, both individually and in combination, allows the device to provide a more accurate and holistic analysis of the user's physiological state. In some embodiments, the combination of heart rate variability, respiratory rate, and body movement can also be used to detect sleep patterns and provide detailed sleep reports.

The wearable health monitor, in accordance with any of the foregoing embodiments, may include one or more ECG/EKG contact point(s) (e.g., electrocardiogram prongs and/or probes) placed where the typical sensor contacts the ear which can be used to collect ECG data directly from the user. In some embodiments, the collected ECG/EKG data can be added as another metric for athletic performance monitoring. Alternatively, in some embodiments, the collected ECG/EKG data can serve as the sole metric for athletic performance monitoring.

In some embodiments, the wearable health monitor integrates multimodal sensors that cross-reference heart rate (HR) and blood oxygen saturation (SpO₂) values with photoplethysmography (PPG) derived values to improve reliability and efficiency. In some embodiments, the wearable health monitor implements matched filtering techniques such as deep match frameworks in combination with multimodal sensor integration to cross reference HR and SPO₂ with PPG derived values to improve reliability and efficiency.

Using matched filtering and deep match frameworks, the monitor can refine ECG signals, achieving heart rate and oxygen saturation data, which can be correlated with PPG-derived measurements to verify accuracy. This approach can provide a comprehensive health monitoring solution suitable for users looking for enhanced reliability through cross-validation of different data sources.

In some embodiments, the wearable health monitor can employs both electrocardiogram (ECG) and ballistocardiogram (BCG) measurements to offer a comprehensive view of the user's cardiovascular health. By placing an accelerometer near the ear, the monitor can track subtle head movements (e.g., an indirect BCG measurement source) capturing BCG's principal peaks (J-waves) synchronized with each heartbeat. Through ensemble averaging, the monitor can calculate stable J-wave amplitudes correlated with stroke volume, providing an insight into the user's cardiac output. This dual-modality configuration can be used for users seeking heart function data.

In some embodiments, the wearable health monitor can capture the ECG locally near the ear using a single-lead ECG setup. By combining ECG data with BCG signals, the monitor derives an RJ interval, an electromechanical duration that can be used in clinical and performance applications. To handle the low signal-to-noise ratio typical in this configuration, the monitor can apply cross-correlation techniques to statistically isolate the RJ interval, improving accuracy in monitoring of pre-ejection period (PEP) and heart rate.

In some embodiments, the wearable health monitor can include a custom integrated circuit optimized for (or designed to improve) low power consumption, drawing less than 60 nW (e.g., 58 nW), which reduces the need for frequent charging while maintaining performance. This circuit can replace traditional ECG component, such as the instrumentation amplifier and analog-to-digital converter, with a single, compact chip, capable of accurately detecting R-waves and other critical ECG markers at the ear.

In some embodiments, the wearable health monitor may provide vital sign assessments, such as VOₑ and cardiac output monitoring, contributing to comprehensive cardiovascular health monitoring and respiratory health monitoring, as well as exercise metrics and trends. Cardiovascular monitoring includes assessing cardiac rhythm and respiratory health during physical activities. Indirect VO₂ measurements use HR and HRV alongside RR to gauge oxygen efficiency. Machine learning (ML) or AI can refine these measurements, enabling more accurate cardiac output and VOₑ data during exercise. Further, in some embodiments, the wearable health monitor may analyze the perfusion index and oxygen saturation (SpO₂) levels for monitoring oxygen delivery and circulation, aiding in the detection of peripheral artery disease. Using trends in the perfusion index (PI) provides insights into blood flow and tissue oxygenation, supporting the detection of peripheral artery disease (PAD).

In some embodiments, the wearable health monitor may utilize tilt and fall sensing capabilities based on accelerometer and/or gyroscope data, enhancing the monitor's ability to detect falls and respond to emergencies effectively. This feature can support remote patient monitoring, enhancing safety with body position and fall detection functionalities.

In some embodiments, the wearable health monitor may also monitor respiratory rate through PPG waveform analysis, including assessments such as relative inspiratory amplitude variance (RIAV) and relative inspiratory flow variance (RIFV). RIAV can track changes in blood flow due to thoracic pressure variations that affect PPG amplitudes. RIFV measures shifts in PPG signal frequency over time, aiding in the estimation of long-term respiratory health trends.

In some embodiments, the wearable health monitor may analyze blood pressure through PPG waveform analysis for PTT evaluation. PPG waveform-derived pulse transit time (PTT) is used for non-invasive blood pressure monitoring. In some embodiments, the wearable health monitor may utilize PPG waveforms to assess blood flow and/or determine pulse wave velocity (PWV), thereby contributing to a comprehensive evaluation of cardiovascular health. User input-based algorithms can help identify specific activities, which in turn can optimize (or at least improve) data collection by adjusting for motion artifacts. For example, recognizing lateral versus vertical movements can fine-tune gain settings and data accuracy, improving calorie estimates and PPG signal fidelity.

In some embodiments, the wearable health monitor may estimate blood sugar levels indirectly through PPG measurements, or in combination with glucose monitoring devices, providing users with critical insights into their metabolic health. This approach enhances the versatility of the monitor by enabling metabolic health assessments through combined or standalone glucose monitoring systems.

In some embodiments, the wearable health monitor may further enhance data collection capabilities by employing dual PPG waveform analysis using the Beer-Lambert algorithm to assess SpNO₂ levels. Additionally, in some embodiments, the wearable health monitor may utilize a dual-wavelength approach, including 650 nm and 805 nm PPG wave analysis for oxyhemoglobin assessment. This method facilitates the monitoring of SpCO levels, as carboxyhemoglobin absorbs more red light at 650 nm and oxyhemoglobin reacts more at 805 nm, enabling continuous monitoring for early carbon monoxide exposure detection and preemptive safety measures.

In some embodiments, the wearable health monitor may perform direct electrocardiogram (EKG) measurements obtained through EKG prongs and/or electrodes. These hardware elements enhance precision in direct EKG signal capture, reinforcing the device's capability for comprehensive cardiac monitoring.

In some embodiments, the wearable health monitor may be configured to monitor sleep movements using an accelerometer and gyroscope algorithm to detect motion throughout the night. This monitoring can be further expanded by categorizing moments of increased HR and HRV in conjunction with movement to signal potential instances of poor sleep quality and generate a comprehensive sleep score. Referring to FIG. 62C, the wearable monitor, in accordance with any of the foregoing embodiments, can allow users to input personal data to enhance the accuracy and/or personalization of health monitoring. Users may provide information, as shown in FIG. 62C, such as their name, gender, age, height, weight, skin tone, dietary information, medicine information, menstrual cycle information (e.g., for menstrual cycle tracking), location, email, contact numbers, primary goal, work out type, and/or the like, which the wearable monitor can use to adjust sensor readings accordingly. For example, skin tone is a factor in photoplethysmography (PPG) measurements, as varying melanin levels affect light absorption. By allowing users to input their skin tone, the device can adapt its algorithms to ensure accurate readings of SpO₂ and pulse rate across different skin types, enhancing overall accuracy.

The wearable monitor may also accept inputs related to health goals, such as fitness, recovery, or general wellness. Users can input data such as their primary goals, dietary preferences, and medication schedules, enabling the device to provide personalized health recommendations, medication reminders, and fitness tracking. Menstrual cycle information may also be included, allowing the wearable monitor to offer tailored health insights based on hormonal fluctuations. In some embodiments, the wearable monitor may also collect location data via GPS, which can be used to provide environmental health recommendations, such as acclimatization advice when traveling to high altitudes.

Furthermore, user input may extend to contact information and emergency details, enabling proactive health management. For instance, in the case of a fall detection event or other health anomalies, the wearable monitor may automatically notify emergency contacts or healthcare providers. The combination of user input and real-time data from the sensors allows for personalized feedback, making the device more effective at addressing the specific health needs of the wearer.

In some embodiments, the wearable health monitor may be configured to allow users to provide dietary information input, such as nutritional intake. This data can be processed by the device to provide tailored health feedback that aligns with the user's unique metabolic profile and health objectives.

In some embodiments, the wearable health monitor comprises a multi-wavelength optical sensor system configured to enhance measurement accuracy across various skin tones, including darker skin tones. Accordingly, in some embodiments, the wearable health monitor is configured to allow users to provide their skin tone. By using red, IR, green, and blue wavelengths during device initialization, the returned PPG signals from each wavelength can be compared. These values allow a Fitzpatrick Scale value to be assigned to each user. Using this scale, heart rate (HR), SpO₂, and heart rate variability (HRV) values can be optimized (or improved) to better calibrate the algorithm, ensuring it adapts accurately to various skin tones.

In some embodiments, the wearable health monitor is configured to allow users to enter their workout type. In such embodiments, the wearable health monitor can be operable to provide comprehensive workout tracking capabilities, including calorie tracking and activity-based tracking. The wearable health monitor may record user progress across various types of physical activities, thereby facilitating personalized fitness insights and enhancing user engagement. In some embodiments, similar to athletic performance monitoring, where accelerometer and gyroscope data, along with heart rate (HR) values, are used to estimate calorie tracking, an athletic training dashboard can be provided. This dashboard would showcase the user's performance through daily and lifetime metrics, utilizing various parameters.

In some embodiments, the wearable health monitor is configured to allow users to enter their medicine information. Accordingly, in some embodiments, the wearable health monitor may be configured to include medication monitoring capabilities, allowing users to input their medication schedules. In some embodiments, the wearable health monitor can analyze trends in heart rate and/or other physiological metrics in correlation with the recorded medication data, thereby enabling the detection of potential physiological responses or adverse effects. Heart rate (HR) and heart rate variability (HRV) trends can be synced with the timing of medication intake and the onset of its effects. This synchronization can be used to track side effects and other metrics, creating a comprehensive medical profile. Additionally, patients can be reminded to take their medications on time through vibrations and phone alerts.

In some embodiments, the wearable health monitor is configured to allow users to provide their menstrual cycle information. In some embodiments, the wearable health monitor is operable to track menstrual cycle phases, correlating such data with heart rate and heart rate variability trends. This functionality can provide users with insights into the effects of the menstrual cycle on their physiological performance and overall well-being. In some embodiments, the wearable health monitor tracks the menstrual cycle by analyzing heart rate (HR) and heart rate variability (HRV) trends. Continuous HRV monitoring can reveal patterns, with higher HRV indicating the follicular phase and lower HRV suggesting the luteal phase. These HRV values are compared to baseline metrics to identify the different phases of the menstrual cycle.

Referring to FIG. 62D, the wearable monitor, in accordance with any of the foregoing embodiments, is configured to receive, store and/or produce analytics for at least one of sleep apnea screening, acute mountain sickness (AMS) detection, estimate risk of Dementia and/or Alzheimer's disease through blood pressure and/or blood flow, estimate blood glucose and/or blood glucose spikes, exacerbation detection, Afib (atrial fibrillation) detection, stress and/or hyper intensive activity alerts, seizure detection, or GPS and/or vital mapping.

For example, the wearable monitor can be capable of continuously, semi-continuously, or intermittently tracking SpO₂ variability to detect issues such as sleep apnea or AMS. By integrating data from multiple sensors, such as HRV and respiratory rate, the wearable monitor may also detect cardiovascular irregularities, including atrial fibrillation (Afib).

In some embodiments, the wearable health monitor may also track blood glucose levels and estimate spikes based on physiological data collected from the user. The wearable monitor can integrate GPS data to offer vital sign mapping, providing context to measurements such as heart rate and respiratory rate during physical activities. The wearable monitor can also track stress levels and hyper-intensive activities by analyzing heart rate variability, body movement, and respiratory patterns. These insights can be useful for managing fitness performance and identifying signs of over-training or stress.

In some embodiments, the wearable monitor may also be configured to detect/estimate health risks associated with elderly users, such as dementia, Alzheimer's disease, Parkinson's disease, by continuously, semi-continuously, or intermittently monitoring movement patterns, cognitive responses, heart rate variability, and/or the like.

In some embodiments, the wearable health monitor can identify sleep apnea episodes, and issue alarms, and hypoxia alerts for early intervention. In some embodiments, issuing alarms and alerts is similar to sleep center tools described herein but further involves implementing monitoring and alarm thresholds, which could also be used in conjunction with continuous positive airway pressure (CPAP) devices and other treatment methods. Accordingly, in some embodiments, the wearable health monitor can implement monitoring and alarm thresholds, that can be used in conjunction with CPAP devices and other devices to ensure proper airflow is provided to the patient, and if the levels are below the threshold the device can alarm and wake up the patient.

In some embodiments, the wearable health monitor can detect acute mountain sickness (AMS) and/or altitude sickness by monitoring oxygen levels and issuing hypoxia and/or oxygen saturation alerts. In some embodiments, the wearable health monitor can continuously measure SpO₂ levels and notifies users when thresholds indicating potential hypoxia are reached. In some embodiments, the wearable health monitor may provide alerts at specific thresholds based on the patient's SpO₂ variability values. This feature can ensure that the patient is able to increase altitude safely by monitoring and maintaining these thresholds. If the patient begins to ascend in elevation too quickly, the device is configured to alert the climber, thereby promoting safe acclimatization practices.

In some embodiments, the wearable health monitor can estimate the risk of dementia and Alzheimer's by analyzing blood pressure and/or blood flow. For example, in some embodiments, the wearable health monitor can estimate the risk of dementia and Alzheimer's by analyzing blood pressure variability (BPV), pulse wave velocity (PWV), and pulse transit time (PTT), along with optionally issuing blood pressure alerts. The monitor can track these metrics and provide insights to detect early signs of reduced cognitive function.

In some embodiments, blood pressure variability (BPV) may be measured indirectly by monitoring pulse wave patterns and their variations over time, allowing for the logging of baseline values and the flagging of irregularities, which could potentially indicate long-term risks of dementia. Additionally, pulse wave velocity (PWV) readings can provide insights into atrial health; high PWV readings over time suggest reduced elasticity in blood vessels, serving as another indicator for patients at risk of dementia and cognitive decline.

In some embodiments, the wearable health monitor can estimate blood glucose levels and detect spikes through PWV, PTT, hyperglycemia alerts, and BPV monitoring. In some embodiments, the monitor can provide real-time feedback on blood glucose fluctuations and issues warnings when spikes occur. Similar to cases with diabetes, increased PWV suggests elevated glucose levels. While PTT and BPV are indirect indicators of glucose spikes, a shortened PTT corresponds to elevated blood pressure, another byproduct of high glucose levels. Rapid spikes in PTT and PWV can indicate glucose-induced shifts, especially when these metrics deviate from the user's baseline trends over time.

In some embodiments, the wearable health monitor can identify potential exacerbations by monitoring blood pressure, BPV, and PWV. In some embodiments, the monitor issues alerts for blood pressure spikes (e.g., dangerous increases in blood pressure) to prompt early medical attention. Calculating SpO₂ entropy using SpO₂ levels can effectively detect exacerbations in users, particularly those with chronic diseases. By monitoring these levels alongside normal thresholds, the device can provide real-time alerts through vibrations or notifications. This enables users to take preventive actions such as resting, taking medication, or contacting medical assistance promptly.

In some embodiments, the wearable health monitor can detect atrial fibrillation (AFib) by monitoring irregular heartbeats and/or irregular heart rate, issuing alerts for abnormal heart rhythms to facilitate timely medical response. Additionally, the monitor can measure pulse wave velocity (PWV) and/or pulse transit time (PTT) to provide comprehensive cardiovascular monitoring, for example to detect AFib. Using PPG and ECG, abnormal heart rhythms and irregular heart rate patterns can be detected through continuous patient monitoring. By determining baseline heart rate (HR) and heart rate variability (HRV) trends, these technologies can effectively identify deviations. Additionally, R-R intervals can trigger threshold detection algorithms, enhancing the accuracy of these alerts.

In some embodiments, the wearable health monitor can track stress indicators and issue hypertensive or stress alerts to help users manage high activity levels. The monitor can provide warnings when stress levels become potentially harmful. Similar to chronic disease management and exacerbation detection, a rapid increase in blood pressure (BP) beyond the threshold and PPT can be used to notify the user to relax, take medication, or seek medical assistance.

In some embodiments, the wearable health monitor can detect potential seizures or seizures by monitoring heart rate, bradycardia, PWV, PTT, and/or abnormal HRV, with optionally issuing alerts when seizure-like anomalies are identified. By using continuous heart rate (HR) monitoring, thresholds can be set to indicate bradycardia or tachycardia. Additionally, pulse wave velocity (PWV) can be utilized for seizure detection. Monitoring sharp increases in PWV trends can provide early warning signs of cardiovascular stress, which may indicate an impending seizure, allowing timely notifications to the patient.

In some embodiments, the wearable health monitor can integrate GPS with vital signs, offering users and emergency services real-time health data and location information during critical alerts.

Referring to FIG. 62E, the wearable monitor, in any of the foregoing embodiments, can be configured to serve a wide variety of purposes across healthcare, sports, and industry. The wearable monitor can be configured for use in various applications, including sleep center tools (e.g., screening and/or titration), chronic disease management and rehabilitation, palliative and/or older adult care, extreme sports and/or high-altitude activities, athletic performance, cardiology screening and/or monitoring, companion devices, API (application programming interface) and hardware integration, metabolic and/or diabetes management, emergency services, the aviation industry, the Department of Defense, clinical trial efficacy data platforms, radiology department monitoring, and free diving and/or swimming. For example, in some embodiments, the wearable monitor can track the vital signs of pilots, astronauts, and/or military personnel, where it may be used to detect early signs of hypoxia or altitude-induced stress during high-stakes missions. By tracking metrics in real time, the monitor can help ensure safety in extreme conditions, such as high altitudes and confined environments.

In chronic disease management and rehabilitation, it can offer monitoring of vital signs such as blood oxygen levels, heart rate, respiratory rate, and blood pressure, enabling real-time tracking for individuals managing conditions like diabetes or cardiovascular issues. In sleep centers, the wearable monitor can be employed for screening and titration, detecting issues like sleep apnea.

In some embodiments, the wearable monitor can be used by athletes to optimize (or improve) training duration or intensity while avoiding overtraining or injury. This functionality is particularly useful in high-performance environments like extreme sports and free diving/swimming, where oxygen levels and cardiovascular stability are critical. The wearable monitor can provide real-time feedback that can help users adjust their efforts during demanding activities, ensuring both safety and peak performance. In some embodiments, the wearable monitor can be configured for use in the aviation industry, where it tracks the vital signs of pilots and astronauts, detecting early signs of hypoxia or other stressors caused by high altitudes, enhancing safety during long flights or space missions.

In some embodiments, the wearable monitor can monitor at least one of vital signs, detect falls, or track respiratory function, providing caregivers with updates that can improve care for elderly people. Emergency services can also benefit from its real-time health tracking capabilities, ensuring that first responders' vitals are monitored during critical situations. In some embodiments, the wearable monitor can be implemented in clinical trials to collect data on treatment efficacy, acting as a platform for gathering ongoing health information. In some embodiments, the wearable monitor can be used in radiology departments, where it can monitor patients during imaging procedures, ensuring vital signs remain stable during potentially stressful or prolonged scans.

Using SpO₂, respiratory rate (RR), HR and overnight vital monitoring can be used to screen patients for sleep apnea and telehealth monitoring of patients with sleeping disorders. High HRV variability and SPO₂ desaturations events can be used to detect significant sleep stage transitions. While sleep apnea could be detected using HRV, HR, RR, SPO₂ to classify sleep apnea severity (e.g., mild, moderate and severe), for example setting a SPO₂ threshold and pair it with HR spiking and HRV drops to indicate various degrees of sleep apnea. Accordingly, in some embodiments, the wearable monitor can assist in screening and titration for sleep disorders, such as sleep apnea, using SpO₂ and heart rate (HR) to evaluate sleep apnea severity, for example by classifying it into mild, moderate, and severe cases. In some embodiments, the wearable monitor can monitor HRV variability and SpO₂ desaturation events overnight to detect sleep stage transitions. Telehealth data may enable remote monitoring of sleep disorders.

In some embodiments, the wearable monitor can be used for chronic disease management, such as peripheral artery disease (PAD), arrhythmia, and hypertension. Real-time monitoring of blood oxygen, heart rate, respiratory rate, and blood pressure can provide continuous data, aiding individuals with conditions like diabetes and cardiovascular disease. In PAD, for instance, delayed pulse arrival times (PAT) can signal obstructed blood flow, with baseline PAT values compared during various exercises to assess PAD severity.

PAD detection can be achieved by analyzing changes in blood flow and waveforms, along with arrhythmia detection and hypertension management. PAD detection involves monitoring pulse wave forms and comparing pulse arrival times PAT between heartbeats. PAD is exhibited by delayed pulse arrival times due to obstructed blood flow. Using baseline values of PAT and compared to low level and high level exercises to differentiate between various degrees of PAD severity.

In some embodiments, fall risk assessment can be performed by tracking balance, stability, and HRV. A rapid drop in blood pressure and other metrics, along with body tracking to show imbalance within the patient, can be used. In some embodiments, the wearable monitor may include functions for at least one of fall risk assessment, stress management, pain detection, or telehealth monitoring. By tracking balance, HRV, and blood pressure, the monitor can assist in assessing fall risk, as sudden blood pressure drops combined with stability data may reveal imbalance. An accelerometer can detect falls and notifies caregivers, supporting responsive care for older adults. For example, spikes in the accelerometer can detect falls and notify the caregiver or patient caretaker.

In some embodiments, the wearable health monitor can be used for tracking oxygen saturation, altitude acclimation, and hypoxia during high-altitude sports. This can reduce serious health risks at high altitudes by assessing oxygen saturation using SPO₂ to prevent altitude sickness or acute hypoxia. Hypoxia can be detected and managed by monitoring SpO₂ levels, which mitigates health risks at high altitudes associated with altitude sickness or acute hypoxia.

Optimized exercise frequency, HRV tracking, HR monitoring metrics can all be combined with a multimodal analysis of the user given that the wearable monitor can optimize (or improve) exercise frequency to avoid overtraining, and promote peak performance using HRV patterns to gauge this. For example, consistent HRV increase after workout signals the athlete has recovered adequately. In addition, setting up a long term baseline of the athlete's HRV and HR metrics before and after workouts helps determine proper athletic training. Athletes can also track HRV and exercise frequency to balance performance with safety; for example, long-term HRV trends may indicate recovery status, helping to prevent overtraining.

In some embodiments, the wearable health monitor can be used for at least one of hypertension detection, CAD detection, heart failure/deterioration monitoring, and/or the like. Monitoring hypertension can be done through PWV, which correlates to arterial stiffness which is a common effect of hypertension where there baselines are compared to times of high stress. CAD detention can be detected by diminished HRV and irregular HR patterns due to limited blood supply such as the case with detecting pulse amplitude variations where a drop or significant delay in pulse waves can indicate reduced blood flow. Accordingly, in some embodiments, the wearable monitor can aid athletic performance optimization (or improvement) by analyzing HRV, HR, and multimodal data to support recovery and prevent injury. Baseline HRV trends before and after exercise may help determine desired recovery times. For cardiovascular screening, the wearable monitor may also detect conditions like hypertension, coronary artery disease (CAD), and heart failure. In some embodiments, diminished HRV and irregular HR patterns can indicate CAD due to reduced blood flow, making the monitor suitable for early detection and prevention efforts.

A chronic illness monitor, machine learning, and pairing with other devices, such as a glucose monitor or electrolyte patch, can be integrated into the wearable health monitor. This integration allows the device to act as a hub, consolidating data from each of the sensors and displaying it uniformly on the dashboard. This can provide a much more holistic view of the patient. Monitoring glucose along with heart rate (HR) and heart rate variability (HRV) can be used to categorize the patient's activities, such as eating meals, and flag anomalies preemptively. This allows patients to react proactively to potential flare-ups in their conditions. Accordingly, in some embodiments, the wearable health monitor may serve as a companion device by integrating with other sensors through API and hardware. This feature can support machine learning applications for chronic illness monitoring, and by pairing with devices such as glucose monitors, it can create a comprehensive health dashboard. For example, correlating HR and HRV with glucose levels may enable early anomaly detection, helping users manage chronic conditions.

Blood glucose trends cannot be identified under the current metrics. Using a continuous glucose monitor to track glucose trends alongside HRV and HR trends, however, can flag and integrate data across multiple metrics. This approach can aid in pre-hyperglycemia detection and remote patient monitoring. In some embodiments, the wearable health monitor can support metabolic and diabetes management by tracking blood glucose trends and/or early hyperglycemia indicators. By pairing with a continuous glucose monitor (CGM), the monitor can integrate multiple metrics for a holistic health profile, alerting users to potential complications in advance.

In some embodiments, the wearable health monitor can provide options of training optimization (or improvement), HRV monitoring, breath training, and other techniques, similar to those used for enhancing athletic performance, for divers (e.g., free divers) and/or swimmers. Additionally, monitoring safe SpO₂ levels is crucial, especially during free diving expeditions, to ensure safe oxygen levels. The wearable health monitor, according to multiple embodiments described herein, can provide early monitoring for users when their oxygen levels are low, promoting safety throughout their activities.

In some embodiments, the wearable monitor can be used for safety and performance optimization (or improvement) of free diving and/or swimming by tracking SpO₂, HRV, and breathing (e.g., breathing frequency). The wearable monitor can monitor safe oxygen levels during prolonged dives, alerting users to hypoxia risk and helping them adjust breathing techniques during dives or training sessions.

Referring to FIG. 62F, the mobile software application, according to any of the embodiments, is configured for use with one or more wearable monitors of the present disclosure and encompasses at least one of the features/functionalities shown in FIGS. 62A-E. The mobile software application can include features such as data visualization (e.g., live SpO₂% HR graph and measurements), data comparison (e.g., live graph and measurements comparison), emergency alerts based on fall sensing and/or vitals, cardiac rhythm/blood pressure report, set safe threshold, alert and notifications (alert users when their SpO₂ drops below the threshold), over training alerts, acclimatation advice, download report, text alerts (text the emergency contacts when the user is in emergency), daily training and recovery report, email report, training tracking session, training stress score, location access, sleep summary score, medicine reminder, journal, training report (e.g., calculate calories, VO2), placement advice (optionally including causing haptic feedback, vibration, sound(s), visual representation(s) such as graphics, or other indications via a GUI of the mobile software application, to convey to a user when their placement of the wearable monitor on their ear is correct, incorrect, improving, etc.), fitness recommendations, or workout frequency and/or duration. In some embodiments, the mobile software application can include alerts and notifications (e.g., alerting users when their SpO₂ drops below a threshold, overtraining alerts, acclimatization advice).

In some embodiments, the mobile software application can notify the user to reposition the device in their ear for improved signal reception.

The features can include data management tools such as download report and email report, which can allow users to generate and share detailed health and performance reports with healthcare professionals. The application may also provide contact us option for customer support and the ability to update device firmware. The mobile software application can include data visualization displaying health metrics through user-friendly graphs and charts and/or data comparison. In some embodiments, the mobile software application can include access location option to allow users track activity in relation to geographic data.

Health monitoring capabilities can also be incorporated into the mobile software application, with features such as emergency alerts for critical health changes and/or cardiac rhythm/blood pressure for detailed cardiovascular insights. The mobile software application can be configured to provide sleep summary score, daily training and/or recovery report. In some embodiments, users can receive medicine reminders via the mobile software application. In some embodiments, users can receive overtraining alerts to prevent injuries. For athletes, training reports (e.g., daily training reports), recovery reports (e.g., daily recovery reports) and/or training stress scores can be provided, in some embodiments. In some embodiments, users can receive fitness recommendations based on individual health metrics. In some embodiments, the mobile software application can further include workout mode settings for specific activities, workout frequency and/or duration tracking, and/or acclimatization advice for those in extreme conditions.

In some embodiments, the wearable health monitor may include emergency alert features based on fall sensing and vital sign monitoring. The monitor may utilize an accelerometer and/or gyroscope to detect rapid changes in movement, alerting for falls and subsequently notifying emergency services or designated contacts to facilitate assistance.

In some embodiments, the wearable health monitor may be configured to provide overtraining alerts by analyzing HRV baselines, indicating potential overtraining. This capability can enable the user to take preventive measures to mitigate the risk of overtraining-related injuries or fatigue.

In some embodiments, the wearable health monitor may also generate a daily training and recovery report, which includes a training stress score. This score quantifies the physiological impact of training sessions, allowing users to manage their fitness regimens effectively.

In some embodiments, the wearable health monitor may include a medicine reminder feature. This feature can prompt users to take their medications at scheduled times, thus promoting adherence to prescribed treatment plans.

The wearable health monitor may also incorporate placement advice through a mobile application, using signal-to-noise ratio (SNR)-based optimized device placement recommendations. Additionally, the monitor may employ vibration alerts to assist users in achieving optimal device placement for accurate readings. Using the SNR values output, set a baseline where values over 18 dB indicate great signal quality. The monitor can display a visual indicator to guide users in adjusting the device's position. As the SNR values increase or decrease based on the position, users can fine-tune their placement with each use.

In some embodiments, the wearable health monitor may provide fitness recommendations based on user data. These recommendations may include insights on workout frequency and duration, enhanced through the analysis of HRV and heart rate (HR) trends in comparison to baseline levels.

The device may also generate a cardiac rhythm and blood pressure report, providing users with detailed information on cardiovascular health. This data can be obtained through advanced algorithms that analyze photoplethysmography (PPG) waveforms to assess blood pressure using pulse transit time (PTT).

In some embodiments, the wearable health monitor may offer acclimatization advice by utilizing resting heart rate and HRV data collected at different altitudes. By incorporating location tracking, the device can establish distinct baselines corresponding to various elevations, providing users with actionable insights to support physiological adaptation.

In some embodiments, an apparatus includes a wearable device configured to be positioned on an ear of a wearer The wearable device includes a first body portion, a second body portion, and a connection member mechanically coupled to each of the first body portion and the second body portion, at least one of the first body portion or the second body portion having a processor, a memory operably coupled to the processor, and a sensor set including at least one of a pulse oximeter, a photoplethysmography (PPG) sensor, an accelerometer, a temperature sensor, a proximity sensor, an ambient light sensor, or a gyroscopic sensor, the memory storing instructions to cause the processor to at least two of: calculate a blood oxygen saturation level of the wearer; calculate a pulse rate of the wearer; calculate a blood pressure level of the wearer; calculate a blood flow rate of the wearer; identify a cardiac output/rhythm of the wearer; calculate a blood sugar level of the wearer; calculate a blood carbon monoxide level of the wearer; calculate a blood nitrous oxide level of the wearer; generate an electrocardiogram associated with the wearer; estimate a risk of one of dementia or Alzheimer's disease of the wearer by analyzing at least one of blood pressure variability (BPV), pulse wave velocity (PWV), and pulse transit time (PTT); or detect peripheral artery disease (PAD) of the wearer based on a pulse wave form detected via the wearable device.

In some implementations, the memory stores instructions to cause the processor to at least three of: calculate a blood oxygen saturation level of the wearer; calculate a pulse rate of the wearer; calculate a blood pressure level of the wearer; calculate a blood flow rate of the wearer; identify a cardiac output/rhythm of the wearer; calculate a blood sugar level of the wearer; calculate a blood carbon monoxide level of the wearer; calculate a blood nitrous oxide level of the wearer; generate an electrocardiogram associated with the wearer; estimate a risk of one of dementia or Alzheimer's disease of the wearer by analyzing at least one of BPV, PWV, and PTT; or detect PAD of the wearer based on a pulse wave form detected via the wearable device. For example, the memory may store instructions to cause the processor to perform three, four, five, six, seven, eight, nine, ten, or all eleven of the foregoing. Alternatively or in addition, the sensor set can include at least two of a pulse oximeter, a PPG sensor, an accelerometer, a temperature sensor, a proximity sensor, an ambient light sensor, or a gyroscopic sensor. For example, the sensor set can include two, three, four, five, six, or all seven of the foregoing.

In some implementations, the wearable device also includes a first light-emitting diode, a second light-emitting diode, and at least one optical sensor. The first light-emitting diode can be configured to emit light having a first wavelength. The second light-emitting diode can be configured to emit light having a second wavelength different from the first wavelength. The at least one optical sensor can be configured to detect a reflected portion of the first light and a reflected portion of the second light from a surface of the ear of the wearer. The first wavelength can be in a red light spectrum and the second wavelength can be in an infrared spectrum, and the processor can be configured to calculate the blood oxygen saturation level of the wearer based on a Beer-Lambert law and using an amount of light absorbed by the surface of the ear of the wearer.

In some implementations, the sensor set includes the PPG sensor, and the PPG sensor is configured to detect the pulse wave form based on measured time intervals between heartbeats. The PPG sensor can include one or more light-emitting diodes (LEDs) configured to emit light at or toward a tissue site of the ear of the wearer, and a photodiode configured to measure an intensity of non-absorbed light reflected from the tissue site. The one or more LEDs can include at least one of a red LED, a green LED, or a yellow LED.

In some implementations, the sensor set also includes at least one of an accelerometer or a gyroscopic sensor and is configured to detect at least one of a body position, a head position, or a fall event of the wearer.

In some implementations, the wearable device also includes a first light-emitting diode, a second light-emitting diode, and a sensor set. The first light-emitting diode can be configured to emit light having a first wavelength, the second light-emitting diode can be configured to emit light having a second wavelength different from the first wavelength, and the sensor set can also include an ambient light sensor. The apparatus can be configured to adjust an intensity of light emitted by the first light-emitting diode and the second light-emitting diode based on detected ambient light conditions.

In some implementations, the sensor set also includes a proximity sensor configured to detect at least one of a position or an orientation of the wearable device relative to the ear of the wearer.

In some implementations, the wearable device also includes a vibration motor operably coupled to the processor, the memory storing instructions to cause the processor to operate the vibration motor in response to detecting an alarm condition.

In some implementations, the wearable device also includes a wireless transceiver operably coupled to the processor, and configured to communicate with a mobile software application. The memory can store instructions to cause the processor to transmit measurement data to the mobile software application via the wireless transceiver using at least one of Bluetooth®, LTE, or Wi-Fi protocol.

In some implementations, the memory stores instructions to cause the processor to generate an alert when the calculated blood oxygen saturation level is below a predefined threshold, the alert comprising at least one of an audible sound, a vibration, or a visual indication.

In some implementations, the wearable device also includes an alert mechanism operably coupled to the processor, the memory storing instructions to cause the processor to initiate an emergency plan in response to detecting a user interaction with the alert mechanism. The emergency plan can include at least one of: initiating a telephone call to emergency services, sending a short message service (SMS) message to a predefined contact, or transmitting an alert signal to a mobile software application.

In some implementations, the memory further stores instructions to cause the processor to estimate a risk of dementia or Alzheimer's disease by analyzing at least one of blood pressure variability, pulse wave velocity, or pulse transit time detected via the sensor set.

In some implementations, the wearable device also includes a temperature sensor configured to measure a skin temperature of the wearer, the wearable device configured to recalculate the blood oxygen saturation based on the measured temperature.

In some implementations, the wearable device also includes a battery voltage sensor, and the wearable device is configured to regulate a power consumption by adjusting an operation of the sensor set based on an activity level of the wearer and a battery voltage detected by the battery voltage sensor.

In some implementations, the memory further stores instructions to cause the processor to generate a sleep report based on data collected from at least two sensors from the sensor set.

In some implementations, the wearable device also includes at least one microphone and at least one speaker operably coupled to the processor, the memory storing instructions to cause the processor to activate the microphone and the speaker in response to initiating an emergency plan.

In some implementations, the wearable device is configured to continuously monitor a blood oxygen saturation level of the wearer and to dynamically adjust a sampling rate of the sensor set based on detected changes in a physiological condition of the wearer.

In some embodiments, a system includes a wearable device and a charger case. The wearable device can be configured to be positioned on an ear of a wearer, the wearable device including a first body portion, a second body portion, and a connection member mechanically coupled to each of the first body portion and the second body portion, at least one of the first body portion or the second body portion having a processor, a memory operably coupled to the processor, and a sensor set including at least one of a pulse oximeter, a photoplethysmogram (PPG) sensor, an accelerometer, a temperature sensor, a proximity sensor, an ambient light sensor, or a gyroscopic sensor, the memory storing instructions to cause the processor to calculate a blood oxygen saturation level, and at least two of a pulse rate, a blood pressure level, a blood flow rate, a cardiac output/rhythm, a blood sugar level, a blood carbon monoxide level, or a blood nitrous oxide level. The charger case can be configured to contain and supply power to the wearable device, the charger case being at least one of waterproof or wirelessly chargeable.

In some implementations, the memory stores instructions to cause the processor to calculate at least three of a pulse rate, a blood pressure level, a blood flow rate, a cardiac output/rhythm, a blood sugar level, a blood carbon monoxide level, or a blood nitrous oxide level. For example, the memory can store instructions to cause the processor to calculate three, four, five, six, or all seven of the foregoing bioparameters.

In some implementations, the memory stores instructions to calculate electrocardiogram (ECG) data.

In some implementations, the charger case comprises a pair of electrical contacts configured to exchange at least one of power or data with the wearable device when the wearable device is positioned within the charger case.

In some implementations, the charger case is configured to perform wireless charging of the wearable device when the wearable device is contained within the charger case.

In some implementations, the charger case is waterproof and has an ingress protection rating of at least IP67.

In some implementations, the charger case also includes a battery configured to supply power to the wearable device and a battery monitoring circuit operably coupled to the battery.

In some implementations, the charger case also includes a lid portion and a base portion coupled by a hinge, the lid portion configured to enclose the wearable device when the charger case is in a closed configuration.

In some implementations, the charger case also includes at least one LED indicator configured to indicate a charging status of the wearable device.

In some implementations, the wearable device also includes a first light-emitting diode, a second light-emitting diode, and at least one optical sensor. The first light-emitting diode can be configured to emit light having a first wavelength, the second light-emitting diode can be configured to emit light having a second wavelength different from the first wavelength, and the at least one optical sensor can be configured to detect a reflected portion of the first light and a reflected portion of the second light from a surface of the wearer's ear.

In some implementations, the wearable device also includes a first light-emitting diode, a second light-emitting diode, and a sensor set. The first light-emitting diode can be configured to emit light having a first wavelength, the second light-emitting diode can be configured to emit light having a second wavelength different from the first wavelength, and the sensor set can be an ambient light sensor configured to adjust at least one of an intensity of light emitted by the first light-emitting diode or an intensity of light emitted by the second light-emitting diode, based on detected ambient light conditions.

In some implementations, the charger case also includes a universal serial bus (USB) port configured to enable wired charging of the charger case.

In some implementations, the wearable device also includes an alert mechanism operably coupled to the processor, the memory storing instructions to cause the processor to initiate an emergency plan in response to detecting a user interaction with the alert mechanism.

In some implementations, the wearable device also includes at least one microphone and at least one speaker operably coupled to the processor, the memory storing instructions to cause the processor to activate the microphone and the speaker in response to initiating an emergency plan.

In some embodiments, a system includes a wearable device, a charger case, and a plurality of flexible earpads. The wearable device can be configured to be positioned on an ear of a wearer, the wearable device including a first body portion, a second body portion, and a connection member mechanically coupled to each of the first body portion and the second body portion, at least one of the first body portion or the second body portion having a processor, a memory operably coupled to the processor, a first light-emitting diode, a second light-emitting diode, and at least one optical sensor configured to detect, during operation of the wearable device, a reflected portion of first light emitted from the first light-emitting diode and a reflected portion of second light emitted from the second light-emitting diode, each of the reflected portion of the first light and the reflected portion of the second light being reflected from an associated surface of the ear of the wearer, the memory storing instructions to cause the processor to calculate a blood oxygen saturation level and at least two of a pulse rate, a blood pressure level, a blood flow rate, a cardiac output/rhythm, a blood sugar level, a blood carbon monoxide level, or a blood nitrous oxide level based on at least one of the reflected portion of the first light or the reflected portion of the second light. The charger case can be configured to contain the wearable device and to supply power to the wearable device, the charger case being at least one of waterproof or wireless. Each flexible earpad from the plurality of flexible earpads configured to be mechanically coupled to one of the first body portion or the second body portion.

In some implementations, the memory stores instructions to cause the processor to calculate at least three of a pulse rate, a blood pressure level, a blood flow rate, a cardiac output/rhythm, a blood sugar level, a blood carbon monoxide level, or a blood nitrous oxide level.

In some implementations, the memory also stores instructions to calculate or generate electrocardiogram (ECG) data and, optionally, to generate an ECG.

In some implementations, the wearable device is in wireless communication with a mobile compute device running a software application during operation of the wearable device.

In some implementations, the wearable device also includes at least one microphone and at least one speaker operably coupled to the processor, the memory storing instructions to cause the processor to activate the microphone and the speaker in response to initiating an emergency plan.

In some implementations, the charger case also includes a pair of electrical contacts configured to exchange at least one of power or data with the wearable device when the wearable device is positioned within the charger case.

In some implementations, the charger case is configured to support wireless charging of the wearable device when the wearable device is contained within the charger case.

In some implementations, the charger case is waterproof and has an ingress protection rating of at least IP67.

In some implementations, the charger case also includes a battery configured to supply power to the wearable device and a battery monitoring circuit operably coupled to the battery.

In some implementations, each flexible earpad from the plurality of flexible earpads is configured to be interchangeable with another flexible earpad having different dimensions to customize a fit of the wearable device to the ear of the wearer.

In some implementations, each flexible earpad comprises a silicone material.

In some implementations, the wearable device also includes a first light-emitting diode and a second light-emitting diode. The first light-emitting diode can be configured to emit light having a first wavelength, and the second light-emitting diode can be configured to emit light having a second wavelength different from the first wavelength. The sensor set can include an ambient light sensor configured to adjust an intensity of light emitted by the first light-emitting diode and the second light-emitting diode based on detected ambient light conditions. The wearable device can also include an alert mechanism operably coupled to the processor, the memory storing instructions to cause the processor to initiate an emergency plan in response to detecting a user interaction with the alert mechanism. Alternatively or in addition, the emergency plan can include at least one of initiating a telephone call to emergency services, sending a short message service (SMS) message to a predefined contact, or transmitting an alert signal to a mobile software application.

In some implementations, the memory stores instructions to cause the processor to generate a sleep report based on data collected from at least two of a photoplethysmogram sensor, an accelerometer, or a gyroscopic sensor.

### Wearable Monitor Applications—COVID-19

The novel coronavirus (Coronavirus disease 2019 (COVID-19) is an infectious disease, caused by severe acute respiratory syndrome coronavirus 2 (SARS-CoV-2), that disrupts functions of many organ systems and has resulted in hundreds of thousands of deaths to date. COVID-19 impacts the respiratory system, with effects ranging from mild upper respiratory symptoms to pneumonia and acute respiratory distress syndrome. One potential COVID-19-induced symptom is silent hypoxia, in which a patient experiences below-average (or “low”) tissue and blood oxygen saturation, yet does not exhibit breathlessness. Although the mechanisms through which silent hypoxia emerges are unclear, the phenomenon warrants significant clinical and public health attention. Silent hypoxia presents at least two problems, with regard to public health: 1) Infected individuals may progress to more severe disease states yet may be unaware that they are COVID-positive, and 2) Infected, silently hypoxic patients may serve as vectors to infect those around them (“asymptomatic spread”). For hypoxic patients, continuous blood oxygen saturation monitoring is important. As such, there is a desire for smart, portable, and wearable devices that provide real-time SpO2 monitoring and alarm users when their SpO2 levels drop below acceptable levels.

One or more embodiments of the wearable monitor set forth herein can perform robust blood oxygen saturation level monitoring, for a variety of applications, including: the identification of silently hypoxic patients, helping alert healthcare providers to patients at risk of serious disease progression, augmenting existing COVID-19 diagnostic platforms such as nasopharyngeal swabs, preventing the spread of COVID-19, assisting healthcare providers in the coordination of care, and monitoring one or more of a variety of health conditions detailed below. Normal blood oxygen saturation levels are approximately 95 percent. When a patient exhibits blood oxygen saturation below this threshold, the patient's organ systems, particularly the brain, may receive inadequate oxygen supply, which could lead to confusion or lethargy. If the oxygen level drops below 80 percent, there is risk of serious damage to vital organs, potentially leading to death.

COVID-19 has been reported to induce silent hypoxia in patients. According to Dr. Richard Levitan, an emergency doctor at New York City's Bellevue Hospital, those suffering from COVID-induced silent hypoxia do not experience difficulty breathing until the day they arrive at the hospital. It follows that a subset of COVID-19 patients is entirely asymptomatic despite having COVID-induced hypoxia for a period of time, suggesting that the silently hypoxic patients could spread the infection to others and also progress to more serious COVID-19 stages.

A continuous blood oxygen saturation monitor is desirable, to protect vulnerable members of the population and to facilitate the detection of low blood oxygen saturation as early as possible. High-sensitivity monitoring can help to ensure that patients receive medical assistance before their condition deteriorates, thereby improving patient outcomes and helping the healthcare system manage the current burden of the pandemic.

### COVID-19 Symptoms and Risk Factors

Fever, designated by the Centers for Disease Control and Prevention as body temperature above 100.4 degrees Celsius, is the symptom most commonly associated with COVID-19 among the public. However, clinical observational analysis at Northwell Health, the largest provider system in New York, indicates that only 30.7% of patients diagnosed with COVID-19 were febrile upon clinical presentation. The weak correlation between fever and infection suggests that the presentation of COVID-19 is enormously variable, and that providers should screen patients for multiple parameters when assessing the presence or severity of infection.

Although the relationship between febrility and infection is unclear, clinical studies demarcate specific risk factors that can make patient populations more vulnerable to serious infection. According to observations of COVID-19 positive patients at Northwell Health, 56.6% had a history of hypertension, 41.7% had a history of obesity, and 33.8% were diabetic. The strong correlations between chronic pre-existing conditions and COVID-19 hospitalization underscore the desirability of robust monitoring, particularly of high-risk populations.

Clinical observations also suggest the desirability of prognostic markers for COVID-19, which could help ensure that patients are treated earlier and more aggressively, and help in preventing progression to mechanical ventilation, which is associated with significantly higher mortality. In connection with planned reopenings of workplaces and schools, some entities are planning to use thermal scanners to flag febrile individuals. Afebrile individuals, however, may act as vectors for the spread of infection despite not exhibiting this benchmark symptom of COVID-19. More expansive symptom tracking could help to contain the spread of COVID-19.

### Risk of COVID-19 Among Elderly and Significance of Hypertension

Individuals older than 65 are overrepresented in the composition of COVID-19 infection, hospitalization, ICU admission, and death. The greater risk COVID-19 that poses to the elderly has greatly contributed to social distancing and the implementation of other public health measures intended to slow the spread of the disease. The strong correlation between hypertension and COVID-19 is believed to play a central role in the overrepresentation of the elderly among infected patients. 63.1% of adults above the age of 60 are hypertensive, and many in this patient population use Angiotensin Converting Enzyme (ACE) inhibitors to lower their blood pressure. Sustained use of ACE inhibitors may serve to increase expression of the ACE-2 receptor in the cardiovascular, renal, gastrointestinal, and pulmonary organ systems. Because SARS-CoV-2 is believed to enter cells through the ACE-2 receptor, the upregulation of ACE-2 in hypertensive patients, who are disproportionately elderly, could serve to increase their disease burden.

### COVID-19 and Hospital Transmission for High-Risk Patients

Dr. Shu-Yuan Xiao, a gastroenterologist affiliated with the University of Chicago School of Medicine, studied two patients in Zhongnan hospital in Wuhan, China. The patients, undergoing lung lobectomies for adenocarcinoma, were found to have COVID-19 during the surgery. Pathology reports revealed that both patients had pulmonary edema and inflamed alveoli, both characteristic of pneumonia. At the time of the surgery, neither patient exhibited symptoms of pneumonia, suggesting that patients remained in an early phase of the disease progression. The unexpected cases of pneumonia in a patient population reporting for a surgical procedure for a non-COVID condition illustrates the risk of asymptomatic patients serving as vectors for healthcare providers and other patients in the hospital. To counteract the spread of COVID-19 within healthcare settings, aggressive monitoring of symptoms is desirable, to isolate patients suspected to be positive.

### Silent Hypoxia: A Clinical Conundrum

Hypoxemia refers to below-average blood oxygenation, while hypoxia is the clinical state of diminished tissue oxygen tension. Clinically, normal arterial blood oxygen saturation is designated as between 94% and 100%. Reduced blood and tissue oxygenation can disrupt cellular metabolism, growth, and development. Healthcare professionals have traditionally understood hypoxia to be coincident with symptoms of respiratory distress, such as shortness of breath. The clinical presentation of COVID-19, however, partly contradicts this understanding. Dr. Richard Levitan, a volunteer emergency medical provider at Bellevue Hospital in New York, reports cases in which patients, despite having undergone imaging that confirmed viral pneumonia and sub-average blood oxygen saturation, did not report respiratory discomfort for several days before presenting at a hospital. Such cases document a phenomenon referred to as “silent hypoxia.” referenced above Classification levels for hypoxemia are presented in the following table:

CLASSIFICATION OF HYPOXEMIA

| | PaO₂ | SaO₂ |
|---|---|---|
| Classification | (mmhg) | (%) |
| Normal | 80-100 | >95 |
| Mild Hypoxemia | 60-70 | 90-94 |
| Moderate Hypoxemia | 40-59 | 75-89 |
| Severe Hypoxemia | <40 | <75 |

COVID-19 binds to receptors on alveolar cells, which produce surfactant. Surfactant breaks up the surface tension of water within the alveoli, thereby preventing the alveolar space from collapsing following exhalation. Infection contributes to reduced surfactant production and collapsed air spaces. White blood cells, or leukocytes, mount an inflammatory response within the alveoli. Leukocytes also release cytokines which promote fluid leakage from the pulmonary microvasculature into the space around the alveoli. The buildup of fluid also contributes to the alveolar collapse. Alveolar collapse reduces the interface for oxygen to diffuse into the bloodstream, contributing to hypoxemia. At this stage in disease progression, the lung's compliance may remain unchanged. Consequently, patients may still be able to exhale normal amounts of carbon dioxide, which prevents the onset of breathlessness—making them “silently hypoxic.” In certain cases, the buildup of fluid and inflammation can progress to a point in which total lung volume decreases, which impedes the clearance of carbon dioxide and results in breathlessness. This transition can be rapid and necessitate ventilatory assistance, which can burden the healthcare system in the aggregate.

### COVID-19 and ARDS

Acute respiratory distress syndrome (ARDS) is characterized by the acute development of pulmonary edema, hypoxia, and a subsequent reliance on mechanical ventilation. ARDS is a prominent cause of respiratory failure and was evident in 10% of patients in ICUs prior to the emergence of COVID-19. Unlike cases of silent hypoxia, ARDS is associated with diminished exhalation of carbon dioxide, which contributes to increased shortness of breath. Severe injury to the alveolar cells of the lung contributes to ARDS. Alveolar damage results in an increase in alveolar permeability to fluid—a process that is mediated by inflammatory signals known as cytokines. Disease progression can lead to widespread pulmonary scarring and adverse changes in lung compliance.

ARDS has particular relevance with respect to COVID-19. A retrospective clinical study of 107 patients in Wuhan, China, indicated that 26.2% of all COVID patients had developed ARDS. Among deceased COVID patients, 78.9% had reported ARDS. Another Wuhan clinical study indicates that patients developed ARDS between 8 and 15 days after the onset of illness. The seemingly benign hypoxia in “silent hypoxia” can progress to ARDS, which can be fatal for COVID patients. As such, robust monitoring of blood oxygen saturation can alert patients of hypoxia before the onset of ARDS, potentially improving outcomes and helping providers organize care.

### Clinical Management of ARDS

Severe inflammation and fluid accumulation within the lungs of ARDS patients can result in advanced lung fibrosis potentially culminating in total lung collapse. According to the American Thoracic Society, ARDS is associated with a 30-40% mortality rate. Among patients who recover, lung function can gradually recover in a process that can take between six months to a year. Such recovery is only partial, however, as surviving patients will have below-average lung volume and remaining lung fibrosis. According to Dr. Gregory Cosgrove, Chief Medical Officer of the Pulmonary Fibrosis Foundation, patients who survive ARDS can have reduced quality of life which can contribute to anxiety, depression, and/or PTSD. Thus, it is desirable for clinical workflows to include symptom monitoring to rapidly identify patients whose mild COVID-19 symptoms may progress to more severe states such as ARDS.

### Clotting and Silent Hypoxia

With increased awareness regarding silent hypoxia in COVID-19 patients, theories have emerged regarding the origin of the phenomenon which seems to contradict medical convention. Dr. Elnara Marcia Negri, a pulmonologist in Sao Paulo, Brazil, emphasizes the role of clotting in inducing a silent hypoxic state in COVID-19 patients. According to Negri, an inflammatory reaction within the pulmonary vasculature may result in subtle increases in blood clot formation in patients with COVID-19. Increased clot formation would interfere with the diffusion of oxygen from the alveoli into the bloodstream, resulting in hypoxia. Negri administered heparin, a common anticoagulant, to patients with hypoxia, regardless of whether they experienced symptoms. According to Negri, 24 of 27 patients have recovered, lending support to her theory. Negri advises patients to routinely monitor their blood oxygen saturation levels, visiting the hospital in the event that their blood oxygen saturation levels dip below 93%.

### Hypoxic Strokes in COVID-19

Large-vessel stroke is a condition in which blood flow in one of the major arteries perfusing the brain becomes interrupted. Interrupted blood flow reduces the availability of oxygen in the brain. Physicians affiliated with the Mount Sinai Health System in New York reported five cases of stroke in COVID-19 positive patients below the age of 50 years old. Furthermore, a retrospective study of data from Wuhan indicates that the incidence of stroke among COVID-19 patients was approximately 5%.

Additionally, the average age of COVID-19 patients afflicted with strokes in Wuhan was 55 years, suggesting that COVID-19-related strokes pose a distinctive risk to a slightly younger patient population. Physicians attribute the incidence of strokes in COVID-19 patients to the role the infection plays in inducing dysfunction in endothelial cells, which line the interior of blood vessels. The association of COVID-19 with other serious pathologies, such as large-vessel strokes, underscores the desirability of robust monitoring of blood oxygen saturation.

### Shortcomings in Known COVID-19 Diagnostic Technology

Cleveland Clinic researchers have investigated the efficacy of existing diagnostic technology used to identify patients who are positive for COVID-19. These diagnostic tests include Abbott's ID NOW machine, which has been said to produce results in under 15 minutes. According to the study, the ID NOW had a false-negative rate of 14.8% and a true positive rate of 85.2%. The study's results suggest that approximately 15% of positive patients would falsely be labeled as being uninfected with the ID NOW test. The study also investigated the DiaSorin Simplexa test, which had a true-positive rate of 89.3%. According to Dr. Gary Procop, head of COVID-19 testing at the Cleveland Clinic, diagnostic tests should have true-positive rates of at least 95% to assure the public of the test's efficacy. Although diagnostic platforms made by Roche and Cepheid had accuracy rates above 95%, the subthreshold accuracy of the DiaSorin Simplexa and ID NOW platforms suggest that there are lingering inadequacies within areas of diagnostics for COVID-19. The challenges within COVID-19 testing render the monitoring of other clinical variables, like blood oxygen saturation, even more important. It is possible that robust oxygen monitoring could be used to supplement traditional nasopharyngeal swab tests to yield an even higher accuracy of COVID-19 diagnosis, quickly identifying patients who need medical assistance.

### Known Monitoring Solutions to Combat Silent Hypoxia—Pulse Oximetry

Given the possibility that a patient may experience severe hypoxia prior to the onset of breathlessness, some medical providers have called for widespread pulse oximetry. Pulse oximetry can provide patients with a non-invasive way to monitor their blood oxygen saturation levels, alarming them of hypoxia even if they report no other symptoms. A pulse oximeter includes a light emitting sensor that can be clipped to a patient's finger. Patients using at-home pulse oximeters may consult their medical providers, facilitating proper interpretation of blood oxygen saturation levels. Dr. Levitan, an emergency medical physician at Bellevue hospital who noted cases of silent hypoxia, has called for all COVID-19 positive patients to routinely check their blood oxygen saturation levels within the two weeks following diagnosis. Oxygenation monitoring for patients that have not been diagnosed, yet have symptoms of cough, fatigue, and/or fever, may also be prudent

Known pulse oximeters, such as finger clips, can be useful for patient assessment, but are often difficult to transport and/or use. Some pulse oximeters, such as the Nonin Onyx, are bulky and can fall off during patient transport or as a result of routine daily movements such as getting up, sitting down, and wiggling one's fingers. In addition, some patients may have a weak pulse or tissue damage, which can skew measurement results. Other known wearable devices provide measurements only at the wrist, and such devices typically do not provide oxygen monitoring or associated warnings. Rather, such devices measure pulse and fitness-related measurements such as step count and sleep time.

In the case of COVID-19, the pulse oximeter finger cuff typically does not provide and/or is not used for continuous monitoring. In cases of silent hypoxia, when patients do not feel any respiratory distress, it may not occur to patients to check their blood oxygen saturation levels, and be unaware of their hypoxia for extended periods of time.

As discussed above, blood oxygen saturation is a valuable biomarker of COVID-19, both for symptomatic and asymptomatic individuals. Embodiments of the wearable monitor set forth herein facilitate the continuous monitoring of blood oxygen saturation levels, via measurements taken on a wearer's ear (e.g., the helix, scapha, pinna, etc.), with relevant measurement data displayed via a mobile software application. When the wearer's SpO₂ levels drop below acceptable levels, an alarm is generated and communicated to the wearer (e.g., via one or more of: an audio indication, a light indication, a GUI display, etc.). In addition, the wearer can trigger a call to emergency services by clicking a button on the wearable monitor.

All combinations of the foregoing concepts and additional concepts discussed herewithin (provided such concepts are not mutually inconsistent) are contemplated as being part of the subject matter disclosed herein. The terminology explicitly employed herein that also may appear in any disclosure incorporated by reference should be accorded a meaning most consistent with the particular concepts disclosed herein.

The drawings are primarily for illustrative purposes, and are not intended to limit the scope of the subject matter described herein. The drawings are not necessarily to scale; in some instances, various aspects of the subject matter disclosed herein may be shown exaggerated or enlarged in the drawings to facilitate an understanding of different features. In the drawings, like reference characters generally refer to like features (e.g., functionally similar and/or structurally similar elements).

The entirety of this application (including the Cover Page, Title, Headings, Background, Summary, Brief Description of the Drawings, Detailed Description, Embodiments, Abstract, Figures, Appendices, and otherwise) shows, by way of illustration, various embodiments in which the embodiments may be practiced. The advantages and features of the application are of a representative sample of embodiments only, and are not exhaustive and/or exclusive. Rather, they are presented to assist in understanding and teach the embodiments, and are not representative of all embodiments. As such, certain aspects of the disclosure have not been discussed herein. That alternate embodiments may not have been presented for a specific portion of the innovations or that further undescribed alternate embodiments may be available for a portion is not to be considered to exclude such alternate embodiments from the scope of the disclosure. It will be appreciated that many of those undescribed embodiments incorporate the same principles of the innovations and others are equivalent. Thus, it is to be understood that other embodiments may be utilized and functional, logical, operational, organizational, structural and/or topological modifications may be made without departing from the scope and/or spirit of the disclosure. As such, all examples and/or embodiments are deemed to be non-limiting throughout this disclosure.

Also, no inference should be drawn regarding those embodiments discussed herein relative to those not discussed herein other than it is as such for purposes of reducing space and repetition. For instance, it is to be understood that the logical and/or topological structure of any combination of any program components (a component collection), other components and/or any present feature sets as described in the figures and/or throughout are not limited to a fixed operating order and/or arrangement, but rather, any disclosed order is exemplary and all equivalents, regardless of order, are contemplated by the disclosure.

The term “automatically” is used herein to modify actions that occur without direct input or prompting by an external source such as a user. Automatically occurring actions can occur periodically, sporadically, in response to a detected event (e.g., a user logging in), or according to a predetermined schedule.

As used herein, the term “substantially” has a meaning similar to “mostly” or “to a great extent.” For example, the phrase “a substantially uniform thickness” refers to a thickness value plus or minus a range of 10%.

The term “determining” encompasses a wide variety of actions and, therefore, “determining” can include calculating, computing, processing, deriving, investigating, looking up (e.g., looking up in a table, a database or another data structure), ascertaining and the like. Also, “determining” can include receiving (e.g., receiving information), accessing (e.g., accessing data in a memory) and the like. Also, “determining” can include resolving, selecting, choosing, establishing and the like.

The phrase “based on” does not mean “based only on,” unless expressly specified otherwise. In other words, the phrase “based on” describes both “based only on” and “based at least on.”

The term “processor” should be interpreted broadly to encompass a general purpose processor, a central processing unit (CPU), a microprocessor, a digital signal processor (DSP), a controller, a microcontroller, a state machine and so forth. Under some circumstances, a “processor” may refer to an application specific integrated circuit (ASIC), a programmable logic device (PLD), a field programmable gate array (FPGA), etc. The term “processor” may refer to a combination of processing devices, e.g., a combination of a DSP and a microprocessor, a plurality of microprocessors, one or more microprocessors in conjunction with a DSP core or any other such configuration.

The term “memory” should be interpreted broadly to encompass any electronic component capable of storing electronic information. The term memory may refer to various types of processor-readable media such as random access memory (RAM), read-only memory (ROM), non-volatile random access memory (NVRAM), programmable read-only memory (PROM), erasable programmable read only memory (EPROM), electrically erasable PROM (EEPROM), flash memory, magnetic or optical data storage, registers, etc. Memory is said to be in electronic communication with a processor if the processor can read information from and/or write information to the memory. Memory that is integral to a processor is in electronic communication with the processor.

The terms “instructions” and “code” should be interpreted broadly to include any type of computer-readable statement(s). For example, the terms “instructions” and “code” may refer to one or more programs, routines, sub-routines, functions, procedures, etc. “Instructions” and “code” may comprise a single computer-readable statement or many computer-readable statements.

Some embodiments described herein relate to a computer storage product with a non-transitory computer-readable medium (also can be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also can be referred to as code) may be those designed and constructed for the specific purpose or purposes. Examples of non-transitory computer-readable media include, but are not limited to, magnetic storage media such as hard disks, floppy disks, and magnetic tape; optical storage media such as Compact Disc/Digital Video Discs (CD/DVDs), Compact Disc-Read Only Memories (CD-ROMs), and holographic devices; magneto-optical storage media such as optical disks; carrier wave signal processing modules; and hardware devices that are specially configured to store and execute program code, such as Application-Specific Integrated Circuits (ASICs), Programmable Logic Devices (PLDs), Read-Only Memory (ROM) and Random-Access Memory (RAM) devices. Other embodiments described herein relate to a computer program product, which can include, for example, the instructions and/or computer code discussed herein.

Some embodiments and/or methods described herein can be performed by software (executed on hardware), hardware, or a combination thereof. Hardware modules may include, for example, a general-purpose processor, a field programmable gate array (FPGA), and/or an application specific integrated circuit (ASIC). Software modules (executed on hardware) can be expressed in a variety of software languages (e.g., computer code), including C, C++, Java™, Ruby, Visual Basic™, and/or other object-oriented, procedural, or other programming language and development tools. Examples of computer code include, but are not limited to, micro-code or micro-instructions, machine instructions, such as produced by a compiler, code used to produce a web service, and files including higher-level instructions that are executed by a computer using an interpreter. For example, embodiments may be implemented using imperative programming languages (e.g., C, Fortran, etc.), functional programming languages (Haskell, Erlang, etc.), logical programming languages (e.g., Prolog), object-oriented programming languages (e.g., Java, C++, etc.) or other suitable programming languages and/or development tools. Additional examples of computer code include, but are not limited to, control signals, encrypted code, and compressed code.

Various concepts may be embodied as one or more methods, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments. Put differently, it is to be understood that such features may not necessarily be limited to a particular order of execution, but rather, any number of threads, processes, services, servers, and/or the like that may execute serially, asynchronously, concurrently, in parallel, simultaneously, synchronously, and/or the like in a manner consistent with the disclosure. As such, some of these features may be mutually contradictory, in that they cannot be simultaneously present in a single embodiment. Similarly, some features are applicable to one aspect of the innovations, and inapplicable to others.

In addition, the disclosure may include other innovations not presently described. Applicant reserves all rights in such innovations, including the right to embodiment such innovations, file additional applications, continuations, continuations-in-part, divisionals, and/or the like thereof. As such, it should be understood that advantages, embodiments, examples, functional, features, logical, operational, organizational, structural, topological, and/or other aspects of the disclosure are not to be considered limitations on the disclosure as defined by the embodiments or limitations on equivalents to the embodiments. Depending on the particular desires and/or characteristics of an individual and/or enterprise user, database configuration and/or relational model, data type, data transmission and/or network framework, syntax structure, and/or the like, various embodiments of the technology disclosed herein may be implemented in a manner that enables a great deal of flexibility and customization as described herein.

All definitions, as defined and used herein, should be understood to control over dictionary definitions, definitions in documents incorporated by reference, and/or ordinary meanings of the defined terms.

As used herein, in particular embodiments, the terms “about” or “approximately” when preceding a numerical value indicates the value plus or minus a range of 10%. Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. That the upper and lower limits of these smaller ranges can independently be included in the smaller ranges is also encompassed within the disclosure, subject to any specifically excluded limit in the stated range. Where the stated range includes one or both of the limits, ranges excluding either or both of those included limits are also included in the disclosure.

The indefinite articles “a” and “an,” as used herein in the specification and in the embodiments, unless clearly indicated to the contrary, should be understood to mean “at least one.”

The phrase “and/or,” as used herein in the specification and in the embodiments, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the embodiments, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the embodiments, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the embodiments, shall have its ordinary meaning as used in the field of patent law.

As used herein in the specification and in the embodiments, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the embodiments, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. An apparatus, comprising:
a wearable device configured to be positioned on an ear of a wearer, the wearable device including a first body portion, a second body portion, and a connection member mechanically coupled to each of the first body portion and the second body portion, at least one of the first body portion or the second body portion having a processor, a memory operably coupled to the processor, and a sensor set including at least one of a pulse oximeter, a photoplethysmography (PPG) sensor, an accelerometer, a temperature sensor, a proximity sensor, an ambient light sensor, or a gyroscopic sensor, the memory storing instructions to cause the processor to calculate a blood oxygen saturation level of the wearer and at least two of:
calculate a pulse rate of the wearer;
calculate a blood pressure level of the wearer;
calculate a blood flow rate of the wearer;
identify a cardiac output/rhythm of the wearer;
calculate a blood sugar level of the wearer;
calculate a blood carbon monoxide level of the wearer;
calculate a blood nitrous oxide level of the wearer;
generate an electrocardiogram associated with the wearer;
estimate a risk of one of dementia or Alzheimer's disease of the wearer by analyzing at least one of blood pressure variability (BPV), pulse wave velocity (PWV), and pulse transit time (PTT); or
detect peripheral artery disease (PAD) of the wearer based on a pulse wave form detected via the wearable device.

2. The apparatus of claim 1, wherein the wearable device further comprises:
a first light-emitting diode configured to emit light having a first wavelength,
a second light-emitting diode configured to emit light having a second wavelength different from the first wavelength, and
at least one optical sensor configured to detect a reflected portion of the first light and a reflected portion of the second light from a surface of the ear of the wearer.

3. The apparatus of claim 2, wherein the first wavelength is in a red light spectrum and the second wavelength is in an infrared spectrum, and the processor is configured to calculate the blood oxygen saturation level of the wearer based on a Beer-Lambert law and using an amount of light absorbed by the surface of the ear of the wearer.

4. The apparatus of claim 1, wherein the sensor set includes the PPG sensor, and the PPG sensor is configured to detect the pulse wave form based on measured time intervals between heartbeats.

5. The apparatus of claim 4, wherein the PPG sensor comprises:
one or more light-emitting diodes (LEDs) configured to emit light at or toward a tissue site of the ear of the wearer, and
a photodiode configured to measure an intensity of non-absorbed light reflected from the tissue site.

6. The apparatus of claim 5, wherein the one or more LEDs includes at least one of a red LED, a green LED, or a yellow LED.

7. The apparatus of claim 1, wherein the sensor set also includes at least one of an accelerometer or a gyroscopic sensor and is configured to detect at least one of a body position, a head position, or a fall event of the wearer.

8. The apparatus of claim 1, wherein:
the wearable device further comprises:
a first light-emitting diode configured to emit light having a first wavelength, and
a second light-emitting diode configured to emit light having a second wavelength different from the first wavelength; and
the sensor set includes an ambient light sensor,
the apparatus configured to adjust an intensity of light emitted by the first light-emitting diode and the second light-emitting diode based on detected ambient light conditions.

9. The apparatus of claim 1, wherein the sensor set also includes a proximity sensor configured to detect at least one of a position or an orientation of the wearable device relative to the ear of the wearer.

10. The apparatus of claim 1, wherein the wearable device further comprises:
a vibration motor operably coupled to the processor, the memory storing instructions to cause the processor to operate the vibration motor in response to detecting an alarm condition.

11. The apparatus of claim 1, wherein the wearable device further comprises:
a wireless transceiver operably coupled to the processor, and configured to communicate with a mobile software application.

12. The apparatus of claim 11, wherein the memory stores instructions to cause the processor to transmit measurement data to the mobile software application via the wireless transceiver using at least one of Bluetooth®, LTE, or Wi-Fi protocol.

13. The apparatus of claim 1, wherein the memory stores instructions to cause the processor to generate an alert when the calculated blood oxygen saturation level is below a predefined threshold, the alert comprising at least one of an audible sound, a vibration, or a visual indication.

14. The apparatus of claim 1, wherein the wearable device further comprises:
an alert mechanism operably coupled to the processor, the memory storing instructions to cause the processor to initiate an emergency plan in response to detecting a user interaction with the alert mechanism.

15. The apparatus of claim 14, wherein the emergency plan comprises at least one of: initiating a telephone call to emergency services, sending a short message service (SMS) message to a predefined contact, or transmitting an alert signal to a mobile software application.

16. The apparatus of claim 1, wherein the memory further stores instructions to cause the processor to estimate a risk of dementia or Alzheimer's disease by analyzing at least one of blood pressure variability, pulse wave velocity, or pulse transit time detected via the sensor set.

17. The apparatus of claim 1, wherein the wearable device further comprises:
a temperature sensor configured to measure a skin temperature of the wearer, the wearable device configured to recalculate the blood oxygen saturation based on the measured temperature.

18. The apparatus of claim 1, wherein the wearable device further comprises a battery voltage sensor, and the wearable device is configured to regulate a power consumption by adjusting an operation of the sensor set based on an activity level of the wearer and a battery voltage detected by the battery voltage sensor.

19. The apparatus of claim 1, wherein the memory further stores instructions to cause the processor to generate a sleep report based on data collected from at least two sensors from the sensor set.

20. The apparatus of claim 1, wherein the wearable device further comprises:
at least one microphone and at least one speaker operably coupled to the processor, the memory storing instructions to cause the processor to activate the microphone and the speaker in response to initiating an emergency plan.

21. The apparatus of claim 1, wherein the wearable device is configured to continuously monitor a blood oxygen saturation level of the wearer and to dynamically adjust a sampling rate of the sensor set based on detected changes in a physiological condition of the wearer.
